(19) United States
(12) Patent Application Publication
(10) Pub. No.: US 2022/0362245 A1
(43) Pub. Date: Nov. 17, 2022

(54) COMPOUNDS FOR INHIBITING FGFR4

(71) Applicant: Terns Pharmaceuticals, Inc., Foster City, CA (US)

(72) Inventors: Yingzi XU, Palo Alto, CA (US); F. Anthony ROMERO, Redwood City, CA (US)

(73) Assignees: Terns Pharmaceuticals, Inc., Foster City, CA (US); Terns Pharmaceuticals, Inc., Foster City, CA (US)

(21) Appl. No.: 17/621,203

(22) PCT Filed: Jun. 18, 2020
(86) PCT No.: PCT/US2020/038541

§ 371 Date: Dec. 20, 2021

Related U.S. Application Data
(60) Provisional application No. 62/864,883, filed on Jun. 21, 2019.

(51) Int. Cl. A61K 31/506; C07D 471/04; A61K 31/47; A61K 39/395; A61K 31/44

(52) U.S. Cl. CPC A61K 31/506; C07D 471/04; A61K 31/47; A61K 39/3955; A61K 31/44

(57) ABSTRACT

Provided herein are compounds which inhibit FGFR4, compositions thereof, methods of their preparation, and methods for treating disorders mediated by FGFR4 such as cancer.

### CROSS REFERENCE TO RELATED APPLICATION

This application claims priority to U.S. Provisional Application No. 62/864,883, filed Jun. 21, 2019, which is incorporated herein by reference in its entirety.

### FIELD

The present disclosure relates generally to compounds and compositions for inhibition of FGFR4, methods of their preparation, and their use in treating cancers.

### BACKGROUND

Fibroblast Growth Factors (FGFs) are a family of polypeptides known to be important for growth, tissue repair, tissue remodeling, wound healing, cellular proliferation, cell migration and differentiation, hematopoiesis, angiogenesis, and tumorigenesis. Many FGFs act through FGF receptors (FGFRs), a group of cell surface receptors in the Receptor Protein Tyrosine Kinase (RPTK) family. Four FGFRs have been identified to date: FGFR1, FGFR2, FGFR3, and FGFR4.

FGFR aberrations have been associated with many cancers. FGFR4 has been reported to play an important role in liver cancer in particular (French, et al., PLoS One, 2012, 7(5): e36713), although FGFR4 and/or the FGFR4 ligand FGF19 have been implicated in other cancer types including breast, uterine, glioblastoma, prostate, rhabdomyosarcoma, gastric, ovarian, lung, and colorectal cancer (Jaakkola, et al., Int. J. Cancer, 1993; 54(3):378-382; Masica, et al., Cancer Res 2011; 71(13):4550-4561; Feng, et al., Cancer Res, 2013, 73(8):2551-2562; Crose, et al., Clin Cancer Res, 2012, 18(14):3780-3790; Ye, et al., Ann Surg Oncol, 2010, 17(12):3354-61; Fawder, et al., PNAS, 2013, 110(30):12426-12431; Turkington et al, Cell Death & Disease, 2014, 5: e1046). Thus, therapeutic agents that function as inhibitors of FGFR4 have the potential to treat certain cancers, including liver cancer such as hepatocellular carcinoma (HCC).

Pan-FGFR inhibitors have a number of dose-limiting toxicities, including hyperphosphataemia (Chae, et al., Oncotarget, 2017; 8(9):16052-16074). Hyperphosphataemia is caused by the blockage of FGF23 signaling, predominantly through FGFR1 or the combination of FGFR1 and FGFR4 (Gattineni, et al., Am J Physiol Renal Physiol., 2009; 297(2): F282-F291; Gattineni, et al., Am J Physiol Renal Physiol., 2014; 306: F351-F358). As such, selective FGFR4 inhibitors that do not inhibit FGFR1 would have a better safety profile than pan-FGFR inhibitors or FGFR1/FGFR4 dual inhibitors.

Provided herein are compounds and compositions that inhibit FGFR4 and are useful for treating disorders mediated by FGFR4.

### BRIEF SUMMARY

In one aspect, provided is a compound of Formula (I)

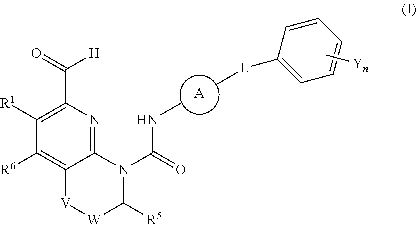

or a pharmaceutically acceptable salt thereof, wherein:

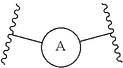

is 5- to 6-membered arylene or heteroarylene, each of which is optionally substituted by 1-4 halogen or C₁-C₆ alkyl groups;

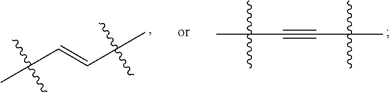

L is —OCH₂—, —CH₂O—, —CH₂CH₂—,
each Y is independently halogen or —O(C₁-C₆ alkyl) optionally substituted by 1-5 groups independently selected from the group consisting of halogen, hydroxyl, —CN, and —NH₂, provided that when

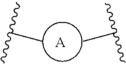

is 5-membered heteroarylene, then at least one Y, when present, is halogen;
n is 0-5;
V is CH₂, O, or CH(OH);
W is CH₂, CH₂CH₂, or a bond;
R¹ is H, halogen, hydroxyl, —CN, —NH₂, C₁-C₆ alkyl, C₁-C₆ haloalkyl, C₁-C₆ alkyl-OH, C₃-C₆ cycloalkyl, —CH₂NR²R³, —CH(CH₃)NR²R³, —O(C₁-C₆ alkyl), —CH₂CO₂H, —C(O)H, or 5- to 6-membered heterocyclyl or heteroaryl,
   wherein the heterocyclyl or heteroaryl contains 1-3 heteroatoms selected from the group consisting of N, O, and S, and each of which heterocyclyl or heteroaryl is optionally substituted by 1-5 groups independently selected from the group consisting of C₁-C₆ alkyl, C₁-C₆ haloalkyl, halogen, hydroxyl, —CN, —NH₂, oxo, and 4- to 6-membered heterocyclyl containing 1-3 heteroatoms selected from the group consisting of N, O, and S;
R² is H, C₁-C₆ alkyl, C₁-C₆ haloalkyl, C₁-C₆ alkyl-OH, or (C₁-C₆ alkyl)₂N—(C₁-C₆ alkylene);
R³ is H, C₁-C₆ alkyl, —C(O)(C₁-C₆ alkyl), C₁-C₆ haloalkyl, C₁-C₆ alkyl-OH, —C(O)CH₂OH, —C(O)CH₂O(C₁-C₆ alkyl), —C(O)CH₂N(C₁-C₆ alkyl)₂, or —S(O)₂(C₁-C₆ alkyl);
or R² and R³ are taken together with the nitrogen atom to which they are attached to form a 5- to 6-membered heterocyclyl optionally containing one additional heteroatom or heteroatom-containing moiety selected from the group consisting of N, N-oxide, O, and S, wherein the heterocyclyl is optionally substituted by 1-5 R⁴ groups;
each R⁴ is independently:
   halogen, —CN, C₁-C₆ alkyl, C₁-C₆ haloalkyl, C₁-C₆ alkyl-OH, —N(C₁-C₆ alkyl)₂, —C(O)(C₁-C₆ alkyl), or hydroxyl;
   taken together with another R⁴ group and the carbon atom or atoms to which they are attached to form a spiro or fused 4- to 6-membered heterocyclyl containing 1-3 heteroatoms selected from the group consisting of N, O, and S; or
   taken together with another R⁴ group attached to the same ring atom to form an oxo group;
R⁵ is H, C₁-C₆ alkyl, or C₁-C₆ cycloalkyl; and
R⁶ is H, halogen, C₁-C₆ alkyl, C₁-C₆ haloalkyl, or C₁-C₆ alkyl-OH.

In some embodiments,

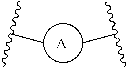

is phenylene or 5- to 6-membered heteroarylene containing 1-3 nitrogen atoms, each of which phenylene or heteroarylene is optionally substituted by 1-2 halogen or C₁-C₃ alkyl groups. In some embodiments,

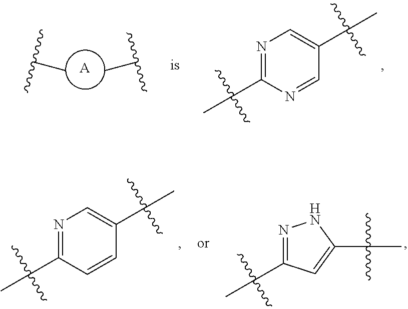

each of which is optionally substituted by 1-2 Cl or —CH₃ groups.

In some embodiments, L is —OCH₂—. In some embodiments, L is —CH₂O—. In some embodiments, L is —CH₂CH₂—. In some embodiments, L is

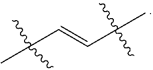 [SMILES: */C=C/*]

In some embodiments, L is

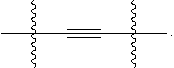 [SMILES: *C#C*]

In some embodiments, each Y, where present, is independently halo or —O(C₁-C₃ alkyl) optionally substituted by 1-3 groups independently selected from the group consisting of halogen, hydroxyl, —CN, and —NH₂. In some embodiments, each Y is independently F, Cl, or —O(C₁-C₂ alkyl) optionally substituted by 1-2 groups independently selected from the group consisting of Cl, hydroxyl, —CN, and —NH₂. In some embodiments, each Y is independently F, Cl or —OCH₃.

In some embodiments, n is 1-5. In some embodiments, n is 4.

In some embodiments, V is CH₂. In some embodiments, V is O or CH(OH).

In some embodiments, W is CH₂. In some embodiments, W is CH₂CH₂ or a bond.

In some embodiments, R¹ is H, halogen, hydroxyl, —CN, —NH₂, C₁-C₃ alkyl, C₁-C₃ haloalkyl, C₁-C₃ alkyl-OH, C₁-C₆ cycloalkyl, —CH₂NR²R³, —CH(CH₃)NR²R³, —O(C₁-C₃ alkyl), —CH₂CO₂H, —C(O)H, or 5- to 6-membered heterocyclyl or heteroaryl, wherein the heterocyclyl or heteroaryl contains 1-3 heteroatoms selected from the group consisting of N, O, and S, and each of which heterocyclyl or heteroaryl is optionally substituted by 1-3 groups independently selected from the group consisting of C₁-C₃ alkyl, C₁-C₃ haloalkyl, halogen, hydroxyl, —CN, —NH₂, oxo, and 4- to 5-membered heterocyclyl containing 1-2 heteroatoms selected from the group consisting of N and O. In some embodiments, R¹ is H, Cl, —CH₃, hydroxyl, —CN, —NH₂, —CF₃, —CH₂OH, cyclohexyl, —CH₂NR²R³, —CH(CH₃)NR²R³, —OCH₃, —CH₂CO₂H, —C(O)H, or 5- to 6-membered heterocyclyl or heteroaryl, wherein the heterocyclyl or heteroaryl contains 1-2 heteroatoms selected from the group consisting of N, O, and S, and each of which heterocyclyl or heteroaryl is optionally substituted by 1-3 groups independently selected from the group consisting of —CH₃, —CF₃, Cl, F, hydroxyl, —CN, —NH₂, oxetanyl, and oxo. In some embodiments, R¹ is H. In some embodiments, R¹ is —CH₂NR²R³.

In some embodiments, R² is H, C₁-C₃ alkyl, C₁-C₃ haloalkyl, C₁-C₃ alkyl-OH, or (C₁-C₃ alkyl)₂N—(C₁-C₃ alkylene); and R³ is H, C₁-C₃ alkyl, C₁-C₃ haloalkyl, C₁-C₃ alkyl-OH, —C(O)(C₁-C₃ alkyl), —C(O)CH₂OH, —C(O)CH₂O(C₁-C₃ alkyl), —C(O)CH₂N(C₁-C₃ alkyl)₂, or —S(O)₂(C₁-C₃ alkyl); or R² and R³ are taken together with the nitrogen atom to which they are attached to form a 5- to 6-membered heterocyclyl optionally containing one additional heteroatom or heteroatom-containing moiety selected from the group consisting of N, N-oxide, O, and S, wherein the heterocyclyl is optionally substituted by 1-5 R⁴ groups. In some embodiments, R² is H, —CH₃, —CF₃, —CH₂OH, or (CH₃)₂N—CH₂—; and R³ is H, —CH₃, —CF₃, —CH₂OH, —C(O)(CH₃), —C(O)CH₂OH, —C(O)CH₂OCH₃, —C(O)CH₂N(CH₃)₂, or —S(O)₂CH₃; or R² and R³ are taken together with the nitrogen atom to which they are attached to form a 6-membered heterocyclyl optionally containing one additional heteroatom selected from the group consisting of N and O, wherein the heterocyclyl is optionally substituted by 1-5 R⁴ groups. In some embodiments, R² and R³ are taken together with the nitrogen atom to which they are attached to form

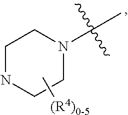

wherein the nitrogen atom at the 4-position of the ring is bound to H when not substituted by R⁴.

In some embodiments, each R⁴, where present, is independently: halogen, —CN, C₁-C₃ alkyl, C₁-C₃ haloalkyl, C₁-C₃ alkyl-OH, —N(C₁-C₃ alkyl)₂, —C(O)(C₁-C₃ alkyl), or hydroxyl; taken together with another R⁴ group and the carbon atom or atoms to which they are attached to form a spiro or fused 4- to 6-membered heterocyclyl containing 1-2 heteroatoms selected from the group consisting of N, O, and S; or taken together with another R⁴ group attached to the same ring atom to form an oxo group. In some embodiments, each R⁴ is independently: Cl, F, —CN, —CH₃, —N(CH₃)₂, —C(O)CH₃, or hydroxyl; taken together with another R⁴ group and the carbon atom or atoms to which they are attached to form a spiro or fused 4- to 6-membered heterocyclyl containing 1-2 heteroatoms selected from the group consisting of N, O, and S; or taken together with another R⁴ group attached to the same ring atom to form an oxo group. In some embodiments, each R⁴ is —CH₃. In some embodiments, two R⁴ groups attached to the same ring atom are taken together to form an oxo group. In some embodiments, two R⁴ groups attached to the same ring atom are taken together to form an oxo group and one additional R⁴ group is —CH₃.

In some embodiments, R⁵ is H, C₁-C₃ alkyl, or C₃-C₅ cycloalkyl. In some embodiments, R⁵ is H, —CH₃, or cyclopropyl. In some embodiments, R⁵ is H.

In some embodiments, R⁶ is H, halogen, C₁-C₃ alkyl, C₁-C₃ haloalkyl, or C₁-C₃ alkyl-OH. In some embodiments, R⁶ is H, Cl, —CH₃, —CF₃, or —CH₂OH. In some embodiments, R⁶ is H or —CH₃.

Also provided herein is a compound which is selected from the compounds in Table 1, or a pharmaceutically acceptable salt thereof.

In another aspect, provided herein is a pharmaceutical composition comprising any compound disclosed herein, or a pharmaceutically acceptable salt thereof, and at least one pharmaceutically acceptable carrier, diluent, or excipient.

In a further aspect, provided herein is a method of treating cancer in a patient in need thereof, comprising administering to the patient a therapeutically effective amount of any compound disclosed herein, or a pharmaceutically acceptable salt thereof, or a therapeutically effective amount of the pharmaceutical composition disclosed herein.

In some embodiments, the cancer is liver, colorectal, anal, breast, gastrointestinal, skin, stomach, esophageal, or pancreatic cancer. In some embodiments, the cancer originates from the liver or spreads to the liver. In some embodiments, the cancer is hepatocellular carcinoma (HCC). In some embodiments, the method further comprises administering one or more additional pharmaceutical agents. In some embodiments, the one or more additional pharmaceutical agents is selected from the group consisting of cabozantinib-S-malate, pembrolizumab, lenvatinib mesylate, sorafenib tosylate, nivolumab, ramucirumab, and regorafenib.

### DETAILED DESCRIPTION

### Definitions

As used herein, the following definitions shall apply unless otherwise indicated. Further, if any term or symbol used herein is not defined as set forth below, it shall have its ordinary meaning in the art.

“Comprising” is intended to mean that the compositions and methods include the recited elements, but not excluding others. “Consisting essentially of” when used to define compositions and methods, shall mean excluding other elements of any essential significance to the combination. For example, a composition consisting essentially of the elements as defined herein would not exclude other elements that do not materially affect the basic and novel characteristic(s) of the claimed invention. “Consisting of” shall mean excluding more than trace amount of, e.g., other ingredients and substantial method steps recited. Embodiments defined by each of these transition terms are within the scope of this invention. Thus, it is understood that aspects and embodiments described herein as “comprising” include “consisting of” and “consisting essentially of” embodiments.

“Effective amount” or dose of a compound or a composition refers to that amount of the compound, or a pharmaceutically acceptable salt thereof, or the composition that results in an intended result as desired based on the disclosure herein. Effective amounts can be determined by standard pharmaceutical procedures in cell cultures or experimental animals, e.g., and without limitation, by determining the LD₅₀ (the dose lethal to 50% of the population) and the ED₅₀ (the dose therapeutically effective in 50% of the population).

The term “excipient” as used herein means an inert or inactive substance that may be used in the production of a drug or pharmaceutical, such as a tablet containing a compound of the invention as an active ingredient. Various substances may be embraced by the term excipient, including without limitation any substance used as a binder, disintegrant, coating, compression/encapsulation aid, cream or lotion, lubricant, solutions for parenteral administration, materials for chewable tablets, sweetener or flavoring, suspending/gelling agent, or wet granulation agent. Binders include, e.g., carbomers, povidone, xanthan gum, etc.; coatings include, e.g., cellulose acetate phthalate, ethylcellulose, gellan gum, maltodextrin, enteric coatings, etc.; compression/encapsulation aids include, e.g., calcium carbonate, dextrose, fructose dc (dc=“directly compressible”), honey dc, lactose (anhydrate or monohydrate; optionally in combination with aspartame, cellulose, or microcrystalline cellulose), starch dc, sucrose, etc.; disintegrants include, e.g., croscarmellose sodium, gellan gum, sodium starch glycolate, etc.; creams or lotions include, e.g., maltodextrin, carrageenans, etc.; lubricants include, e.g., magnesium stearate, stearic acid, sodium stearyl fumarate, etc.; materials for chewable tablets include, e.g., dextrose, fructose dc, lactose (monohydrate, optionally in combination with aspartame or cellulose), etc.; suspending/gelling agents include, e.g., carrageenan, sodium starch glycolate, xanthan gum, etc.; sweeteners include, e.g., aspartame, dextrose, fructose dc, sorbitol, sucrose dc, etc.; and wet granulation agents include, e.g., calcium carbonate, maltodextrin, microcrystalline cellulose, etc.

“Patient” refers to mammals and includes humans and non-human mammals. Examples of patients include, but are not limited to mice, rats, hamsters, guinea pigs, pigs, rabbits, cats, dogs, goats, sheep, cows, and humans. In some embodiments, patient refers to a human.

“Pharmaceutically acceptable” refers to safe and non-toxic, preferably for in vivo, more preferably, for human administration.

“Pharmaceutically acceptable salt” refers to a salt that is pharmaceutically acceptable. A compound described herein may be administered as a pharmaceutically acceptable salt.

“Salt” refers to an ionic compound formed between an acid and a base. When the compound provided herein contains an acidic functionality, such salts include, without limitation, alkali metal, alkaline earth metal, and ammonium salts. As used herein, ammonium salts include, salts containing protonated nitrogen bases and alkylated nitrogen bases. Exemplary and non-limiting cations useful in pharmaceutically acceptable salts include Na, K, Rb, Cs, NH₄, Ca, Ba, imidazolium, and ammonium cations based on naturally occurring amino acids. When the compounds utilized herein contain basic functionality, such salts include, without limitation, salts of organic acids, such as carboxylic acids and sulfonic acids, and mineral acids, such as hydrogen halides, sulfuric acid, phosphoric acid, and the like. Exemplary and non-limiting anions useful in pharmaceutically acceptable salts include oxalate, maleate, acetate, propionate, succinate, tartrate, chloride, sulfate, bisulfate, mono-, di-, and tribasic phosphate, mesylate, tosylate, and the like.

“Therapeutically effective amount” or dose of a compound or a composition refers to that amount of the compound or the composition that results in reduction or inhibition of symptoms or a prolongation of survival in a patient. The results may require multiple doses of the compound or the composition.

As used herein, “treatment” or “treating” is an approach for obtaining beneficial or desired results including clinical results. For purposes of this invention, beneficial or desired results include, but are not limited to, one or more of the following: decreasing one more symptoms resulting from the disease or disorder, diminishing the extent of the disease or disorder, stabilizing the disease or disorder (e.g., preventing or delaying the worsening of the disease or disorder), delaying the occurrence or recurrence of the disease or disorder, delay or slowing the progression of the disease or disorder, ameliorating the disease or disorder state, providing a remission (whether partial or total) of the disease or disorder, decreasing the dose of one or more other medications required to treat the disease or disorder, enhancing the effect of another medication used to treat the disease or disorder, delaying the progression of the disease or disorder, increasing the quality of life, and/or prolonging survival of a patient. Also encompassed by “treatment” is a reduction of pathological consequence of the disease or disorder. The methods of the invention contemplate any one or more of these aspects of treatment.

An “isotopomer” of a compound is a compound in which one or more atoms of the compound have been replaced with isotopes of those same atoms. For example, where H has been replaced by D or T, or ¹²C has been replaced by ¹¹C or ¹⁴N has been replaced by ¹⁵N. For example, and without limitation, replacement of with D can in some instances lead to reduced rates of metabolism and therefore longer half-lives. Replacement of H with T can provide radioligands potentially useful in binding studies. Replacement of ¹²C with the short-lived isotope ¹¹C can provide ligands useful in Positron Emission Tomography (PET) scanning. Replacement of ¹⁴N with ¹⁵N provides compounds that can be detected/monitored by ¹⁵N NMR spectroscopy. For example, an isotopomer of a compound containing —CH₂CH₃ is that compound but containing —CD₂CD₃ instead of the —CH₂CH₃.

Unless a specific isotope of an element is indicated in a formula, the disclosure includes all isotopologues of the compounds disclosed herein, such as, for example, deuterated derivatives of the compounds (where H can be ²H, i.e., D). Isotopologues can have isotopic replacements at any or at all locations in a structure, or can have atoms present in natural abundance at any or all locations in a structure

“Stereoisomer” or “stereoisomers” refer to compounds that differ in the stereogenicity of the constituent atoms such as, without limitation, in the chirality of one or more stereocenters or related to the cis or trans configuration of a carbon-carbon or carbon-nitrogen double bond. Stereoisomers include enantiomers and diastereomers.

“Tautomer” refers to alternate forms of a compound that differ in the position of a proton, such as enol-keto and imine-enamine tautomers, or the tautomeric forms of heteroaryl groups containing a ring atom attached to both a ring —NH— moiety and a ring ═N— moiety such as pyrazoles, imidazoles, benzimidazoles, triazoles, and tetrazoles.

“Alkyl” refers to monovalent saturated aliphatic hydrocarbyl groups having from 1 to 12 carbon atoms, preferably from 1 to 10 carbon atoms, and more preferably from 1 to 6 carbon atoms. This term includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂—), and neopentyl ((CH₃)₃CCH₂—). Cₓ alkyl refers to an alkyl group having x number of carbon atoms.

“Alkenyl” refers to straight or branched monovalent hydrocarbyl groups having from 2 to 6 carbon atoms and preferably 2 to 4 carbon atoms and having at least 1 and preferably from 1 to 2 sites of vinyl (>C═C<) unsaturation. Such groups are exemplified, for example, by vinyl, allyl, and but-3-en-1-yl. Included within this term are the cis and trans isomers or mixtures of these isomers. Cₓ alkenyl refers to an alkenyl group having x number of carbon atoms.

“Alkynyl” refers to straight or branched monovalent hydrocarbyl groups having from 2 to 6 carbon atoms and preferably 2 to 3 carbon atoms and having at least 1 and preferably from 1 to 2 sites of acetylenic unsaturation. Examples of such alkynyl groups include acetylenyl (—C≡CH), and propargyl (—CH₂C≡CH). Cₓ alkynyl refers to an alkynyl group having x number of carbon atoms.

“Amino” refers to the group —NH₂.

“Aryl” or “Ar” refers to a monovalent aromatic carbocyclic group of from 6 to 14 carbon atoms having a single ring (e.g., phenyl (Ph)) or multiple condensed rings (e.g., naphthyl or anthryl) which condensed rings may or may not be aromatic (e.g., 2-benzoxazolinone, 2H-1,4-benzoxazin-3(4H)-one-7-yl, and the like) provided that the point of attachment is at an aromatic carbon atom. Examples of aryl groups include phenyl and naphthyl.

“Arylene” refers to a divalent aromatic carbocyclic group of from 6 to 14 carbon atoms having a single ring or multiple condensed rings which condensed rings may or may not be aromatic provided that the points of attachment are at aromatic carbon atoms. An example of an arylene group is phenylene.

“Carbonyl” refers to the divalent group —C(O)— which is equivalent to —C(═O)—.

“Cyano” refers to the group —C≡N.

“Cycloalkyl” refers to saturated or unsaturated but nonaromatic cyclic alkyl groups of from 3 to 10 carbon atoms, preferably from 3 to 8 carbon atoms, and more preferably from 3 to 6 carbon atoms, having single or multiple cyclic rings including fused, bridged, and spiro ring systems. Cₓ cycloalkyl refers to a cycloalkyl group having x number of ring carbon atoms. Examples of suitable cycloalkyl groups include, for instance, adamantyl, cyclopropyl, cyclobutyl, cyclopentyl, and cyclooctyl. One or more the rings can be aryl, heteroaryl, or heterocyclic provided that the point of attachment is through the non-aromatic, non-heterocyclic ring saturated carbocyclic ring.

“Halo” or “halogen” refers to fluoro, chloro, bromo and iodo and preferably is fluoro or chloro.

“Hydroxy” or “hydroxyl” refers to the group —OH.

“Heteroaryl” refers to an aromatic group of from 1 to 10 carbon atoms and 1 to 4 heteroatoms selected from the group consisting of oxygen, nitrogen and sulfur within the ring. Such heteroaryl groups can have a single ring (e.g., pyridinyl or furyl) or multiple condensed rings (e.g., indolizinyl or benzothienyl) wherein the condensed rings may or may not be aromatic and/or contain a heteroatom provided that the point of attachment is through an atom of the aromatic heteroaryl group. In one embodiment, the nitrogen and/or the sulfur ring atom(s) of the heteroaryl group are optionally oxidized to provide for the N-oxide (N→O), sulfinyl, or sulfonyl moieties. Examples of heteroaryls include 5 or 6 membered heteroaryls such as pyridinyl, pyrrolyl, thiophenyl, and furanyl. Other examples of heteroaryls include 9 or 10 membered heteroaryls, such as indolyl, quinolinyl, quinolonyl, isoquinolinyl, and isoquinolonyl.

“Heteroarylene” refers to a divalent aromatic group having from 1 to 10 carbon atoms and 1 to 4 heteroatoms selected from the group consisting of oxygen, nitrogen and sulfur within the ring. Heteroarylene groups can have a single ring or multiple condensed rings wherein the condensed rings may or may not be aromatic and/or contain a heteroatom provided that the point of attachment is through an atom of the aromatic heteroarylene group. In one embodiment, the nitrogen and/or the sulfur ring atom(s) of the heteroarylene group are optionally oxidized to provide for the N-oxide (N→O), sulfinyl, or sulfonyl moieties. An example of a heteroarylene group is pyridinylene.

“Heterocycle” or “heterocyclic” or “heterocycloalkyl” or “heterocyclyl” refers to a saturated or partially saturated, but not aromatic, group having from 1 to 10 ring carbon atoms, preferably from 1 to 8 carbon atoms, and more preferably from 1 to 6 carbon atoms, and from 1 to 4 ring heteroatoms, preferably from 1 to 3 heteroatoms, and more preferably from 1 to 2 heteroatoms selected from the group consisting of nitrogen, sulfur, or oxygen. Cₓ heterocycloalkyl refers to a heterocycloalkyl group having x number of ring atoms including the ring heteroatoms. Heterocycle encompasses single ring or multiple condensed rings, including fused bridged and spiro ring systems. In fused ring systems, one or more the rings can be cycloalkyl, aryl or heteroaryl provided that the point of attachment is through the non-aromatic ring. In one embodiment, the nitrogen and/or sulfur atom(s) of the heterocyclic group are optionally oxidized to provide for the N-oxide, sulfinyl, sulfonyl moieties.

Examples of heterocyclyl and heteroaryl include, but are not limited to, azetidinyl, pyrrolyl, imidazolyl, pyrazolyl, pyridyl, pyrazyl, pyrimidyl, pyridazyl, indolizyl, isoindolyl, indolyl, dihydroindolyl, indazolyl, purinyl, quinolizinyl, isoquinolinyl, quinolinyl, phthalazinyl, naphthylpyridinyl, quinoxalinyl, quinazolinyl, cinnolinyl, pteridinyl, carbazolyl, carbolinyl, phenanthridinyl, acridinyl, phenanthrolinyl, isothiazolyl, phenazinyl, isoxazolyl, phenoxazinyl, phenothiazinyl, imidazolidinyl, imidazolinyl, piperidinyl, piperazinyl, indolinyl, phthalimidyl, 1,2,3,4-tetrahydroisoquinolinyl, 4,5,6,7-tetrahydrobenzo[b]thiophenyl, thiazolyl, thiazolidinyl, thiophenyl, benzo[b]thiophenyl, morpholinyl, thiomorpholinyl (also referred to as thiamorpholinyl), 1,1-dioxothiomorpholinyl, piperidinyl, pyrrolidinyl, and tetrahydrofuranyl.

“Nitro” refers to the group —NO₂.

“Oxo” refers to the atom (═O) or (O).

“Spiro ring systems” refers to bicyclic ring systems that have a single ring carbon atom common to both rings.

“Vinyl” refers to unsaturated hydrocarbon radical —CH═CH₂, derived from ethylene.

The terms “optional” or “optionally” as used throughout the specification means that the subsequently described event or circumstance may but need not occur, and that the description includes instances where the event or circumstance occurs and instances in which it does not. For example, “the heterocyclyl group is optionally substituted by a halogen” means that the heterocyclyl group may but need not be substituted by a halogen, and the description includes situations where the heterocyclyl group is not substituted by a halogen and situations where the heterocyclyl group is substituted by a halogen.

Unless indicated otherwise, the nomenclature of substituents that are not explicitly defined herein are arrived at by naming the terminal portion of the functionality followed by the adjacent functionality toward the point of attachment. For example, the substituent “alkoxycarbonylalkyl” refers to the group (alkoxy)-C(O)-(alkyl)-.

“Optionally substituted”, unless otherwise specified, means that a group is unsubstituted or substituted by one or more (e.g., 1, 2, 3, 4, or 5) of the substituents listed for that group, in which the substituents may be the same or different. In one embodiment, an optionally substituted group is unsubstituted. In one embodiment, an optionally substituted group has one substituent. In another embodiment, an optionally substituted group has two substituents. In another embodiment, an optionally substituted group has three substituents. In another embodiment, an optionally substituted group has four substituents. In some embodiments, an optionally substituted group has 1 to 2, 1 to 3, 1 to 4, or 1 to 5 substituents. When multiple substituents are present, each substituent is independently chosen unless indicated otherwise. For example, each (C₁-C₃ alkyl) substituent on the group —N(C₁-C₃ alkyl)(C₁-C₃ alkyl) can be selected independently from the other, so as to generate groups such as —N(CH₃)(CH₂CH₃), etc.

In addition to the disclosure herein, the term “substituted”, when used to modify a specified group or radical, can also mean that one or more hydrogen atoms of the specified group or radical are each, independently of one another, replaced with the same or different substituent groups as defined herein. In some embodiments, a group that is substituted has 1, 2, 3, or 4 substituents, 1, 2, or 3 substituents, 1 or 2 substituents, or 1 substituent.

Substituents can be attached to any chemically possible location on the specified group or radical, unless indicated otherwise. Thus, —C₁-C₃ alkyl-OH includes, for example, —CH₂CH₂OH and —CH(OH)—CH₃.

It is understood that the above definitions are not intended to include impermissible substitution patterns (e.g., methyl substituted with 4 fluoro groups). Such impermissible substitution patterns are well known to the skilled artisan.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination. All combinations of the embodiments pertaining to the chemical groups represented by the variables are specifically embraced by the present invention and are disclosed herein just as if each and every combination was individually and explicitly disclosed, to the extent that such combinations embrace compounds that are stable compounds (i.e., compounds that can be isolated, characterized, and tested for biological activity). In addition, all subcombinations of the chemical groups listed in the embodiments describing such variables are also specifically embraced by the present invention and are disclosed herein just as if each and every such sub-combination of chemical groups was individually and explicitly disclosed herein.

### Compounds

In one aspect, provided is a compound of formula (I)

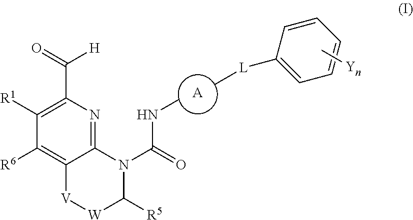

or a pharmaceutically acceptable salt thereof, wherein:

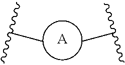

is 5- to 6-membered arylene or heteroarylene, each of which is optionally substituted by 1-4 halogen or C₁-C₆ alkyl groups;

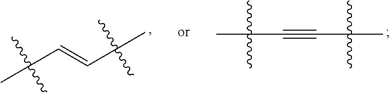

L is —OCH₂—, —CH₂O—, —CH₂CH₂—, each Y is independently halogen or —O(C₁-C₆ alkyl) optionally substituted by 1-5 groups independently selected from the group consisting of halogen, hydroxyl, —CN, and —NH₂, provided that when

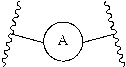

is 5-membered heteroarylene, then at least one Y, when present, is halogen;
n is 0-5;
V is CH₂, O, or CH(OH);
W is CH₂, CH₂CH₂, or a bond;
R¹ is H, halogen, hydroxyl, —CN, —NH₂, C₁-C₆ alkyl, C₁-C₆ haloalkyl, C₁-C₆ alkyl-OH, C₁-C₆ cycloalkyl, —CH₂NR²R³, —CH(CH₃)NR²R³, —O(C₁-C₆ alkyl), —CH₂CO₂H, —C(O)H, or 5- to 6-membered heterocyclyl or heteroaryl,
   wherein the heterocyclyl or heteroaryl contains 1-3 heteroatoms selected from the group consisting of N, O, and S, and each of which heterocyclyl or heteroaryl is optionally substituted by 1-5 groups independently selected from the group consisting of C₁-C₆ alkyl, C₁-C₆ haloalkyl, halogen, hydroxyl, —CN, —NH₂, oxo, and 4- to 6-membered heterocyclyl containing 1-3 heteroatoms selected from the group consisting of N, O, and S;
R² is H, C₁-C₆ alkyl, C₁-C₆ haloalkyl, C₁-C₆ alkyl-OH, or (C₁-C₆ alkyl)₂N—(C₁-C₆ alkylene);
R³ is H, C₁-C₆ alkyl, —C(O)(C₁-C₆ alkyl), C₁-C₆ haloalkyl, C₁-C₆ alkyl-OH, —C(O)CH₂OH, —C(O)CH₂O(C₁-C₆ alkyl), —C(O)CH₂N(C₁-C₆ alkyl)₂, or —S(O)₂(C₁-C₆ alkyl);
or R² and R³ are taken together with the nitrogen atom to which they are attached to form a 5- to 6-membered heterocyclyl optionally containing one additional heteroatom or heteroatom-containing moiety selected from the group consisting of N, N-oxide, O, and S, wherein the heterocyclyl is optionally substituted by 1-5 R⁴ groups;
each R⁴ is independently:
   halogen, —CN, C₁-C₆ alkyl, C₁-C₆ haloalkyl, C₁-C₆ alkyl-OH, —N(C₁-C₆ alkyl)₂, —C(O)(C₁-C₆ alkyl), or hydroxyl;
   taken together with another R⁴ group and the carbon atom or atoms to which they are attached to form a spiro or fused 4- to 6-membered heterocyclyl containing 1-3 heteroatoms selected from the group consisting of N, O, and S; or
   taken together with another R⁴ group attached to the same ring atom to form an oxo group;
R⁵ is H, C₁-C₆ alkyl, or C₁-C₆ cycloalkyl; and
R⁶ is H, halogen, C₁-C₆ alkyl, C₁-C₆ haloalkyl, or C₁-C₆ alkyl-OH.

In some embodiments,

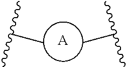

(i.e., the Ring A moiety) is 5- to 6-membered arylene or heteroarylene, each of which is optionally substituted by 1-4 halogen or C₁-C₆ alkyl groups. In some embodiments, the Ring A moiety is phenylene or 5- to 6-membered heteroarylene containing 1-3 nitrogen atoms, each of which phenylene or heteroarylene is optionally substituted by 1-2 halogen or C₁-C₃ alkyl groups. In some embodiments, the Ring A moiety is

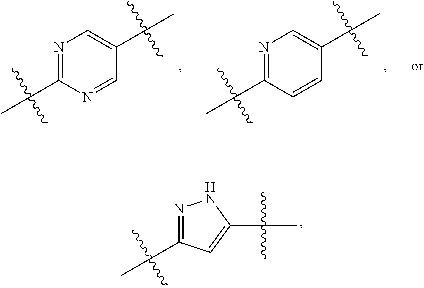 [SMILES: *c1cnc(*)nc1 ; *c1ccc(*)nc1 ; *c1cc(*)[nH]n1]

each of which is optionally substituted by 1-2 Cl or —CH₃ groups.

In some embodiments, the Ring A moiety is phenylene optionally substituted by 1-4 halogen or C₁-C₆ alkyl groups. In some embodiments, the Ring A moiety is phenylene optionally substituted by 1-2 halogen or C₁-C₃ alkyl groups. In some embodiments, the Ring A moiety is phenylene optionally substituted by 1-2 Cl or —CH₃ groups. In some embodiments, the Ring A moiety is unsubstituted phenylene.

In some embodiments, the Ring A moiety is 5- to 6-membered heteroarylene optionally substituted by 1-4 halogen or C₁-C₆ alkyl groups. In some embodiments, the 5- to 6-membered heteroarylene contains one, two or three heteroatoms selected from the group consisting of N, O, and S. In some embodiments, the 5- to 6-membered heteroarylene contains one, two or three nitrogen atoms. In some embodiments, the Ring A moiety is pyrrolylene, pyrazolylene, imidazolylene, triazolylene, pyridinylene, pyridazinylene, pyrimidinylene, pyrazinylene, or triazinylene. In some embodiments, the Ring A moiety is pyrazolylene, pyridinylene, or pyrimidinylene. In some embodiments, the Ring A moiety is

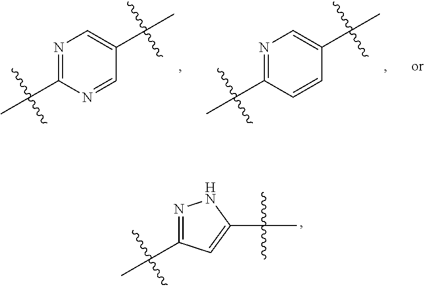 [SMILES: *c1cnc(*)nc1 ; *c1ccc(*)nc1 ; *c1cc(*)[nH]n1]

In some embodiments, the Ring A moiety is 5- to 6-membered heteroarylene, including any variation detailed herein, optionally substituted by 1-2 halogen or C₁-C₃ alkyl groups. In some embodiments, the Ring A moiety is 5- to 6-membered heteroarylene, including any variation detailed herein, optionally substituted by 1-2 Cl or —CH₃ groups. In some embodiments, the Ring A moiety is unsubstituted 5- to 6-membered heteroarylene, including any variation detailed herein.

In some embodiments, L is —OCH₂—, —CH₂O—, —CH₂CH₂—,

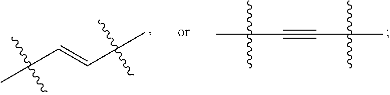

In some embodiments, L is —OCH₂—. In some embodiments, L is —CH₂O—. In some embodiments, L is —CH₂CH₂—. In some embodiments, L is

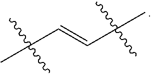 [SMILES: */C=C/*]

In some embodiments, L is

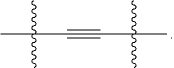 [SMILES: *C#C*]

In some embodiments, each Y, where present, is independently halogen or —O(C₁-C₆ alkyl) optionally substituted by 1-5 groups independently selected from the group consisting of halogen, hydroxyl, —CN, and —NH₂, provided that when the Ring A moiety is 5-membered heteroarylene, then at least one Y, when present, is halogen. In some embodiments, each Y, where present, is independently halogen or —O(C₁-C₃ alkyl) optionally substituted by 1-3 groups independently selected from the group consisting of halogen, hydroxyl, —CN, and —NH₂. In some embodiments, each Y, where present, is independently F, Cl, or —O(C₁-C₂ alkyl) optionally substituted by 1-2 groups independently selected from the group consisting of Cl, hydroxyl, —CN, and —NH₂. In some embodiments, each Y, where present, is independently F, Cl, or —OCH₃.

In some embodiments, each Y, where present, is independently halogen or —O(C₁-C₆ alkyl) optionally substituted by 1-5 groups independently selected from the group consisting of halogen, hydroxyl, —CN, and —NH₂, provided that when the Ring A moiety is 5-membered heteroarylene, then at least one Y, when present, is halogen. In some embodiments, each Y, where present, is independently halo, such as F, Cl or Br, or I, or —O(C₁-C₃ alkyl), such as —O(methyl), —O(ethyl), —O(n-propyl), or —O(isopropyl), optionally substituted by 1-3 groups independently selected from the group consisting of halogen, such as F, Cl, Br, or I, hydroxyl, —CN, and —NH₂. In some embodiments, each Y, where present, is independently F, Cl, or —O(C₁-C₂ alkyl), such as —O(methyl) or —O(ethyl), optionally substituted by 1-2 groups independently selected from the group consisting of Cl, hydroxyl, —CN, and —NH₂. In some embodiments, each Y, where present, is independently F, Cl, or —O(C₁-C₂ alkyl), such as —O(methyl) or —O(ethyl), optionally substituted by 1-2 groups independently selected from the group consisting of Cl, hydroxyl, —CN, and —NH₂. In some embodiments, each Y, where present, is independently F or —OCH₃. In some embodiments, each Y, where present, is independently Cl or —OCH₃.

In some embodiments, n is 0-5. In some embodiments, n is 0, 1, 2, 3, 4, or 5. In some embodiments, n is 1-5. In some embodiments, n is 0. In some embodiments, n is 1. In some embodiments, n is 2. In some embodiments, n is 3. In some embodiments, n is 4. In some embodiments, n is 5. In some embodiments, n is 2-4. In some embodiments, n is 2-5. In some embodiments, n is 3-5.

In some embodiments, the

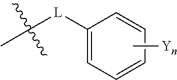

moiety is

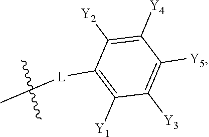

wherein Y¹, Y², Y³, Y⁴, and Y⁵ are independently hydrogen, halogen, or —O(C₁-C₆ alkyl) optionally substituted by 1-5 groups independently selected from the group consisting of halogen, hydroxyl, —CN, and —NH₂, provided that when the Ring A moiety is 5-membered heteroarylene, then (i) at least one of Y¹, Y², Y³, Y⁴, or Y⁵ is halogen, or (ii) Y¹, Y², Y³, Y⁴, and Y⁵ are each hydrogen. In some embodiments, Y¹ and Y² are independently hydrogen or halogen; and Y³, Y⁴, and Y⁵ are independently hydrogen, halogen, or —O(C₁-C₆ alkyl) optionally substituted by 1-5 groups independently selected from the group consisting of halogen, hydroxyl, —CN, and —NH₂, provided that when the Ring A moiety is 5-membered heteroarylene, then (i) at least one of Y¹, Y², Y³, Y⁴ or Y⁵ is halogen, or (ii) Y¹, Y², Y³, Y⁴, and Y⁵ are each hydrogen. In some embodiments, Y¹ and Y² are independently halogen; and Y³, Y⁴, and Y⁵ are independently hydrogen, halogen, or
—O(C₁-C₆ alkyl) optionally substituted by 1-5 groups independently selected from the group consisting of halogen, hydroxyl, —CN, and —NH₂. In some embodiments, Y¹ and Y² are independently halogen; Y³ and Y⁴ are independently hydrogen, halogen, or —O(C₁-C₆ alkyl) optionally substituted by 1-5 groups independently selected from the group consisting of halogen, hydroxyl, —CN, and —NH₂; and Y⁵ is hydrogen. In some embodiments, Y¹ and Y² are independently halogen; Y³ and Y⁴ are independently unsubstituted —O(C₁-C₃ alkyl); and Y⁵ is hydrogen. In some embodiments, Y¹ and Y² are each F; Y³ and Y⁴ are each —OCH₃; and Y⁵ is hydrogen. In some embodiments, Y¹ and Y² are independently Cl or F; Y³ and Y⁴ are each —OCH₃; and Y⁵ is hydrogen. In some embodiments, Y¹ and Y² are each Cl; Y³ and Y⁴ are each —OCH₃; and Y⁵ is hydrogen.

In some embodiments, V is CH₂, O, or CH(OH). In some embodiments, V is CH₂. In some embodiments, V is O. In some embodiments, V is CH(OH).

In some embodiments, W is CH₂, CH₂CH₂, or a bond. In some embodiments, W is CH₂. In some embodiments, W is CH₂CH₂. In some embodiments, W is a bond.

In some embodiments, R¹ is H, halogen, hydroxyl, —CN, —NH₂, C₁-C₆ alkyl, C₁-C₆ haloalkyl, C₁-C₆ alkyl-OH, C₁-C₆ cycloalkyl, —CH₂NR²R³, —CH(CH₃)NR²R³, —O(C₁-C₆ alkyl), —CH₂CO₂H, —C(O)H, or 5- to 6-membered heterocyclyl or heteroaryl, wherein the heterocyclyl or heteroaryl contains 1-3 heteroatoms selected from the group consisting of N, O, and S, and each of which heterocyclyl or heteroaryl is optionally substituted by 1-5 groups independently selected from the group consisting of C₁-C₆ alkyl, C₁-C₆ haloalkyl, halogen, hydroxyl, —CN, —NH₂, oxo, and 4- to 6-membered heterocyclyl containing 1-3 heteroatoms selected from the group consisting of N, O, and S. In some embodiments, R¹ is H, halogen, hydroxyl, —CN, —NH₂, C₁-C₃ alkyl, C₁-C₃ haloalkyl, C₁-C₃ alkyl-OH, C₁-C₆ cycloalkyl, —CH₂NR²R³, —CH(CH₃)NR²R³, —O(C₁-C₃ alkyl), —CH₂CO₂H, —C(O)H, or 5- to 6-membered heterocyclyl or heteroaryl, wherein the heterocyclyl or heteroaryl contains 1-3 heteroatoms selected from the group consisting of N, O, and S, and each of which heterocyclyl or heteroaryl is optionally substituted by 1-3 groups independently selected from the group consisting of C₁-C₃ alkyl, C₁-C₃ haloalkyl, halogen, hydroxyl, —CN, —NH₂, oxo, and 4- to 5-membered heterocyclyl containing 1-2 heteroatoms selected from the group consisting of N and O. In some embodiments, R¹ is H, Cl, —CH₃, hydroxyl, —CN, —NH₂, —CF₃, —CH₂OH, cyclohexyl, —CH₂NR²R³, —CH(CH₃)NR²R³, —OCH₃, —CH₂CO₂H, —C(O)H, or 5- to 6-membered heterocyclyl or heteroaryl, wherein the heterocyclyl or heteroaryl contains 1-2 heteroatoms selected from the group consisting of N, O, and S, and each of which heterocyclyl or heteroaryl is optionally substituted by 1-3 groups independently selected from the group consisting of —CH₃, —CF₃, Cl, F, hydroxyl, —CN, —NH₂, oxetanyl, and oxo. In some embodiments, R¹ is H. In some embodiments, R¹ is —CH₂NR²R³.

In some embodiments, R¹ is H, hydroxyl, —CN, —NH₂, —CH₂CO₂H, or —C(O)H.

In some embodiments, R¹ is halogen. In some embodiments, R¹ is F, Cl, Br, or I. In some embodiments, R¹ is F or Cl. In some embodiments, R¹ is F. In some embodiments, R¹ is Cl.

In some embodiments, R¹ is C₁-C₆ alkyl. In some embodiments, R¹ is C₁-C₃ alkyl, such as methyl, ethyl, n-propyl, or isopropyl. In some embodiments, R¹ is —CH₃.

In some embodiments, R¹ is C₁-C₆ haloalkyl. In some embodiments, R¹ is C₁-C₆ haloalkyl containing 1-7 halogen atoms. In some embodiments, R¹ is C₁-C₃ haloalkyl. In some embodiments, R¹ is C₁-C₃ haloalkyl containing 1-5 halogen atoms. In some embodiments, R¹ is C₁-C₃ haloalkyl containing 1-3 halogen atoms. In some embodiments, R¹ is C₁-C₂ haloalkyl. In some embodiments, R¹ is C₁-C₂ haloalkyl containing 1-3 halogen atoms. In some embodiments R¹ is C₁ haloalkyl. In some embodiments R¹ is C₁ haloalkyl containing 1-3 halogen atoms. In some embodiments, the halogen atoms are independently selected from the group consisting of F, Cl, and Br. In some embodiments, the halogen atoms are independently selected from the group consisting of F and Cl. In some embodiments, the halogen atoms are all F. In some embodiments, the halogen atoms are all Cl. In some embodiments, the halogen atoms are a combination of F and Cl. In some embodiments, R¹ is —CH₂F, —CHF₂, —CF₃, —CH₂Cl, —CHCl₂, —CCl₃, —CF₂Cl, —CFCl₂, or —CHFCl. In some embodiments, R¹ is —CF₃.

In some embodiments, R¹ is C₁-C₆ alkyl-OH. In some embodiments, R¹ is C₁-C₃ alkyl-OH, such as methyl-OH, ethyl-OH, n-propyl-OH, or isopropyl-OH. In some embodiments, R¹ is —CH₂OH, —CH₂CH₂OH, —CH(OH)CH₃, —CH₂CH₂CH₂OH, —CH₂CH(OH)CH₃, —CH(OH)CH₂CH₃, —CH(CH₃)CH₂OH, or —C(CH₃)₂OH. In some embodiments, R¹ is —CH₂OH.

In some embodiments, R¹ is C₁-C₆ cycloalkyl. In some embodiments, R¹ is C₃-C₅ cycloalkyl, such as cyclopropyl, cyclobutyl, or cyclopentyl. In some embodiments, R¹ is cyclopropyl.

In some embodiments, R¹ is —O(C₁-C₆ alkyl). In some embodiments, R¹ is —O(C₁-C₃ alkyl), such as —O(methyl), —O(ethyl), —O(n-propyl), or —O(isopropyl). In some embodiments, le is —OCH₃.

In some embodiments, R¹ is 5- to 6-membered heterocyclyl containing 1-3 heteroatoms selected from the group consisting of N, O, and S, wherein the heterocyclyl is optionally substituted by 1-5 groups independently selected from the group consisting of C₁-C₆ alkyl, C₁-C₆ haloalkyl, halogen, hydroxyl, —CN, —NH₂, oxo, and 4- to 6-membered heterocyclyl containing 1-3 heteroatoms selected from the group consisting of N, O, and S. In some embodiments, R¹ is 5- to 6-membered heterocyclyl containing 1-3 heteroatoms selected from the group consisting of N, O, and S. In some embodiments, R¹ is 5- to 6-membered heterocyclyl containing 1-2 heteroatoms selected from the group consisting of N, O, and S. In some embodiments, R¹ is 5- to 6-membered heterocyclyl containing one, two, or three nitrogen atoms. In some embodiments, R¹ is 5- to 6-membered heterocyclyl containing one nitrogen atom. In some embodiments, R¹ is 5- to 6-membered heterocyclyl containing one, two, or three oxygen atoms. In some embodiments, R¹ is 5- to 6-membered heterocyclyl containing one oxygen atom. In some embodiments, R¹ is 5- to 6-membered heterocyclyl containing one, two, or three sulfur atoms. In some embodiments, R¹ is 5- to 6-membered heterocyclyl containing one sulfur atom. In some embodiments, R¹ is 5- to 6-membered heterocyclyl containing one nitrogen atom and two oxygen atoms. In some embodiments, R¹ is 5- to 6-membered heterocyclyl containing two nitrogen atoms and one oxygen atom. In some embodiments, R¹ is 5- to 6-membered heterocyclyl containing one nitrogen atom and one oxygen atom. In some embodiments, R¹ is 5- to 6-membered heterocyclyl containing one nitrogen atom and two sulfur atoms. In some embodiments, R¹ is 5- to 6-membered heterocyclyl containing two nitrogen atoms and one sulfur atom. In some embodiments, R¹ is 5- to 6-membered heterocyclyl containing one nitrogen atom and one sulfur atom. In some embodiments, R¹ is 5- to 6-membered heterocyclyl containing one oxygen atom and two sulfur atoms. In some embodiments, R¹ is 5- to 6-membered heterocyclyl containing two oxygen atoms and one sulfur atom. In some embodiments, R¹ is 5- to 6-membered heterocyclyl containing one oxygen atom and one sulfur atom. In some embodiments, R¹ is

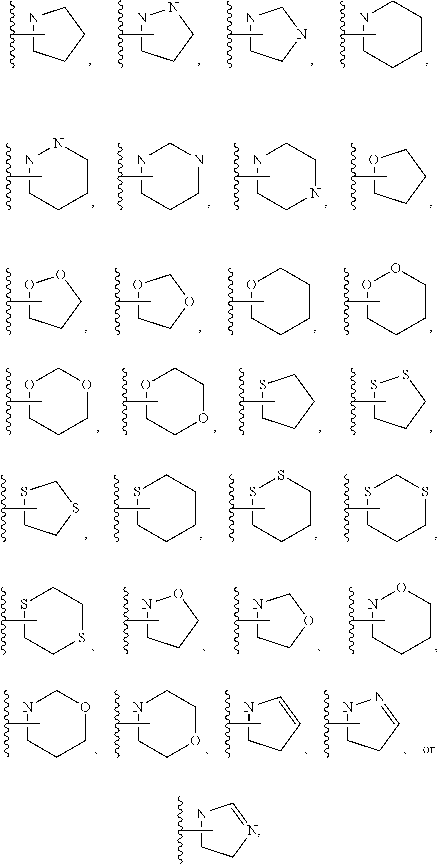

wherein the heteroatoms of the heterocyclyl groups, where applicable, are bound to H when not further substituted. In some embodiments, R¹ is 5- to 6-membered heterocyclyl, including any variation detailed herein, optionally substituted by 1-3 groups independently selected from the group consisting of C₁-C₃ alkyl (such as methyl, ethyl, n-propyl, or isopropyl), C₁-C₃ haloalkyl (such as halomethyl, haloethyl, halo-n-propyl, or haloisopropyl), halogen (such as F, Cl, Br, or I), hydroxyl, —CN, —NH₂, oxo, and 4- to 5-membered heterocyclyl containing 1-2 heteroatoms selected from the group consisting of N and O (such as oxetanyl, azetidinyl, tetrahydrofuranyl, pyrrolidinyl, dioxolanyl, pyrazolidinyl, imidazolidinyl, oxazolidinyl, or isoxazolidinyl). In some embodiments, R¹ is 5- to 6-membered heterocyclyl, including any variation detailed herein, optionally substituted by 1-3 groups independently selected from the group consisting of —CH₃, —CF₃, Cl, F, hydroxyl, —CN, —NH₂, oxetanyl, and oxo. In some embodiments, R¹ is 5- to 6-membered heterocyclyl, including any variation detailed herein, optionally substituted by 1-2 groups independently selected from the group consisting of —CH₃, —CF₃, Cl, F, hydroxyl, —CN, —NH₂, oxetanyl, and oxo. In some embodiments, R¹ is unsubstituted 5- to 6-membered heterocyclyl, including any variation detailed herein.

In some embodiments, R¹ is 5- to 6-membered heteroaryl containing 1-3 heteroatoms selected from the group consisting of N, O, and S, wherein the heteroaryl is optionally substituted by 1-5 groups independently selected from the group consisting of C₁-C₆ alkyl, C₁-C₆ haloalkyl, halogen, hydroxyl, —CN, —NH₂, oxo, and 4- to 6-membered heteroaryl containing 1-3 heteroatoms selected from the group consisting of N, O, and S. In some embodiments, R¹ is 5- to 6-membered heteroaryl containing 1-3 heteroatoms selected from the group consisting of N, O, and S. In some embodiments, R¹ is 5- to 6-membered heteroaryl containing 1-2 heteroatoms selected from the group consisting of N, O, and S. In some embodiments, R¹ is 5- to 6-membered heteroaryl containing one, two, or three nitrogen atoms. In some embodiments, R¹ is 5- to 6-membered heteroaryl containing one nitrogen atom. In some embodiments, R¹ is 5- to 6-membered heteroaryl containing one, two, or three oxygen atoms. In some embodiments, R¹ is 5- to 6-membered heteroaryl containing one oxygen atom. In some embodiments, R¹ is 5- to 6-membered heteroaryl containing one, two, or three sulfur atoms. In some embodiments, R¹ is 5- to 6-membered heteroaryl containing one sulfur atom. In some embodiments, R¹ is 5- to 6-membered heteroaryl containing one nitrogen atom and two oxygen atoms. In some embodiments, R¹ is 5- to 6-membered heteroaryl containing two nitrogen atoms and one oxygen atom. In some embodiments, R¹ is 5- to 6-membered heteroaryl containing one nitrogen atom and one oxygen atom. In some embodiments, R¹ is 5- to 6-membered heteroaryl containing one nitrogen atom and two sulfur atoms. In some embodiments, R¹ is 5- to 6-membered heteroaryl containing two nitrogen atoms and one sulfur atom. In some embodiments, R¹ is 5- to 6-membered heteroaryl containing one nitrogen atom and one sulfur atom. In some embodiments, R¹ is 5- to 6-membered heteroaryl containing one oxygen atom and two sulfur atoms. In some embodiments, R¹ is 5- to 6-membered heteroaryl containing two oxygen atoms and one sulfur atom. In some embodiments, R¹ is 5- to 6-membered heteroaryl containing one oxygen atom and one sulfur atom. In some embodiments, R¹ is pyrrolyl, pyrazolyl, imidazolyl, triazolyl, furanyl, thiophenyl, oxazolyl, thiazolyl, pyridinyl, pyridazinyl, pyrimidinyl, or pyrazinyl. In some embodiments, R¹ is 5- to 6-membered heteroaryl, including any variation detailed herein, optionally substituted by 1-3 groups independently selected from the group consisting of C₁-C₃ alkyl (such as methyl, ethyl, n-propyl, or isopropyl), C₁-C₃ haloalkyl (such as halomethyl, haloethyl, halo-n-propyl, or haloisopropyl), halogen (such as F, Cl, Br, or I), hydroxyl, —CN, —NH₂, oxo, and 4- to 5-membered heterocyclyl containing 1-2 heteroatoms selected from the group consisting of N and O (such as oxetanyl, azetidinyl, tetrahydrofuranyl, pyrrolidinyl, dioxolanyl, pyrazolidinyl, imidazolidinyl, oxazolidinyl, or isoxazolidinyl). In some embodiments, R¹ is 5- to 6-membered heteroaryl, including any variation detailed herein, optionally substituted by 1-3 groups independently selected from the group consisting of —CH₃, —CF₃, Cl, F, hydroxyl, —CN, —NH₂, oxetanyl, and oxo. In some embodiments, R¹ is 5- to 6-membered heteroaryl, including any variation detailed herein, optionally substituted by 1-2 groups independently selected from the group consisting of —CH₃, —CF₃, Cl, F, hydroxyl, —CN, —NH₂, oxetanyl, and oxo. In some embodiments, R¹¹ is unsubstituted 5- to 6-membered heteroaryl, including any variation detailed herein.

In some embodiments, R¹ is —CH₂NR²R³ or —CH(CH₃)NR²R³, wherein R² is H, C₁-C₆ alkyl, C₁-C₆ haloalkyl, C₁-C₆ alkyl-OH, or (C₁-C₆ alkyl)₂N—(C₁-C₆ alkylene), and R³ is H, C₁-C₆ alkyl, —C(O)(C₁-C₆ alkyl), C₁-C₆ haloalkyl, C₁-C₆ alkyl-OH, —C(O)CH₂OH, —C(O)CH₂O(C₁-C₆ alkyl), —C(O)CH₂N(C₁-C₆ alkyl)₂, or —S(O)₂(C₁-C₆ alkyl), or R² and R³ are taken together with the nitrogen atom to which they are attached to form a 5- to 6-membered heterocyclyl optionally containing one additional heteroatom or heteroatom-containing moiety selected from the group consisting of N, N-oxide, O, and S, wherein the heterocyclyl is optionally substituted by 1-5 R⁴ groups, wherein each R⁴ is independently: halogen, —CN, C₁-C₆ alkyl, C₁-C₆ haloalkyl, C₁-C₆ alkyl-OH, —N(C₁-C₆ alkyl)₂, —C(O)(C₁-C₆ alkyl), or hydroxyl; taken together with another R⁴ group and the carbon atom or atoms to which they are attached to form a spiro or fused 4- to 6-membered heterocyclyl containing 1-3 heteroatoms selected from the group consisting of N, O, and S; or taken together with another R⁴ group attached to the same ring atom to form an oxo group. In some embodiments, R¹ is —CH₂NR²R³. In some embodiments, R² is H, C₁-C₃ alkyl, C₁-C₃ haloalkyl, C₁-C₃ alkyl-OH, or (C₁-C₃ alkyl)₂N—(C₁-C₃ alkylene); and R³ is H, C₁-C₃ alkyl, C₁-C₃ haloalkyl, C₁-C₃ alkyl-OH, —C(O)(C₁-C₃ alkyl), —C(O)CH₂OH, —C(O)CH₂O(C₁-C₃ alkyl), —C(O)CH₂N(C₁-C₃ alkyl)₂, or —S(O)₂(C₁-C₃ alkyl); or R² and R³ are taken together with the nitrogen atom to which they are attached to form a 5- to 6-membered heterocyclyl optionally containing one additional heteroatom or heteroatom-containing moiety selected from the group consisting of N, N-oxide, O, and S, wherein the heterocyclyl is optionally substituted by 1-5 R⁴ groups. In some embodiments, R² is H, —CH₃, —CF₃, —CH₂OH, or (CH₃)₂N—CH₂—; and R³ is H, —CH₃, —CF₃, —CH₂OH, —C(O)(CH₃), —C(O)CH₂OH, —C(O)CH₂OCH₃, —C(O)CH₂N(CH₃)₂, or —S(O)₂CH₃; or R² and R³ are taken together with the nitrogen atom to which they are attached to form a 6-membered heterocyclyl optionally containing one additional heteroatom selected from the group consisting of N and O, wherein the heterocyclyl is optionally substituted by 1-5 R⁴ groups. In some embodiments, R² and R³ are taken together with the nitrogen atom to which they are attached to form

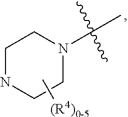

wherein the nitrogen at the 4-position of the ring is bound to H when not substituted by R⁴.

In some embodiments, R¹ is —CH₂NR²R³ or —CH(CH₃)NR²R³.

In some embodiments, R² is H, C₁-C₆ alkyl, C₁-C₆ haloalkyl, C₁-C₆ alkyl-OH, or (C₁-C₆ alkyl)₂N—(C₁-C₆ alkylene).

In some embodiments, R² is H.

In some embodiments, R² is C₁-C₆ alkyl. In some embodiments, R² is C₁-C₃ alkyl, such as methyl, ethyl, n-propyl, or isopropyl. In some embodiments, R² is —CH₃.

In some embodiments, R² is C₁-C₆ haloalkyl. In some embodiments, R² is C₁-C₆ haloalkyl containing 1-7 halogen atoms. In some embodiments, R² is C₁-C₃ haloalkyl. In some embodiments, R² is C₁-C₃ haloalkyl containing 1-5 halogen atoms. In some embodiments, R² is C₁-C₃ haloalkyl containing 1-3 halogen atoms. In some embodiments, R² is C₁-C₂ haloalkyl. In some embodiments, R² is C₁-C₂ haloalkyl containing 1-3 halogen atoms. In some embodiments R² is C₁ haloalkyl. In some embodiments R² is C₁ haloalkyl containing 1-3 halogen atoms. In some embodiments, the halogen atoms are independently selected from the group consisting of F, Cl, and Br. In some embodiments, the halogen atoms are independently selected from the group consisting of F and Cl. In some embodiments, the halogen atoms are all F. In some embodiments, the halogen atoms are all Cl. In some embodiments, the halogen atoms are a combination of F and Cl. In some embodiments, R² is —CH₂F, —CHF₂, —CF₃, —CH₂Cl, —CHCl₂, —CCl₃, —CF₂Cl, —CFCl₂, or —CHFCl. In some embodiments, R² is —CF₃.

In some embodiments, R² is C₁-C₆ alkyl-OH. In some embodiments, R² is C₁-C₃ alkyl-OH, such as methyl-OH, ethyl-OH, n-propyl-OH, or isopropyl-OH. In some embodiments, R² is —CH₂OH, —CH₂CH₂OH, —CH(OH)CH₃, —CH₂CH₂CH₂OH, —CH₂CH(OH)CH₃, —CH(OH)CH₂CH₃, —CH(CH₃)CH₂OH, or —C(CH₃)₂OH. In some embodiments, R² is —CH₂OH.

In some embodiments, R² is (C₁-C₆ alkyl)₂N—(C₁-C₆ alkylene). In some embodiments, R² is (C₁-C₆ alkyl)₂N—(C₁-C₃ alkylene). In some embodiments, R² is (C₁-C₃ alkyl)₂N—(C₁-C₆ alkylene). In some embodiments, R² is (C₁-C₃ alkyl)₂N—(C₁-C₃ alkylene). In some embodiments, R² is (C₁-C₂ alkyl)₂N—(C₁-C₂ alkylene). In some embodiments, R² is (C₁ alkyl)₂N—(C₁-C₂ alkylene). In some embodiments, R² is (C₁-C₂ alkyl)₂N—(C₁ alkylene). In some embodiments, R² is —CH₂CH₂—N(CH₂CH₃)₂, —CH₂CH₂—N(CH₂CH₃)CH₃, —CH₂CH₂—N(CH₃)₂, —CH₂—N(CH₂CH₃)₂, —CH₂—N(CH₂CH₃)CH₃, or —CH₂—N(CH₃)₂. In some embodiments, R² is —CH₂—N(CH₃)₂.

In some embodiments, R³ is H.

In some embodiments, R³ is C₁-C₆ alkyl. In some embodiments, R³ is C₁-C₃ alkyl, such as methyl, ethyl, n-propyl, or isopropyl. In some embodiments, R³ is —CH₃.

In some embodiments, R³ is —C(O)(C₁-C₆ alkyl). In some embodiments, R³ is —C(O)(C₁-C₃ alkyl), such as —C(O)CH₃, —C(O)CH₂CH₃, —C(O)CH₂CH₂CH₃, or —C(O)CH(CH₃)₂. In some embodiments, R³ is —C(O)CH₃.

In some embodiments, R³ is C₁-C₆ haloalkyl. In some embodiments, R³ is C₁-C₆ haloalkyl containing 1-7 halogen atoms. In some embodiments, R³ is C₁-C₃ haloalkyl. In some embodiments, R³ is C₁-C₃ haloalkyl containing 1-5 halogen atoms. In some embodiments, R³ is C₁-C₃ haloalkyl containing 1-3 halogen atoms. In some embodiments, R³ is C₁-C₂ haloalkyl. In some embodiments, R³ is C₁-C₂ haloalkyl containing 1-3 halogen atoms. In some embodiments R³ is C₁ haloalkyl. In some embodiments R³ is C₁ haloalkyl containing 1-3 halogen atoms. In some embodiments, the halogen atoms are independently selected from the group consisting of F, Cl, and Br. In some embodiments, the halogen atoms are independently selected from the group consisting of F and Cl. In some embodiments, the halogen atoms are all F. In some embodiments, the halogen atoms are all Cl. In some embodiments, the halogen atoms are a combination of F and Cl. In some embodiments, R³ is —CH₂F, —CHF₂, —CF₃, —CH₂Cl, —CHCl₂, —CCl₃, —CF₂Cl, —CFCl₂, or —CHFCl. In some embodiments, R³ is —CF₃.

In some embodiments, R³ is C₁-C₆ alkyl-OH. In some embodiments, R³ is C₁-C₃ alkyl-OH, such as methyl-OH, ethyl-OH, n-propyl-OH, or isopropyl-OH. In some embodiments, R³ is —CH₂OH, —CH₂CH₂OH, —CH(OH)CH₃, —CH₂CH₂CH₂OH, —CH₂CH(OH)CH₃, —CH(OH)CH₂CH₃, —CH(CH₃)CH₂OH, or —C(CH₃)₂OH. In some embodiments, R³ is —CH₂OH.

In some embodiments, R³ is —C(O)CH₂OH,

In some embodiments, R³ is —C(O)CH₂O(C₁-C₆ alkyl). In some embodiments, R³ is —C(O)CH₂O(C₁-C₃ alkyl), such as —C(O)CH₂OCH₃, —C(O)CH₂OCH₂CH₃, —C(O)CH₂OCH₂CH₂CH₃, or —C(O)CH₂OCH(CH₃)₂. In some embodiments, R³ is —C(O)CH₂OCH₃.

In some embodiments, R³ is —C(O)CH₂N(C₁-C₆ alkyl)₂. In some embodiments, R³ is —C(O)CH₂N(C₁-C₃ alkyl)₂, such as —C(O)CH₂N(CH₃)₂, —C(O)CH₂N(CH₂CH₃)₂, —C(O)CH₂N(CH₂CH₂CH₃)₂, —C(O)CH₂N(CH(CH₃)₂)₂, —C(O)CH₂N(CH₃)CH₂CH₃, —C(O)CH₂N(CH₃)CH₂CH₂CH₃, —C(O)CH₂N(CH₃)CH(CH₃)₂, —C(O)CH₂N(CH₂CH₃)CH₂CH₂CH₃, —C(O)CH₂N(CH₂CH₃)CH(CH₃)₂, or —C(O)CH₂N(CH₂CH₂CH₃)CH(CH₃)₂. In some embodiments, R³ is —C(O)CH₂N(CH₃)₂.

In some embodiments, R³ is —S(O)₂(C₁-C₆ alkyl). In some embodiments, R³ is —S(O)₂(C₁-C₃ alkyl), such as —S(O)₂CH₃, —S(O)₂CH₂CH₃, —S(O)₂CH₂CH₂CH₃, or —S(O)₂CH(CH₃)₂. In some embodiments, R³ is —S(O)₂CH₃.

In some embodiments, R² is H and R³ is C₁-C₆ alkyl. In some embodiments, R² is H and R³ is C₁-C₃ alkyl, such as methyl, ethyl, n-propyl, or isopropyl. In some embodiments, R² is H and R³ is —CH₃.

In some embodiments, R³ is H and R² is C₁-C₆ alkyl. In some embodiments, R³ is H and R² is C₁-C₃ alkyl, such as methyl, ethyl, n-propyl, or isopropyl. In some embodiments, R³ is H and R² is —CH₃.

In some embodiments, R² and R³ are each H.

In some embodiments, R² and R³ are taken together with the nitrogen atom to which they are attached to form a 5- to 6-membered heterocyclyl optionally containing one additional heteroatom or heteroatom-containing moiety selected from the group consisting of N, N-oxide, O, and S, wherein the heterocyclyl is optionally substituted by 1-5 R⁴ groups. In some embodiments, the 5- to 6-membered heterocyclyl optionally contains one additional heteroatom or heteroatom-containing moiety selected from the group consisting of N and O. In some embodiments, the 5- to 6-membered heterocyclyl contains two nitrogen atoms. In some embodiments, the 5- to 6-membered heterocyclyl contains one nitrogen atom and one oxygen atom. In some embodiments, the 5- to 6-membered heterocyclyl contains one nitrogen atom. In some embodiments, the 5- to 6-membered heterocyclyl is substituted by 5 R⁴ groups. In some embodiments, the 5- to 6-membered heterocyclyl is substituted by 4 R⁴ groups. In some embodiments, the 5- to 6-membered heterocyclyl is substituted by 3 R⁴ groups. In some embodiments, the 5- to 6-membered heterocyclyl is substituted by 2 R⁴ groups. In some embodiments, the 5- to 6-membered heterocyclyl is substituted by 1 R⁴ groups. In some embodiments, the 5- to 6-membered heterocyclyl is unsubstituted.

In some embodiments, R² and R³ are taken together with the nitrogen atom to which they are attached to form a 5-membered heterocyclyl, such as

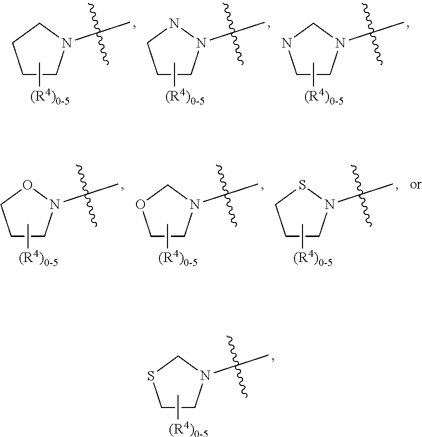

wherein the heteroatoms of the heterocyclyl, where applicable, are bound to H when not substituted by R⁴. In some embodiments, R² and R³ are taken together with the nitrogen atom to which they are attached to form a 6-membered heterocyclyl, such as

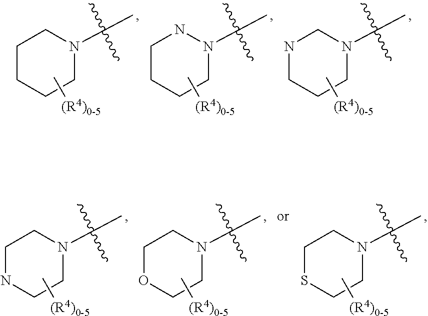

wherein the heteroatoms of the heterocyclyl, where applicable, are bound to H when not substituted by R⁴. In some embodiments, R² and R³ are taken together with the nitrogen atom to which they are attached to form

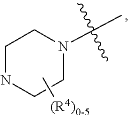

wherein the nitrogen atom at the 4-position of the ring is bound to H when not substituted by R⁴. In some embodiments, R² and R³ are taken together with the nitrogen atom to which they are attached to form unsubstituted

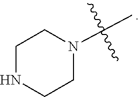 [SMILES: *N1CCNCC1]

In some embodiments, R² and R³ are taken together with the nitrogen atom to which they are attached to form

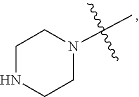 [SMILES: *N1CCNCC1]

which is substituted by 1-5 R⁴ groups.

In some embodiments, each R⁴ is independently: halogen, —CN, C₁-C₆ alkyl, C₁-C₆ haloalkyl, C₁-C₆ alkyl-OH, —N(C₁-C₆ alkyl)₂, —C(O)(C₁-C₆ alkyl), or hydroxyl; two R⁴ groups are taken together with the carbon atom or atoms to which they are attached to form a spiro or fused 4- to 6-membered heterocyclyl containing 1-3 heteroatoms selected from the group consisting of N, O, and S; or two R⁴ groups attached to the same ring atom are taken together to form an oxo group. In some embodiments, each R⁴, where present, is independently: halogen, —CN, C₁-C₃ alkyl, C₁-C₃ haloalkyl, C₁-C₃ alkyl-OH, —N(C₁-C₃ alkyl)₂, —C(O)(C₁-C₃ alkyl), or hydroxyl; taken together with another R⁴ group and the carbon atom or atoms to which they are attached to form a spiro or fused 4- to 6-membered heterocyclyl containing 1-2 heteroatoms selected from the group consisting of N, O, and S; or taken together with another R⁴ group attached to the same ring atom to form an oxo group. In some embodiments, each R⁴ is independently: Cl, F, —CN, —CH₃, —N(CH₃)₂, —C(O)CH₃, or hydroxyl; taken together with another R⁴ group and the carbon atom or atoms to which they are attached to form a spiro or fused 4- to 6-membered heterocyclyl containing 1-2 heteroatoms selected from the group consisting of N, O, and S; or taken together with another R⁴ group attached to the same ring atom to form an oxo group. In some embodiments, each R⁴ is —CH₃. In some embodiments, two R⁴ groups attached to the same ring atom are taken together to form an oxo group.

In some embodiments, R⁴ is halogen. In some embodiments, R⁴ is F, Cl, or Br. In some embodiments, R⁴ is F or Cl. In some embodiments, R⁴ is F. In some embodiments, R⁴ is Cl.

In some embodiments, R⁴ is —CN.

In some embodiments, R⁴ is C₁-C₆ alkyl. In some embodiments, R⁴ is C₁-C₃ alkyl, such as methyl, ethyl, n-propyl, or isopropyl. In some embodiments, R⁴ is —CH₃.

In some embodiments, R⁴ is C₁-C₆ haloalkyl. In some embodiments, R⁴ is C₁-C₆ haloalkyl containing 1-7 halogen atoms. In some embodiments, R⁴ is C₁-C₃ haloalkyl. In some embodiments, R⁴ is C₁-C₃ haloalkyl containing 1-5 halogen atoms. In some embodiments, R⁴ is C₁-C₃ haloalkyl containing 1-3 halogen atoms. In some embodiments, R⁴ is C₁-C₂ haloalkyl. In some embodiments, R⁴ is C₁-C₂ haloalkyl containing 1-3 halogen atoms. In some embodiments R⁴ is C₁ haloalkyl. In some embodiments R⁴ is C₁ haloalkyl containing 1-3 halogen atoms. In some embodiments, the halogen atoms are independently selected from the group consisting of F, Cl, and Br. In some embodiments, the halogen atoms are independently selected from the group consisting of F and Cl. In some embodiments, the halogen atoms are all F. In some embodiments, the halogen atoms are all Cl. In some embodiments, the halogen atoms are a combination of F and Cl. In some embodiments, R⁴ is —CH₂F, —CHF₂, —CF₃, —CH₂Cl, —CHCl₂, —CCl₃, —CF₂Cl, —CFCl₂, or —CHFCl. In some embodiments, R⁴ is —CF₃.

In some embodiments, R⁴ is C₁-C₆ alkyl-OH. In some embodiments, R⁴ is C₁-C₃ alkyl-OH, such as methyl-OH, ethyl-OH, n-propyl-OH, or isopropyl-OH. In some embodiments, R⁴ is —CH₂OH, —CH₂CH₂OH, —CH(OH)CH₃, —CH₂CH₂CH₂OH, —CH₂CH(OH)CH₃, —CH(OH)CH₂CH₃, —CH(CH₃)CH₂OH, or —C(CH₃)₂OH. In some embodiments, R⁴ is —CH₂OH.

In some embodiments, R⁴ is —N(C₁-C₆ alkyl)2. In some embodiments, R⁴ is —N(C₁-C₃ alkyl)₂. In some embodiments, R⁴ is —N(C₁-C₂ alkyl)₂. In some embodiments, R⁴ is —N(CH₃)₂, —N(CH₂CH₃)₂, —N(CH₂CH₂CH₃)₂, —N(CH(CH₃)₂)₂, —N(CH₃)CH₂CH₃, —N(CH₃)CH₂CH₂CH₃, —N(CH₃)CH(CH₃)₂, —N(CH₂CH₃)CH₂CH₂CH₃, —N(CH₂CH₃)CH(CH₃)₂, or —N(CH₂CH₂CH₃)CH(CH₃)₂. In some embodiments, R⁴ is —N(CH₃)₂.

In some embodiments, R⁴ is —C(O)(C₁-C₆ alkyl). In some embodiments, R⁴ is —C(O)(C₁-C₃ alkyl), such as —C(O)CH₃, —C(O)CH₂CH₃, —C(O)CH₂CH₂CH₃, or —C(O)CH(CH₃)₂. In some embodiments, R⁴ is —C(O)CH₃.

In some embodiments, R⁴ is hydroxyl.

In some embodiments, two R⁴ groups are taken together with the carbon atom or atoms to which they are attached to form a spiro or fused 4- to 6-membered heterocyclyl containing 1-3 heteroatoms selected from the group consisting of N, O, and S. In some embodiments, the spiro or fused 4- to 6-membered heterocyclyl contains 1-2 heteroatoms selected from the group consisting of N, O, and S. In some embodiments, the spiro or fused 4- to 6-membered heterocyclyl contains two nitrogen atoms. In some embodiments, the spiro or fused 4- to 6-membered heterocyclyl contains one nitrogen atom. In some embodiments, the spiro or fused 4- to 6-membered heterocyclyl contains two oxygen atoms. In some embodiments, the spiro or fused 4- to 6-membered heterocyclyl contains one oxygen atom. In some embodiments, the spiro or fused 4- to 6-membered heterocyclyl contains two sulfur atoms. In some embodiments, the spiro or fused 4- to 6-membered heterocyclyl contains one sulfur atom. In some embodiments, the spiro or fused 4- to 6-membered heterocyclyl contains one nitrogen atom and one oxygen atom. In some embodiments, the spiro or fused 4- to 6-membered heterocyclyl contains one nitrogen atom and one sulfur atom. In some embodiments, the spiro or fused 4- to 6-membered heterocyclyl contains one oxygen atom and one sulfur atom. In some embodiments, the spiro or fused 4- to 6-membered heterocyclyl.

In some embodiments, two R⁴ groups attached to the same ring atom are taken together to form an oxo group.

In some embodiments, two R⁴ groups attached to the same ring atom are taken together to form an oxo group and one additional R⁴ group is C₁-C₆ alkyl. In some embodiments, two R⁴ groups attached to the same ring atom are taken together to form an oxo group and one additional R⁴ group is C₁-C₃ alkyl. In some embodiments, two R⁴ groups attached to the same ring atom are taken together to form an oxo group and one additional R⁴ group is —CH₃.

In some embodiments, R⁵ is H, C₁-C₆ alkyl, or C₁-C₆ cycloalkyl. In some embodiments, R⁵ is H, C₁-C₃ alkyl, or C₃-C₅ cycloalkyl. In some embodiments, R⁵ is H, —CH₃, or cyclopropyl.

In some embodiments, R⁵ is H.

In some embodiments, R⁵ is C₁-C₆ alkyl. In some embodiments, R⁵ is C₁-C₃ alkyl, such as methyl, ethyl, n-propyl, or isopropyl. In some embodiments, R⁵ is —CH₃.

In some embodiments, R⁵ is C₁-C₆ cycloalkyl. In some embodiments, R⁵ is C₃-C₅ cycloalkyl, such as cyclopropyl, cyclobutyl, or cyclopentyl. In some embodiments, R⁵ is cyclopropyl.

In some embodiments, R⁶ is H, halogen, C₁-C₆ alkyl, C₁-C₆ haloalkyl, or C₁-C₆ alkyl-OH. In some embodiments, R⁶ is H, halogen, C₁-C₃ alkyl, C₁-C₃ haloalkyl, or C₁-C₃ alkyl-OH. In some embodiments, R⁶ is H, Cl, —CH₃, —CF₃, or —CH₂OH. In some embodiments, R⁶ is H or —CH₃.

In some embodiments, R⁶ is H.

In some embodiments, R⁶ is halogen. In some embodiments, R⁶ is F, Cl, Br, or I. In some embodiments, R⁶ is F. In some embodiments, R⁶ is Cl.

In some embodiments, R⁶ is C₁-C₆ alkyl. In some embodiments, R⁶ is C₁-C₃ alkyl, such as methyl, ethyl, n-propyl, or isopropyl. In some embodiments, R⁶ is —CH₃.

In some embodiments, R⁶ is C₁-C₆ haloalkyl. In some embodiments, R⁶ is C₁-C₆ haloalkyl containing 1-7 halogen atoms. In some embodiments, R⁶ is C₁-C₃ haloalkyl. In some embodiments, R⁶ is C₁-C₃ haloalkyl containing 1-5 halogen atoms. In some embodiments, R¹ is C₁-C₃ haloalkyl containing 1-3 halogen atoms. In some embodiments, R⁶ is C₁-C₂ haloalkyl. In some embodiments, R⁶ is C₁-C₂ haloalkyl containing 1-3 halogen atoms. In some embodiments R⁶ is C₁ haloalkyl. In some embodiments R⁶ is C₁ haloalkyl containing 1-3 halogen atoms. In some embodiments, the halogen atoms are independently selected from the group consisting of F, Cl, and Br. In some embodiments, the halogen atoms are independently selected from the group consisting of F and Cl. In some embodiments, the halogen atoms are all F. In some embodiments, the halogen atoms are all Cl. In some embodiments, the halogen atoms are a combination of F and Cl. In some embodiments, R⁶ is —CH₂F, —CHF₂, —CF₃, —CH₂Cl, —CHCl₂, —CCl₂, —CF₂Cl, —CFCl₂, or —CHFCl. In some embodiments, R⁶ is —CF₃.

In some embodiments, R⁶ is C₁-C₆ alkyl-OH. In some embodiments, R⁶ is C₁-C₃ alkyl-OH, such as methyl-OH, ethyl-OH, n-propyl-OH, or isopropyl-OH. In some embodiments, R⁶ is —CH₂OH, —CH₂CH₂OH, —CH(OH)CH₃, —CH₂CH₂CH₂OH, —CH₂CH(OH)CH₃, —CH(OH)CH₂CH₃, —CH(CH₃)CH₂OH, or —C(CH₃)₂OH. In some embodiments, R⁶ is —CH₂OH.

In some embodiments, the compound provided is of formula (I-a):

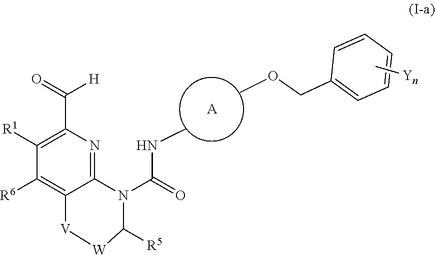

or a pharmaceutically acceptable salt thereof,
wherein the Ring A moiety, Y, n, V, W, R¹, R⁵, and R⁶ are as defined herein for any embodiment or variation of a compound of formula (I). In some embodiments, the Ring A moiety is phenylene optionally substituted by 1-2 halogen or C₁-C₃ alkyl groups. In some embodiments, the Ring A moiety is 5-membered heteroarylene containing 1-2 nitrogen atoms, wherein the heteroarylene is optionally substituted by 1-2 halogen or C₁-C₃ alkyl groups. In some embodiments, the Ring A moiety is unsubstituted 5-membered heteroarylene containing 2 nitrogen atoms. In some embodiments, the Ring A moiety is 6-membered heteroarylene containing 1-2 nitrogen atoms, wherein the heteroarylene is optionally substituted by 1-2 halogen or C₁-C₃ alkyl groups. In some embodiments, the Ring A moiety is unsubstituted 6-membered heteroarylene containing 1-2 nitrogen atoms. In some embodiments, the Ring A moiety is pyrazolylene, pyridinylene, or pyrimidinylene optionally substituted by 1-2 halogen or C₁-C₃ alkyl groups. In some embodiments, the Ring A moiety is

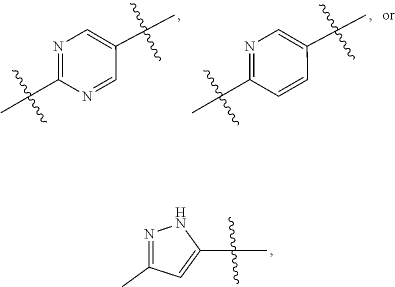

each of which is unsubstituted. In some embodiments, n is 4. In some embodiments, each Y is independently halogen or —O(C₁-C₆ alkyl) optionally substituted by 1-5 groups independently selected from the group consisting of halogen, hydroxyl, —CN, and —NH₂. In some embodiments, each Y is independently F, Cl, or unsubstituted —O(C₁-C₃ alkyl). In some embodiments, each Y is independently F, Cl, or —OCH₃. In some embodiments, n is 4, two Y groups are F, and two Y groups are —OCH₃. In some embodiments, n is 4, two Y groups are Cl, and two Y groups are —OCH₃. In some embodiments, V is CH₂. In some embodiments, W is CH₂. In some embodiments, R¹ is H or —CH₂NR²R³, wherein R² and R³ are taken together with the nitrogen atom to which they are attached to form a 5- to 6-membered heterocyclyl optionally containing one additional heteroatom or heteroatom-containing moiety selected from the group consisting of N, N-oxide, O, and S, wherein the heterocyclyl is optionally substituted by 1-5 R⁴ groups. In some embodiments, R¹ is H. In some embodiments, R¹ is CH₂NR²R³, wherein R² and R³ are taken together with the nitrogen atom to which they are attached to form a 6-membered heterocyclyl optionally containing one additional nitrogen atom, wherein the heterocyclyl is optionally substituted by 1-5 R⁴ groups. In some embodiments, R¹ is CH₂NR²R³, wherein R² and R³ are taken together with the nitrogen atom to which they are attached to form piperazinyl optionally substituted by 1-5 R⁴ groups. In some embodiments, R¹ is CH₂NR²R³, wherein R² and R³ are taken together with the nitrogen atom to which they are attached to form

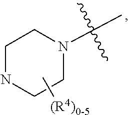

wherein the nitrogen atom at the 4-position of the ring is bound to H when not substituted by R⁴. In some embodiments, R⁴ is C₁-C₆ alkyl. In some embodiments, R⁴ is C₁-C₃ alkyl. In some embodiments, R⁴ is —CH₃. In some embodiments, two R⁴ groups attached to the same ring atom are taken together to form an oxo group. In some embodiments, two R⁴ groups attached to the same ring atom are taken together to form an oxo group and one additional R⁴ group is —CH₃. In some embodiments, R⁵ is H. In some embodiments, R⁶ is H or C₁-C₆ alkyl. In some embodiments, R⁶ is H. In some embodiments, R⁶ is C₁-C₃ alkyl. In some embodiments, R⁶ is —CH₃.

In some embodiments, the compound provided is of formula (I-b):

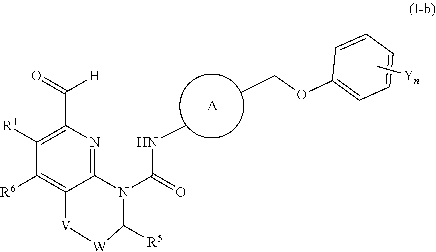

or a pharmaceutically acceptable salt thereof,
wherein the Ring A moiety, Y, n, V, W, R¹, R⁵, and R⁶ are as defined herein for any embodiment or variation of a compound of formula (I). In some embodiments, the Ring A moiety is phenylene optionally substituted by 1-2 halogen or C₁-C₃ alkyl groups. In some embodiments, the Ring A moiety is 5-membered heteroarylene containing 1-2 nitrogen atoms, wherein the heteroarylene is optionally substituted by 1-2 halogen or C₁-C₃ alkyl groups. In some embodiments, the Ring A moiety is 6-membered heteroarylene containing 1-2 nitrogen atoms, wherein the heteroarylene is optionally substituted by 1-2 halogen or C₁-C₃ alkyl groups. In some embodiments, n is 4. In some embodiments, each Y is independently halogen or —O(C₁-C₆ alkyl) optionally substituted by 1-5 groups independently selected from the group consisting of halogen, hydroxyl, —CN, and —NH₂. In some embodiments, each Y is independently F, Cl, or unsubstituted —O(C₁-C₃ alkyl). In some embodiments, each Y is independently F, Cl, or —OCH₃. In some embodiments, n is 4, two Y groups are F, and two Y groups are —OCH₃. In some embodiments, n is 4, two Y groups are Cl, and two Y groups are —OCH₃. In some embodiments, V is CH₂. In some embodiments, W is CH₂. In some embodiments, R¹ is H. In some embodiments, R⁵ is H. In some embodiments, R⁶ is H.

In some embodiments, the compound provided is of formula (I-c):

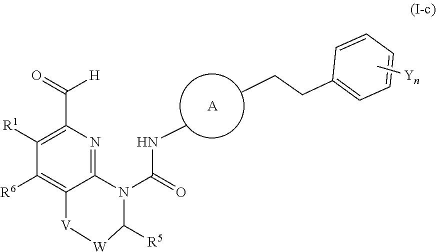

or a pharmaceutically acceptable salt thereof,
wherein the Ring A moiety, Y, n, V, W, R¹, R⁵, and R⁶ are as defined herein for any embodiment or variation of a compound of formula (I). In some embodiments, the Ring A moiety is 6-membered heteroarylene containing 1-2 nitrogen atoms, wherein the heteroarylene is optionally substituted by 1-2 halogen or C₁-C₃ alkyl groups. In some embodiments, the Ring A moiety is unsubstituted 6-membered heteroarylene containing 2 nitrogen atoms. In some embodiments, the Ring A moiety is unsubstituted pyrimidinylene. In some embodiments, n is 4. In some embodiments, each Y is independently halogen or —O(C₁-C₆ alkyl) optionally substituted by 1-5 groups independently selected from the group consisting of halogen, hydroxyl, —CN, and —NH₂. In some embodiments, each Y is independently F, Cl, or unsubstituted —O(C₁-C₃ alkyl). In some embodiments, each Y is independently F, Cl, or —OCH₃. In some embodiments, n is 4, two Y groups are F, and two Y groups are —OCH₃. In some embodiments, n is 4, two Y groups are Cl, and two Y groups are —OCH₃. In some embodiments, V is CH₂. In some embodiments, W is CH₂. In some embodiments, R¹ is H. In some embodiments, R⁵ is H. In some embodiments, R⁶ is H.

In some embodiments, the compound provided is of formula (I-d):

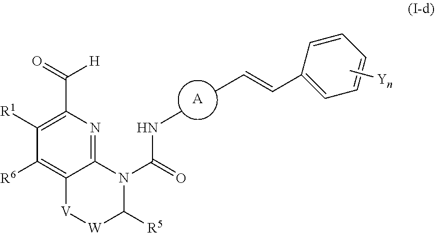

or a pharmaceutically acceptable salt thereof,
wherein the Ring A moiety, Y, n, V, W, R¹, R⁵, and R⁶ are as defined herein for any embodiment or variation of a compound of formula (I). In some embodiments, the Ring A moiety is 6-membered heteroarylene containing 1-2 nitrogen atoms, wherein the heteroarylene is optionally substituted by 1-2 halogen or C₁-C₃ alkyl groups. In some embodiments, the Ring A moiety is unsubstituted 6-membered heteroarylene containing 2 nitrogen atoms. In some embodiments, the Ring A moiety is unsubstituted pyrimidinylene. In some embodiments, n is 4. In some embodiments, each Y is independently halogen or —O(C₁-C₆ alkyl) optionally substituted by 1-5 groups independently selected from the group consisting of halogen, hydroxyl, —CN, and —NH₂. In some embodiments, each Y is independently F, Cl, or unsubstituted —O(C₁-C₃ alkyl). In some embodiments, each Y is independently F, Cl, or —OCH₃. In some embodiments, n is 4, two Y groups are F, and two Y groups are —OCH₃. In some embodiments, n is 4, two Y groups are Cl, and two Y groups are —OCH₃. In some embodiments, V is CH₂. In some embodiments, W is CH₂. In some embodiments, R¹ is H. In some embodiments, R⁵ is H. In some embodiments, R⁶ is H.

In some embodiments, the compound provided is of formula (I-e):

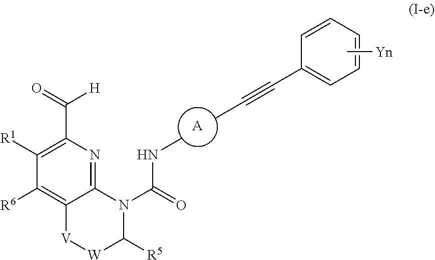

or a pharmaceutically acceptable salt thereof,
wherein the Ring A moiety, Y, n, V, W, R¹, R⁵, and R⁶ are as defined herein for any embodiment or variation of a compound of formula (I). In some embodiments, the Ring A moiety is 6-membered heteroarylene containing 1-2 nitrogen atoms, wherein the heteroarylene is optionally substituted by 1-2 halogen or C₁-C₃ alkyl groups. In some embodiments, the Ring A moiety is unsubstituted 6-membered heteroarylene containing 2 nitrogen atoms. In some embodiments, the Ring A moiety is unsubstituted pyrimidinylene. In some embodiments, n is 4. In some embodiments, each Y is independently halogen or —O(C₁-C₆ alkyl) optionally substituted by 1-5 groups independently selected from the group consisting of halogen, hydroxyl, —CN, and —NH₂. In some embodiments, each Y is independently F, Cl, or unsubstituted —O(C₁-C₃ alkyl). In some embodiments, each Y is independently F, Cl, or —OCH₃. In some embodiments, n is 4, two Y groups are F, and two Y groups are —OCH₃. In some embodiments, n is 4, two Y groups are Cl, and two Y groups are —OCH₃. In some embodiments, V is CH₂. In some embodiments, W is CH₂. In some embodiments, R¹ is H. In some embodiments, R⁵ is H. In some embodiments, R⁶ is H.

In some embodiments, the compound provided is of formula (II):

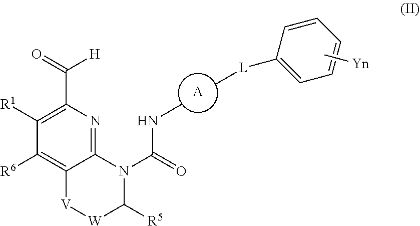

or a pharmaceutically acceptable salt thereof,
wherein L, Y, n, V, W, R¹, R⁵, and R⁶ are as defined herein for any embodiment or variation of a compound of formula (I), and

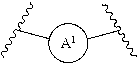

(i.e., the Ring A¹ moiety) is phenylene optionally substituted by 1-4 halogen or C₁-C₆ alkyl groups. In some embodiments, the Ring A¹ moiety is 1,3-phenylene optionally substituted by 1-2 halogen or C₁-C₃ alkyl groups. In some embodiments, the Ring A¹ moiety is unsubstituted 1,3-phenylene. In some embodiments, the Ring A¹ moiety is 1,4-phenylene optionally substituted by 1-2 halogen or C₁-C₃ alkyl groups. In some embodiments, the Ring A¹ moiety is unsubstituted 1,4-phenylene. In some embodiments, n is 4. In some embodiments, each Y is independently halogen or —O(C₁-C₆ alkyl) optionally substituted by 1-5 groups independently selected from the group consisting of halogen, hydroxyl, —CN, and —NH₂. In some embodiments, each Y is independently F, Cl, or unsubstituted —O(C₁-C₃ alkyl). In some embodiments, each Y is independently F, Cl, or —OCH₃. In some embodiments, n is 4, two Y groups are F, and two Y groups are —OCH₃. In some embodiments, n is 4, two Y groups are Cl, and two Y groups are —OCH₃. In some embodiments, V is CH₂. In some embodiments, W is CH₂. In some embodiments, R¹ is H. In some embodiments, R⁵ is H. In some embodiments, R⁶ is H or R⁶ is C₁-C₆ alkyl. In some embodiments, R⁶ is H.

In some embodiments, the compound provided is of formula (II-a):

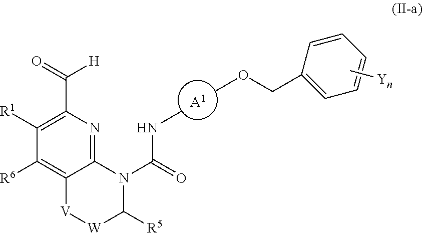

or a pharmaceutically acceptable salt thereof,
wherein Y, n, V, W, R¹, R⁵, and R⁶ are as defined herein for any embodiment or variation of a compound of formula (I), and the Ring A¹ moiety is as defined herein for any embodiment or variation of a compound of formula (II). In some embodiments, n is 4. In some embodiments, each Y is independently halogen or —O(C₁-C₆ alkyl) optionally substituted by 1-5 groups independently selected from the group consisting of halogen, hydroxyl, —CN, and —NH₂. In some embodiments, each Y is independently F, Cl, or unsubstituted —O(C₁-C₃ alkyl). In some embodiments, each Y is independently F, Cl, or —OCH₃. In some embodiments, n is 4, two Y groups are F, and two Y groups are —OCH₃. In some embodiments, n is 4, two Y groups are Cl, and two Y groups are —OCH₃. In some embodiments, V is CH₂. In some embodiments, W is CH₂. In some embodiments, R¹ is H. In some embodiments, R⁵ is H. In some embodiments, R⁶ is H.

In some embodiments, the compound provided is of formula (II-b):

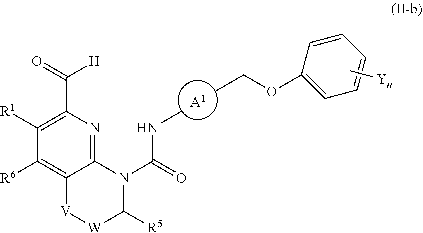

or a pharmaceutically acceptable salt thereof,
wherein Y, n, V, W, R¹, R⁵, and R⁶ are as defined herein for any embodiment or variation of a compound of formula (I), and the Ring A¹ moiety is as defined herein for any embodiment or variation of a compound of formula (II). In some embodiments, n is 4. In some embodiments, each Y is independently halogen or —O(C₁-C₆ alkyl) optionally substituted by 1-5 groups independently selected from the group consisting of halogen, hydroxyl, —CN, and —NH₂. In some embodiments, each Y is independently F, Cl, or unsubstituted —O(C₁-C₃ alkyl). In some embodiments, each Y is independently F, Cl, or —OCH₃. In some embodiments, n is 4, two Y groups are F, and two Y groups are —OCH₃. In some embodiments, n is 4, two Y groups are Cl, and two Y groups are —OCH₃. In some embodiments, V is CH₂. In some embodiments, W is CH₂. In some embodiments, R¹ is H. In some embodiments, R⁵ is H. In some embodiments, R⁶ is H.

In some embodiments, the compound provided is of formula (II-c):

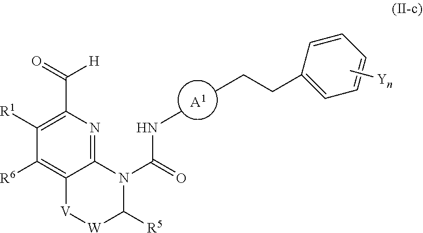

or a pharmaceutically acceptable salt thereof,
wherein Y, n, V, W, R¹, R⁵, and R⁶ are as defined herein for any embodiment or variation of a compound of formula (I), and the Ring A¹ moiety is as defined herein for any embodiment or variation of a compound of formula (II). In some embodiments, n is 4. In some embodiments, each Y is independently halogen or —O(C₁-C₆ alkyl) optionally substituted by 1-5 groups independently selected from the group consisting of halogen, hydroxyl, —CN, and —NH₂. In some embodiments, each Y is independently F, Cl, or unsubstituted —O(C₁-C₃ alkyl). In some embodiments, each Y is independently F, Cl, or —OCH₃. In some embodiments, n is 4, two Y groups are F, and two Y groups are —OCH₃. In some embodiments, n is 4, two Y groups are Cl, and two Y groups are —OCH₃. In some embodiments, V is CH₂. In some embodiments, W is CH₂. In some embodiments, R¹ is H. In some embodiments, R⁵ is H. In some embodiments, R⁶ is H.

In some embodiments, the compound provided is of formula (II-d):

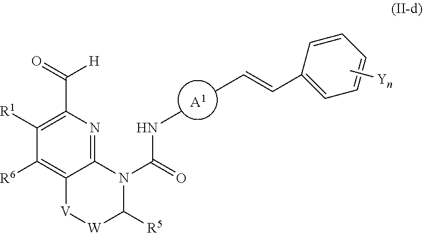

or a pharmaceutically acceptable salt thereof,
wherein Y, n, V, W, R¹, R⁵, and R⁶ are as defined herein for any embodiment or variation of a compound of formula (I), and the Ring A¹ moiety is as defined herein for any embodiment or variation of a compound of formula (II). In some embodiments, n is 4. In some embodiments, each Y is independently halogen or —O(C₁-C₆ alkyl) optionally substituted by 1-5 groups independently selected from the group consisting of halogen, hydroxyl, —CN, and —NH₂. In some embodiments, each Y is independently F, Cl, or unsubstituted —O(C₁-C₃ alkyl). In some embodiments, each Y is independently F, Cl, or —OCH₃. In some embodiments, n is 4, two Y groups are F, and two Y groups are —OCH₃. In some embodiments, n is 4, two Y groups are Cl, and two Y groups are —OCH₃. In some embodiments, V is CH₂. In some embodiments, W is CH₂. In some embodiments, R¹ is H. In some embodiments, R⁵ is H. In some embodiments, R⁶ is H.

In some embodiments, the compound provided is of formula (II-e):

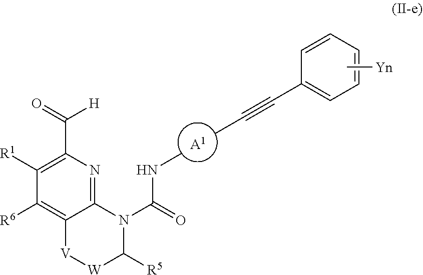

or a pharmaceutically acceptable salt thereof,
wherein Y, n, V, W, R¹, R⁵, and R⁶ are as defined herein for any embodiment or variation of a compound of formula (I), and the Ring A¹ moiety is as defined herein for any embodiment or variation of a compound of formula (II). In some embodiments, n is 4. In some embodiments, each Y is independently halogen or —O(C₁-C₆ alkyl) optionally substituted by 1-5 groups independently selected from the group consisting of halogen, hydroxyl, —CN, and —NH₂. In some embodiments, each Y is independently F, Cl, or unsubstituted —O(C₁-C₃ alkyl). In some embodiments, each Y is independently F, Cl, or —OCH₃. In some embodiments, n is 4, two Y groups are F, and two Y groups are —OCH₃. In some embodiments, n is 4, two Y groups are Cl, and two Y groups are —OCH₃. In some embodiments, V is CH₂. In some embodiments, W is CH₂. In some embodiments, R¹ is H. In some embodiments, R⁵ is H. In some embodiments, R⁶ is H.

In some embodiments, the compound provided is of formula (III):

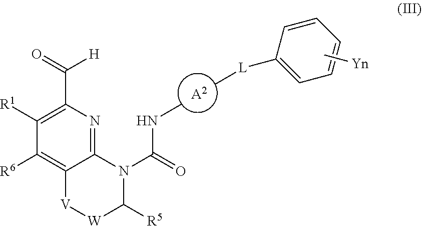

or a pharmaceutically acceptable salt thereof,
wherein L, Y, n, V, W, R¹, R⁵, and R⁶ are as defined herein for any embodiment or variation of a compound of formula (I), and

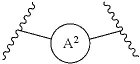

(i.e., the Ring A² moiety) is a 5-membered heteroarylene optionally substituted by 1-4 halogen or C₁-C₆ alkyl groups, provided that at least one Y, when present, is halogen. In some embodiments, the Ring A² moiety is pyrazolylene optionally substituted by 1-2 halogen or C₁-C₃ alkyl groups. In some embodiments, the Ring A² moiety is unsubstituted pyrazolylene, such as unsubstituted 3,5-pyrazolylene. In some embodiments, n is 4. In some embodiments, each Y is independently halogen or —O(C₁-C₆ alkyl) optionally substituted by 1-5 groups independently selected from the group consisting of halogen, hydroxyl, —CN, and —NH₂. In some embodiments, each Y is independently F, Cl, or unsubstituted —O(C₁-C₃ alkyl). In some embodiments, each Y is independently F, Cl, or —OCH₃. In some embodiments, n is 4, two Y groups are F, and two Y groups are —OCH₃. In some embodiments, n is 4, two Y groups are Cl, and two Y groups are —OCH₃. In some embodiments, V is CH₂. In some embodiments, W is CH₂. In some embodiments, R¹ is H. In some embodiments, R⁵ is H. In some embodiments, R⁶ is H.

In some embodiments, the compound provided is of formula (III-a):

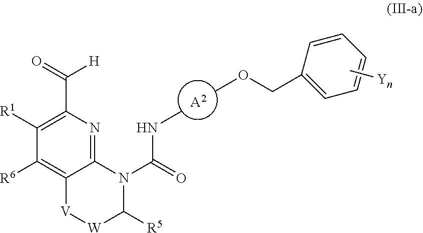

or a pharmaceutically acceptable salt thereof,
wherein Y, n, V, W, R¹, R⁵, and R⁶ are as defined herein for any embodiment or variation of a compound of formula (I), and the Ring A² moiety is as defined herein for any embodiment or variation of a compound of formula (III), provided that when n is 1-5, then at least one Y is halogen. In some embodiments, n is 4. In some embodiments, each Y is independently halogen or —O(C₁-C₆ alkyl) optionally substituted by 1-5 groups independently selected from the group consisting of halogen, hydroxyl, —CN, and —NH₂. In some embodiments, each Y is independently F, Cl, or unsubstituted —O(C₁-C₃ alkyl). In some embodiments, each Y is independently F, Cl, or —OCH₃. In some embodiments, n is 4, two Y groups are F, and two Y groups are —OCH₃. In some embodiments, n is 4, two Y groups are Cl, and two Y groups are —OCH₃. In some embodiments, V is CH₂. In some embodiments, W is CH₂. In some embodiments, R¹ is H. In some embodiments, R⁵ is H. In some embodiments, R⁶ is H.

In some embodiments, the compound provided is of formula (III-b):

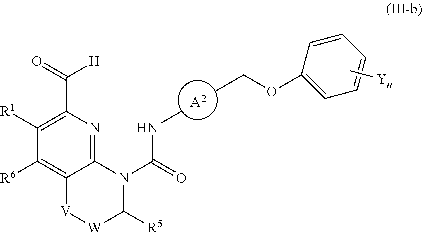

or a pharmaceutically acceptable salt thereof,
wherein Y, n, V, W, R¹, R⁵, and R⁶ are as defined herein for any embodiment or variation of a compound of formula (I), and the Ring A² moiety is as defined herein for any embodiment or variation of a compound of formula (III), provided that when n is 1-5, then at least one Y is halogen. In some embodiments, n is 4. In some embodiments, each Y is independently halogen or —O(C₁-C₆ alkyl) optionally substituted by 1-5 groups independently selected from the group consisting of halogen, hydroxyl, —CN, and —NH₂. In some embodiments, each Y is independently F, Cl, or unsubstituted —O(C₁-C₃ alkyl). In some embodiments, each Y is independently F, Cl, or —OCH₃. In some embodiments, n is 4, two Y groups are F, and two Y groups are —OCH₃. In some embodiments, n is 4, two Y groups are Cl, and two Y groups are —OCH₃. In some embodiments, V is CH₂. In some embodiments, W is CH₂. In some embodiments, R¹ is H. In some embodiments, R⁵ is H. In some embodiments, R⁶ is H.

In some embodiments, the compound provided is of formula (III-c):

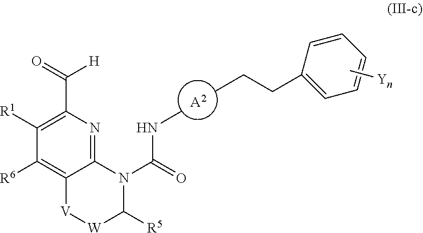

or a pharmaceutically acceptable salt thereof,
wherein Y, n, V, W, R¹, R⁵, and R⁶ are as defined herein for any embodiment or variation of a compound of formula (I), and the Ring A² moiety is as defined herein for any embodiment or variation of a compound of formula (III), provided that when n is 1-5, then at least one Y is halogen. In some embodiments, n is 4. In some embodiments, each Y is independently halogen or —O(C₁-C₆ alkyl) optionally substituted by 1-5 groups independently selected from the group consisting of halogen, hydroxyl, —CN, and —NH₂. In some embodiments, each Y is independently F, Cl, or unsubstituted —O(C₁-C₃ alkyl). In some embodiments, each Y is independently F, Cl, or —OCH₃. In some embodiments, n is 4, two Y groups are F, and two Y groups are —OCH₃. In some embodiments, n is 4, two Y groups are Cl, and two Y groups are —OCH₃. In some embodiments, V is CH₂. In some embodiments, W is CH₂. In some embodiments, R¹ is H. In some embodiments, R⁵ is H. In some embodiments, R⁶ is H.

In some embodiments, the compound provided is of formula (III-d):

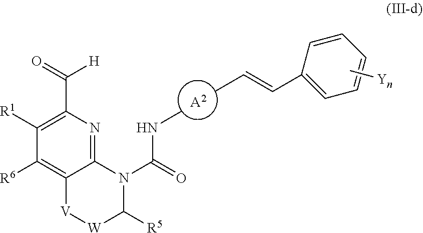

or a pharmaceutically acceptable salt thereof,
wherein Y, n, V, W, R¹, R⁵, and R⁶ are as defined herein for any embodiment or variation of a compound of formula (I), and the Ring A² moiety is as defined herein for any embodiment or variation of a compound of formula (III), provided that when n is 1-5, then at least one Y is halogen. In some embodiments, n is 4. In some embodiments, each Y is independently halogen or —O(C₁-C₆ alkyl) optionally substituted by 1-5 groups independently selected from the group consisting of halogen, hydroxyl, —CN, and —NH₂. In some embodiments, each Y is independently F, Cl, or unsubstituted —O(C₁-C₃ alkyl). In some embodiments, each Y is independently F, Cl, or —OCH₃. In some embodiments, n is 4, two Y groups are F, and two Y groups are —OCH₃. In some embodiments, n is 4, two Y groups are Cl, and two Y groups are —OCH₃. In some embodiments, V is CH₂. In some embodiments, W is CH₂. In some embodiments, R¹ is H. In some embodiments, R⁵ is H. In some embodiments, R⁶ is H.

In some embodiments, the compound provided is of formula (III-e):

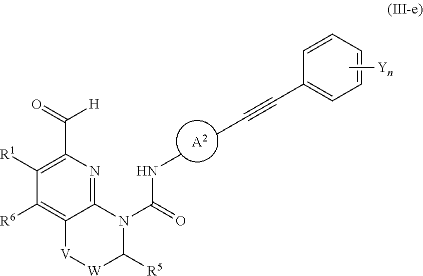

or a pharmaceutically acceptable salt thereof,
wherein Y, n, V, W, R¹, R⁵, and R⁶ are as defined herein for any embodiment or variation of a compound of formula (I), and the Ring A² moiety is as defined herein for any embodiment or variation of a compound of formula (III), provided that when n is 1-5, then at least one Y is halogen. In some embodiments, n is 4. In some embodiments, each Y is independently halogen or —O(C₁-C₆ alkyl) optionally substituted by 1-5 groups independently selected from the group consisting of halogen, hydroxyl, —CN, and —NH₂. In some embodiments, each Y is independently F, Cl, or unsubstituted —O(C₁-C₃ alkyl). In some embodiments, each Y is independently F, Cl, or —OCH₃. In some embodiments, n is 4, two Y groups are F, and two Y groups are —OCH₃. In some embodiments, n is 4, two Y groups are Cl, and two Y groups are —OCH₃. In some embodiments, V is CH₂. In some embodiments, W is CH₂. In some embodiments, R¹ is H. In some embodiments, R⁵ is H. In some embodiments, R⁶ is H.

In some embodiments, the compound provided is of formula (IV):

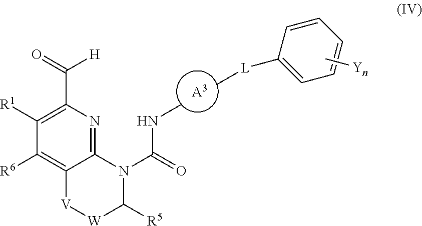

or a pharmaceutically acceptable salt thereof,
wherein L, Y, n, V, W, R¹, R⁵, and R⁶ are as defined herein for any embodiment or variation of a compound of formula (I); and

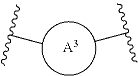

(i.e., the Ring A³ moiety) is a 6-membered heteroarylene optionally substituted by 1-4 halogen or C₁-C₆ alkyl groups. In some embodiments, the Ring A² moiety is pyridinylene optionally substituted by 1-2 halogen or C₁-C₃ alkyl groups. In some embodiments, the Ring A² moiety is unsubstituted pyridinylene, such as unsubstituted 2,5-pyridinylene. In some embodiments, the Ring A² moiety is pyrimidinylene optionally substituted by 1-2 halogen or C₁-C₃ alkyl groups. In some embodiments, the Ring A² moiety is unsubstituted pyrimidinylene, such as unsubstituted 2,5-pyrimidinylene. In some embodiments, n is 4. In some embodiments, each Y is independently halogen or —O(C₁-C₆ alkyl) optionally substituted by 1-5 groups independently selected from the group consisting of halogen, hydroxyl, —CN, and —NH₂. In some embodiments, each Y is independently F, Cl, or unsubstituted —O(C₁-C₃ alkyl). In some embodiments, each Y is independently F, Cl, or —OCH₃. In some embodiments, n is 4, two Y groups are F, and two Y groups are —OCH₃. In some embodiments, n is 4, two Y groups are Cl, and two Y groups are —OCH₃. In some embodiments, V is CH₂. In some embodiments, W is CH₂. In some embodiments, R¹ is H. In some embodiments, R⁵ is H. In some embodiments, R⁶ is H or R⁶ is C₁-C₆ alkyl. In some embodiments, R⁶ is H. In some embodiments, R⁶ is C₁-C₃ alkyl. In some embodiments, R⁶ is —CH₃.

In some embodiments, the compound provided is of formula (IV-a):

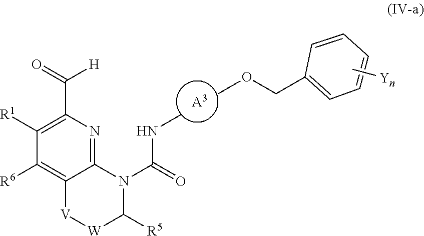

or a pharmaceutically acceptable salt thereof,
wherein Y, n, V, W, R¹, R⁵, and R⁶ are as defined herein for any embodiment or variation of a compound of formula (I), and the Ring A³ moiety is as defined herein for any embodiment or variation of a compound of formula (IV). In some embodiments, n is 4. In some embodiments, each Y is independently halogen or —O(C₁-C₆ alkyl) optionally substituted by 1-5 groups independently selected from the group consisting of halogen, hydroxyl, —CN, and —NH₂. In some embodiments, each Y is independently F, Cl, or unsubstituted —O(C₁-C₃ alkyl). In some embodiments, each Y is independently F, Cl, or —OCH₃. In some embodiments, n is 4, two Y groups are F, and two Y groups are —OCH₃. In some embodiments, n is 4, two Y groups are Cl, and two Y groups are —OCH₃. In some embodiments, V is CH₂. In some embodiments, W is CH₂. In some embodiments, R¹ is H. In some embodiments, R⁵ is H. In some embodiments, R⁶ is H or R⁶ is C₁-C₆ alkyl. In some embodiments, R⁶ is H. In some embodiments, R⁶ is C₁-C₃ alkyl. In some embodiments, R⁶ is —CH₃.

In some embodiments, the compound provided is of formula (IV-b):

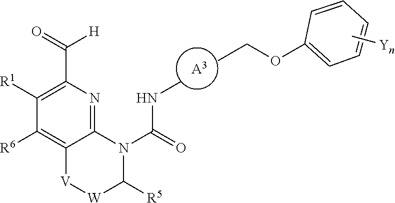

or a pharmaceutically acceptable salt thereof,
wherein Y, n, V, W, R¹, R⁵, and R⁶ are as defined herein for any embodiment or variation of a compound of formula (I), and the Ring A³ moiety is as defined herein for any embodiment or variation of a compound of formula (IV). In some embodiments, n is 4. In some embodiments, each Y is independently halogen or —O(C₁-C₆ alkyl) optionally substituted by 1-5 groups independently selected from the group consisting of halogen, hydroxyl, —CN, and —NH₂. In some embodiments, each Y is independently F, Cl, or unsubstituted —O(C₁-C₃ alkyl). In some embodiments, each Y is independently F, Cl, or —OCH₃. In some embodiments, n is 4, two Y groups are F, and two Y groups are —OCH₃. In some embodiments, n is 4, two Y groups are Cl, and two Y groups are —OCH₃. In some embodiments, V is CH₂. In some embodiments, W is CH₂. In some embodiments, R¹ is H. In some embodiments, R⁵ is H. In some embodiments, R⁶ is H.

In some embodiments, the compound provided is of formula (IV-c):

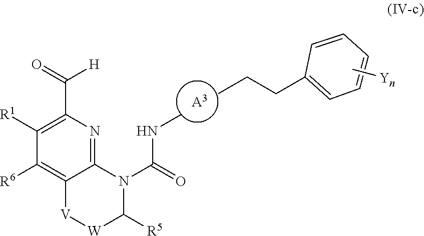

or a pharmaceutically acceptable salt thereof,
wherein Y, n, V, W, R¹, R⁵, and R⁶ are as defined herein for any embodiment or variation of a compound of formula (I), and the Ring A³ moiety is as defined herein for any embodiment or variation of a compound of formula (IV). In some embodiments, n is 4. In some embodiments, each Y is independently halogen or —O(C₁-C₆ alkyl) optionally substituted by 1-5 groups independently selected from the group consisting of halogen, hydroxyl, —CN, and —NH₂. In some embodiments, each Y is independently F, Cl, or unsubstituted —O(C₁-C₃ alkyl). In some embodiments, each Y is independently F, Cl, or —OCH₃. In some embodiments, n is 4, two Y groups are F, and two Y groups are —OCH₃. In some embodiments, n is 4, two Y groups are Cl, and two Y groups are —OCH₃. In some embodiments, V is CH₂. In some embodiments, W is CH₂. In some embodiments, R¹ is H. In some embodiments, R⁵ is H. In some embodiments, R⁶ is H.

In some embodiments, the compound provided is of formula (IV-d):

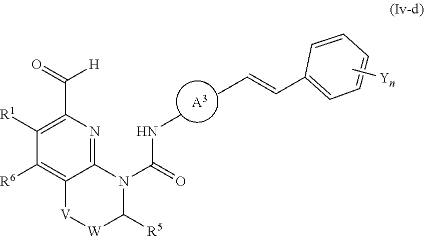

or a pharmaceutically acceptable salt thereof,
wherein Y, n, V, W, R¹, R⁵, and R⁶ are as defined herein for any embodiment or variation of a compound of formula (I), and the Ring A³ moiety is as defined herein for any embodiment or variation of a compound of formula (IV). In some embodiments, n is 4. In some embodiments, each Y is independently halogen or —O(C₁-C₆ alkyl) optionally substituted by 1-5 groups independently selected from the group consisting of halogen, hydroxyl, —CN, and —NH₂. In some embodiments, each Y is independently F, Cl, or unsubstituted —O(C₁-C₃ alkyl). In some embodiments, each Y is independently F, Cl, or —OCH₃. In some embodiments, n is 4, two Y groups are F, and two Y groups are —OCH₃. In some embodiments, n is 4, two Y groups are Cl, and two Y groups are —OCH₃. In some embodiments, V is CH₂. In some embodiments, W is CH₂. In some embodiments, R¹ is H. In some embodiments, R⁵ is H. In some embodiments, R⁶ is H.

In some embodiments, the compound provided is of formula (IV-e):

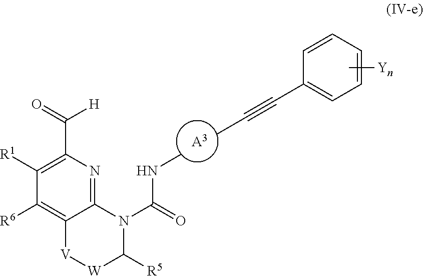

or a pharmaceutically acceptable salt thereof,
wherein Y, n, V, W, R¹, R⁵, and R⁶ are as defined herein for any embodiment or variation of a compound of formula (I), and the Ring A³ moiety is as defined herein for any embodiment or variation of a compound of formula (IV). In some embodiments, n is 4. In some embodiments, each Y is independently halogen or —O(C₁-C₆ alkyl) optionally substituted by 1-5 groups independently selected from the group consisting of halogen, hydroxyl, —CN, and —NH₂. In some embodiments, each Y is independently F, Cl, or unsubstituted —O(C₁-C₃ alkyl). In some embodiments, each Y is independently F, Cl, or —OCH₃. In some embodiments, n is 4, two Y groups are F, and two Y groups are —OCH₃. In some embodiments, n is 4, two Y groups are Cl, and two Y groups are —OCH₃. In some embodiments, V is CH₂. In some embodiments, W is CH₂. In some embodiments, R¹ is H. In some embodiments, R⁵ is H. In some embodiments, R⁶ is H.

In some embodiments, the compound provided is of formula (V):

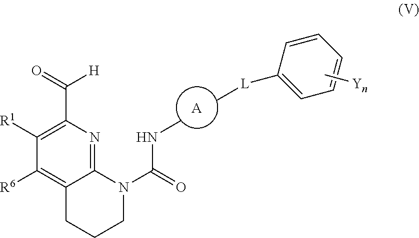

or a pharmaceutically acceptable salt thereof,
wherein the Ring A moiety, L, Y, n, R¹, and R⁶ are as defined herein for any embodiment or variation of a compound of formula (I). In some embodiments, the Ring A moiety is 5- to 6-membered heteroarylene optionally substituted by 1-4 halogen or C₁-C₆ alkyl groups. In some embodiments, the Ring A moiety is pyrazolylene, pyridinylene, or pyrimidinylene optionally substituted by 1-2 halogen or C₁-C₃ alkyl groups. In some embodiments, the Ring A moiety is

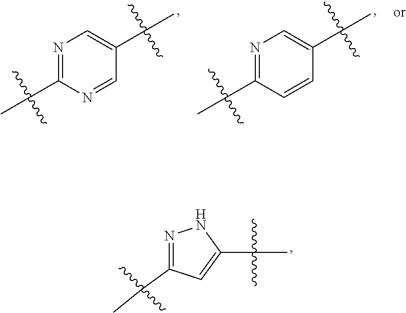 [SMILES: *c1cnc(*)nc1 ; *c1ccc(*)nc1 ; *c1cc(*)[nH]n1]

each of which is unsubstituted. In some embodiments, L is —OCH₂—, —CH₂CH₂—,

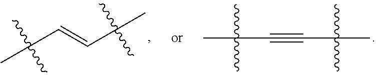

In some embodiments, n is 4. In some embodiments, each Y is independently halogen or —O(C₁-C₆ alkyl) optionally substituted by 1-5 groups independently selected from the group consisting of halogen, hydroxyl, —CN, and
—NH₂. In some embodiments, each Y is independently F, Cl, or unsubstituted —O(C₁-C₃ alkyl). In some embodiments, each Y is independently F, Cl, or —OCH₃. In some embodiments, n is 4, two Y groups are F, and two Y groups are —OCH₃. In some embodiments, n is 4, two Y groups are Cl, and two Y groups are —OCH₃. In some embodiments, R¹ is H or -CH₂NR²R³, wherein R² and R³ are taken together with the nitrogen atom to which they are attached to form a 5- to 6-membered heterocyclyl optionally containing one additional heteroatom or heteroatom-containing moiety selected from the group consisting of N, N-oxide, O, and S, wherein the heterocyclyl is optionally substituted by 1-5 R⁴ groups. In some embodiments, R¹ is H. In some embodiments, R¹ is CH₂NR²R³, wherein R² and R³ are taken together with the nitrogen atom to which they are attached to form a 6-membered heterocyclyl optionally containing one additional nitrogen atom, wherein the heterocyclyl is optionally substituted by 1-5 R⁴ groups. In some embodiments, R¹ is CH₂NR²R³, wherein R² and R³ are taken together with the nitrogen atom to which they are attached to form piperazinyl optionally substituted by 1-5 R⁴ groups. In some embodiments, R¹ is CH₂NR²R³, wherein R² and R³ are taken together with the nitrogen atom to which they are attached to form

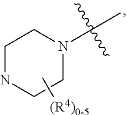

wherein the nitrogen at the 4-position of the ring is bound to H when not substituted by R⁴. In some embodiments, R⁴ is C₁-C₆ alkyl. In some embodiments, R⁴ is C₁-C₃ alkyl. In some embodiments, R⁴ is —CH₃. In some embodiments, two R⁴ groups attached to the same ring atom are taken together to form an oxo group. In some embodiments, two R⁴ groups attached to the same ring atom are taken together to form an oxo group and one additional R⁴ group is —CH₃. In some embodiments, R⁶ is H or R⁶ is C₁-C₆ alkyl. In some embodiments, R⁶ is H. In some embodiments, R⁶ is C₁-C₃ alkyl. In some embodiments, R⁶ is
—CH₃.

In some embodiments, the compound provided is of formula (VI):

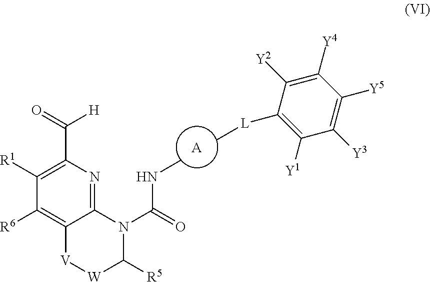

or a pharmaceutically acceptable salt thereof,
wherein the Ring A moiety, L, V, W, R¹, R⁵, and R⁶ are as defined herein for any embodiment or variation of a compound of formula (I); Y¹ and Y² are independently hydrogen or halogen; and Y³, Y⁴, and Y⁵ are independently hydrogen, halogen, or —O(C₁-C₆ alkyl) optionally substituted by 1-5 groups independently selected from the group consisting of halogen, hydroxyl, —CN, and —NH₂, provided that when the Ring A moiety is 5-membered heteroarylene, then (i) at least one of Y¹, Y², Y³, Y⁴, or Y⁵ is halogen, or (ii) Y¹, Y², Y³, Y⁴, and Y⁵ are each hydrogen. In some embodiments, Y¹ and Y² are independently halogen; Y³ and Y⁴ are independently —O(C₁-C₃ alkyl) optionally substituted by 1-5 groups independently selected from the group consisting of halogen, hydroxyl, —CN, and —NH₂; and Y⁵ is hydrogen. In some embodiments, Y¹ and Y² are independently F or Cl; Y³ and Y⁴ are independently unsubstituted —O(C₁-C₃ alkyl); and Y⁵ is hydrogen. In some embodiments, Y¹ and Y² are each F; Y³ and Y⁴ are each —OCH_{3;} and Y⁵ is hydrogen. In some embodiments, Y¹ and Y² are each Cl; Y³ and Y⁴ are each —OCH_{3;} and Y⁵ is hydrogen. In some embodiments, the Ring A moiety is 5- to 6-membered heteroarylene optionally substituted by 1-4 halogen or C₁-C₆ alkyl groups. In some embodiments, the Ring A moiety is pyrazolylene, pyridinylene, or pyrimidinylene optionally substituted by 1-2 halogen or C₁-C₃ alkyl groups. In some embodiments, the Ring A moiety is

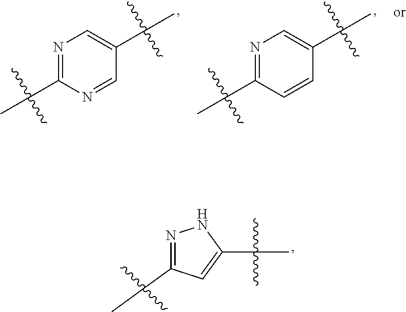 [SMILES: *c1cnc(*)nc1 ; *c1ccc(*)nc1 ; *c1cc(*)[nH]n1]

each of which is unsubstituted. In some embodiments, L is —OCH₂—, —CH₂CH₂—,

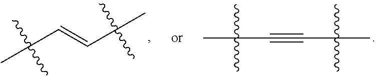

In some embodiments, V is CH₂. In some embodiments, W is CH₂. In some embodiments, R¹ is H or —CH₂NR²R³, wherein R² and R³ are taken together with the nitrogen atom to which they are attached to form a 5- to 6-membered heterocyclyl optionally containing one additional heteroatom or heteroatom-containing moiety selected from the group consisting of N, N-oxide, O, and S, wherein the heterocyclyl is optionally substituted by 1-5 R⁴ groups. In some embodiments, R¹ is H. In some embodiments, R¹ is CH₂NR²R³, wherein R² and R³ are taken together with the nitrogen atom to which they are attached to form a 6-membered heterocyclyl optionally containing one additional nitrogen atom, wherein the heterocyclyl is optionally substituted by 1-5 R⁴ groups. In some embodiments, R¹ is CH₂NR²R³, wherein R² and R³ are taken together with the nitrogen atom to which they are attached to form piperazinyl optionally substituted by 1-5 R⁴ groups. In some embodiments, R¹ is CH₂NR²R³, wherein R² and R³ are taken together with the nitrogen atom to which they are attached to form

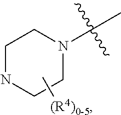

wherein the nitrogen atom at the 4-position of the ring is bound to H when not substituted by R⁴. In some embodiments, R⁴ is C₁-C₆ alkyl. In some embodiments, R⁴ is C₁-C₃ alkyl. In some embodiments, R⁴ is —CH₃. In some embodiments, two R⁴ groups attached to the same ring atom are taken together to form an oxo group. In some embodiments, two R⁴ groups attached to the same ring atom are taken together to form an oxo group and one additional R⁴ group is —CH₃. In some embodiments, R⁵ is H. In some embodiments, R⁶ is H or R⁶ is C₁-C₆ alkyl. In some embodiments, R⁶ is H. In some embodiments, R⁶ is C₁-C₃ alkyl. In some embodiments, R⁶ is —CH₃.

In some embodiments, the compound provided is of formula (VI-A):

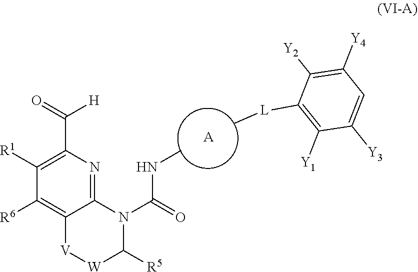

or a pharmaceutically acceptable salt thereof,
wherein the Ring A moiety, L, V, W, R¹, R⁵, and R⁶ are as defined herein for any embodiment or variation of a compound of formula (I); Y¹ and Y² are independently hydrogen or halogen; and Y³ and Y⁴ are independently hydrogen, halogen, or —O(C₁-C₆ alkyl) optionally substituted by 1-5 groups independently selected from the group consisting of halogen, hydroxyl, —CN, and —NH₂, provided that when the Ring A moiety is 5-membered heteroarylene, then (i) at least one of Y¹, Y², Y³, or Y⁴ is halogen or (ii) Y¹, Y², Y³, and Y⁴ are each hydrogen. In some embodiments, Y¹ and Y² are independently halogen, and Y³ and Y⁴ are independently —O(C₁-C₃ alkyl) optionally substituted by 1-5 groups independently selected from the group consisting of halogen, hydroxyl, —CN, and —NH₂. In some embodiments, Y¹ and Y² are independently F or Cl, and Y³ and Y⁴ are independently unsubstituted —O(C₁-C₃ alkyl). In some embodiments, Y¹ and Y² are each F, and Y³ and Y⁴ are each —OCH₃. In some embodiments, the Ring A moiety is 5- to 6-membered heteroarylene optionally substituted by 1-4 halogen or C₁-C₆ alkyl groups. In some embodiments, the Ring A moiety is pyrazolylene, pyridinylene, or pyrimidinylene optionally substituted by 1-2 halogen or C₁-C₃ alkyl groups. In some embodiments, the Ring A moiety is

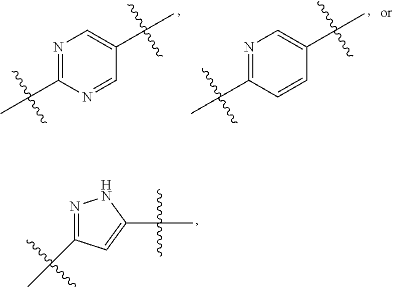 [SMILES: *c1cnc(*)nc1 ; *c1ccc(*)nc1 ; *c1cc(*)[nH]n1]

each of which is unsubstituted. In some embodiments, L is —OCH₂—, —CH₂CH₂—,

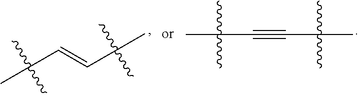

In some embodiments, V is CH₂. In some embodiments, W is CH₂. In some embodiments, R¹ is H or —CH₂NR²R³, wherein R² and R³ are taken together with the nitrogen atom to which they are attached to form a 5- to 6-membered heterocyclyl optionally containing one additional heteroatom or heteroatom-containing moiety selected from the group consisting of N, N-oxide, O, and S, wherein the heterocyclyl is optionally substituted by 1-5 R⁴ groups. In some embodiments, R¹ is H. In some embodiments, R¹ is CH₂NR²R³, wherein R² and R³ are taken together with the nitrogen atom to which they are attached to form a 6-membered heterocyclyl optionally containing one additional nitrogen atom, wherein the heterocyclyl is optionally substituted by 1-5 R⁴ groups. In some embodiments, R¹ is CH₂NR²R³, wherein R² and R³ are taken together with the nitrogen atom to which they are attached to form piperazinyl optionally substituted by 1-5 R⁴ groups. In some embodiments, R¹ is CH₂NR²R³, wherein R² and R³ are taken together with the nitrogen atom to which they are attached to form

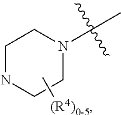

wherein the nitrogen atom at the 4-position of the ring is bound to H when not substituted by R⁴. In some embodiments, R⁴ is C₁-C₆ alkyl. In some embodiments, R⁴ is C₁-C₃ alkyl. In some embodiments, R⁴ is —CH₃. In some embodiments, two R⁴ groups attached to the same ring atom are taken together to form an oxo group. In some embodiments, two R⁴ groups attached to the same ring atom are taken together to form an oxo group and one additional R⁴ group is —CH₃. In some embodiments, R⁵ is H. In some embodiments, R⁶ is H or R⁶ is C₁-C₆ alkyl. In some embodiments, R⁶ is H. In some embodiments, R⁶ is C₁-C₃ alkyl. In some embodiments, R⁶ is —CH₃.

In one aspect, provided is a compound of formula (I), or a pharmaceutically acceptable salt thereof, which has any one or more of the following structural features:

(I) the Ring A moiety is:
(i) 5-membered heteroarylene optionally substituted by 1-4 halogen or C₁-C₆ alkyl groups; or
(ii) 6-membered heteroarylene optionally substituted by 1-4 halogen or C₁-C₆ alkyl groups;

(II) L is:
(iii) —OCH₂—;
(iv) —CH₂CH₂—;

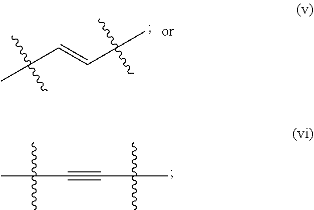

(III) V is CH₂;

(IV) W is CH₂;

(V) n is 4;

(VI) each Y is independently:
(vii) halogen; or
(viii) —O(C₁-C₆ alkyl) optionally substituted by 1-2 groups independently selected from the group consisting of halogen, hydroxyl, —CN, and —NH₂,

(VII) R¹ is:
(ix) H; or
(x) —CH₂NR²R³, wherein R² and R³ are taken together with the nitrogen atom to which they are attached to form a 5- to 6-membered heterocyclyl optionally containing one additional heteroatom selected from the group consisting of N and O, wherein the heterocyclyl is optionally substituted by 1-5 R⁴ groups;

(VIII) each R⁴ is independently:
(xi) C₁-C₆ alkyl; or
(xii) taken together with another R⁴ groups attached to the same ring atom to form an oxo group;

(IX) R⁵ is H; and

(X) R⁶ is:
(xiii) H; or
(xiv) C₁-C₆ alkyl;
   provided that when (I)(i) applies, then (VI)(vii) applies. In one variation, (I) applies. In one variation, (II) applies. In one variation, (III) applies. In one variation, (IV) applies. In one variation, (V) applies. In one variation, (VI) applies. In one variation, (VII) applies. In one variation, (I), (II), (III), (VI), (V), (VI), (VII), (VII), (IX), and (X) apply. In one variation, (I), (II), (III), (VI), (V), (VI), (VII), (IX), and (X) apply. In one variation, (i), (iii), (III), (IV), (V), (vii), (viii), (ix), (IX), and (xiii) apply. In one variation, (ii), (iii), (III), (IV), (V), (vii), (viii), (ix), (IX), and (xiii) apply. In one variation, (ii), (iii), (III), (IV), (V), (vii), (viii), (x), (xi), (xii), (IX), and (xiii) apply. In one variation, (ii), (iii), (III), (IV), (V), (vii), (viii), (ix), (IX), and (xiv) apply. In one variation, (ii), (iv), (III), (IV), (V), (vii), (viii), (ix), (IX), and (xiii) apply. In one variation, (ii), (v), (III), (IV), (V), (vii), (viii), (ix), (IX), and (xiii) apply. In one variation, (ii), (vi), (III), (IV), (V), (vii), (viii), (ix), (IX), and (xiii) apply.

In the descriptions herein, it is understood that every description, variation, embodiment or aspect of a moiety may be combined with every description, variation, embodiment or aspect of other moieties the same as if each and every combination of descriptions is specifically and individually listed. For example, every description, variation, embodiment or aspect provided herein with respect to the Ring A moiety of formula (I) may be combined with every description, variation, embodiment or aspect of L, Y, n, V, W, R¹, R², R³, R⁴, R⁵, and R⁶ the same as if each and every combination were specifically and individually listed. It is also understood that all descriptions, variations, embodiments or aspects of formula (I), where applicable, apply equally to other formulae detailed herein, and are equally described, the same as if each and every description, variation, embodiment or aspect were separately and individually listed for all formulae. For example, all descriptions, variations, embodiments or aspects of formula (I), where applicable, apply equally to any of formulae as detailed herein, such as formulae (I-a), (I-b), (I-c),(I-d), (I-e), (II), (II-a), (II-b), (II-c), (II-d), (II-e), (III), (III-a), (III-b), (III-c), (III-d), (III-e), (IV), (IV-a), (IV-b), (IV-c),(IV-d), (IV-e), (V), (VI), and (VI-a), and are equally described, the same as if each and every description, variation, embodiment or aspect were separately and individually listed for all formulae.

In some embodiments, provided is a compound selected from the compounds in Table 1, or pharmaceutically acceptable salt thereof. Although certain compounds described in Table 1 are presented as specific stereoisomers and/or in a non-stereochemical form, it is understood that any or all stereochemical forms, including any enantiomeric or diastereomeric forms, and any tautomers or other forms of any of the compounds of Table 1 are herein described.

TABLE 1

| Example | Structure |
|---|---|
| 1 | 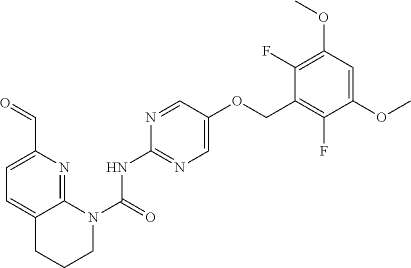 [SMILES: COc1cc(OC)c(F)c(COc2cnc(NC(=O)N3CCCc4ccc(C=O)nc43)nc2)c1F] |
| 2 | 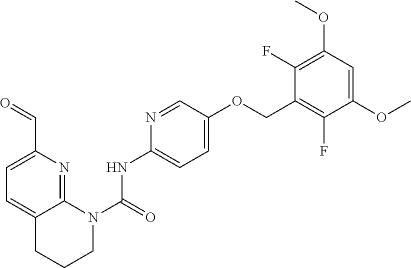 [SMILES: COc1cc(OC)c(F)c(COc2ccc(NC(=O)N3CCCc4ccc(C=O)nc43)nc2)c1F] |
| 3 | 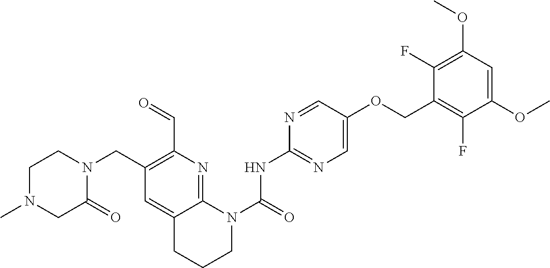 |
| 4 | 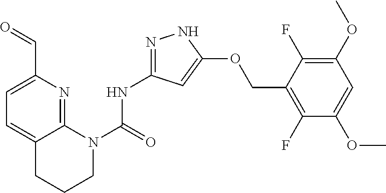 [SMILES: COc1cc(OC)c(F)c(COc2cc(NC(=O)N3CCCc4ccc(C=O)nc43)n[nH]2)c1F] |
| 5 | 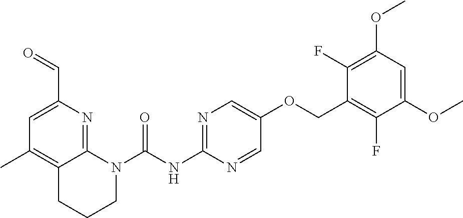 |
| 6 | 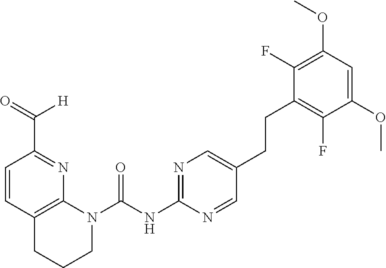 [SMILES: COc1cc(OC)c(F)c(CCc2cnc(NC(=O)N3CCCc4ccc(C=O)nc43)nc2)c1F] |
| 7 | 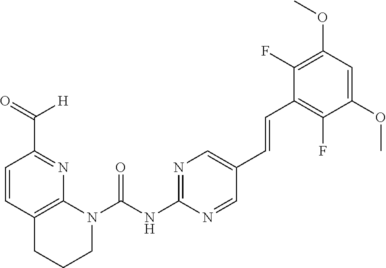 [SMILES: COc1cc(OC)c(F)c(/C=C/c2cnc(NC(=O)N3CCCc4ccc(C=O)nc43)nc2)c1F] |
| 8 | 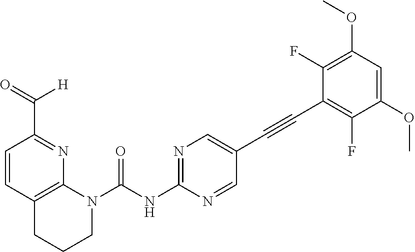 [SMILES: COc1cc(OC)c(F)c(C#Cc2cnc(NC(=O)N3CCCc4ccc(C=O)nc43)nc2)c1F] |

Also provided are salts of compounds referred to herein, such as pharmaceutically acceptable salts. The present disclosure also includes any or all of the stereochemical forms, including any enantiomeric or diastereomeric forms, and any tautomers or other forms of the compounds described. Thus, if a particular stereochemical form, such as a specific enantiomeric form or diastereomeric form, is depicted for a given compound, then it is understood that any or all stereochemical forms, including any enantiomeric or diastereomeric forms, and any tautomers or other forms of any of that same compound are herein described. Where tautomeric forms may be present for any of the compounds described herein, each and every tautomeric form is intended even though only one or some of the tautomeric forms may be explicitly depicted. The tautomeric forms specifically depicted may or may not be the predominant forms in solution or when used according to the methods described herein.

The disclosure also intends isotopically-labeled and/or isotopically-enriched forms of compounds described herein. The compounds herein may contain unnatural proportions of atomic isotopes at one or more of the atoms that constitute such compounds. In some embodiments, the compound is isotopically-labeled, such as an isotopically-labeled compound of the formula (I) or variations thereof described herein, where a fraction of one or more atoms are replaced by an isotope of the same element. Exemplary isotopes that can be incorporated into compounds described herein include isotopes of hydrogen, carbon, nitrogen, oxygen, phosphorus, sulfur, chlorine, such as ²H, ³H, ¹¹C, ¹³C, ¹⁴C ¹³N, ¹⁵O, ¹⁷O, ³²P, ³⁵S, ¹⁸F, ³⁶Cl. Certain isotope labeled compounds (e.g. ³H and ¹⁴C) are useful in compound or substrate tissue distribution studies. Incorporation of heavier isotopes such as deuterium (²H) can afford certain therapeutic advantages resulting from greater metabolic stability, for example, increased in vivo half-life, or reduced dosage requirements and, hence may be preferred in some instances. Isotopically-labeled compounds described herein can generally be prepared by standard methods and techniques known to those skilled in the art or by procedures similar to those described in the accompanying Examples substituting appropriate isotopically-labeled reagents in place of the corresponding non-labeled reagent.

Solvates of a compound provided herein or a salt thereof are also contemplated. Solvates contain either stoichiometric or non-stoichiometric amounts of a solvent, and are often formed during the process of crystallization. Hydrates are formed when the solvent is water, or alcoholates are formed when the solvent is alcohol.

A compound as detailed herein may in one aspect be in a purified form and compositions comprising a compound in purified forms are detailed herein. Compositions comprising a compound as detailed herein or a salt thereof are provided, such as compositions of substantially pure compounds. In some embodiments, a composition containing a compound as detailed herein or a salt thereof is in substantially pure form. Unless otherwise stated, “substantially pure” intends a composition that contains no more than 35% impurity, wherein the impurity denotes a compound other than the compound comprising the majority of the composition or a salt thereof. In some embodiments, a composition of substantially pure compound or a salt thereof is provided wherein the composition contains no more than 25%, 20%, 15%, 10%, or 5% impurity. In some embodiments, a composition of substantially pure compound or a salt thereof is provided wherein the composition contains or no more than 3%, 2%, 1% or 0.5% impurity.

Articles of manufacture comprising a compound described herein, or a salt or solvate thereof, in a suitable container are provided. The container may be a vial, jar, ampoule, preloaded syringe, i.v. bag, and the like.

In some embodiments, the compounds detailed herein are orally bioavailable. In some embodiments, the compounds detailed herein are formulated for parenteral (e.g., intravenous) administration.

One or several compounds described herein can be used in the preparation of a medicament by combining the compound or compounds disclosed herein with a pharmacologically acceptable carrier, which are known in the art. Depending on the therapeutic form of the medication, the carrier may be in various forms. In one variation, the manufacture of a medicament is for use in any of the methods disclosed herein, e.g., for the treatment of liver cancer.

### Pharmaceutical Compositions and Formulations

Any of the compounds described herein may be formulated as a pharmaceutically acceptable composition.

Pharmaceutical compositions of any of the compounds detailed herein are embraced by this disclosure. Thus, the present disclosure includes pharmaceutical compositions comprising a compound as detailed herein, or a pharmaceutically acceptable salt thereof, and a pharmaceutically acceptable carrier or excipient. In one aspect, the pharmaceutically acceptable salt is an acid addition salt, such as a salt formed with an inorganic or organic acid. Pharmaceutical compositions may take a form suitable for oral, buccal, parenteral, nasal, topical or rectal administration or a form suitable for administration by inhalation.

A compound as detailed herein may in one aspect be in a purified form and compositions comprising a compound in purified forms are detailed herein. Compositions comprising a compound as detailed herein or a salt thereof are provided, such as compositions of substantially pure compounds. In some embodiments, a composition containing a compound as detailed herein or a salt thereof is in substantially pure form.

In one variation, the compounds herein are synthetic compounds prepared for administration to an individual. In another variation, compositions are provided containing a compound in substantially pure form. In another variation, the present disclosure embraces pharmaceutical compositions comprising a compound detailed herein and a pharmaceutically acceptable carrier. In another variation, methods of administering a compound are provided. The purified forms, pharmaceutical compositions and methods of administering the compounds are suitable for any compound or form thereof detailed herein.

A compound detailed herein, or a pharmaceutically acceptable salt thereof, may be formulated for any available delivery route, including an oral, mucosal (e.g., nasal, sublingual, vaginal, buccal or rectal), parenteral (e.g., intramuscular, subcutaneous or intravenous), topical or transdermal delivery form. A compound or salt thereof may be formulated with suitable carriers to provide delivery forms that include, but are not limited to, tablets, caplets, capsules (such as hard gelatin capsules or soft elastic gelatin capsules), cachets, troches, lozenges, gums, dispersions, suppositories, ointments, cataplasms (poultices), pastes, powders, dressings, creams, solutions, patches, aerosols (e.g., nasal spray or inhalers), gels, suspensions (e.g., aqueous or non-aqueous liquid suspensions, oil-in-water emulsions or water-in-oil liquid emulsions), solutions and elixirs.

A compound detailed herein, or a pharmaceutically acceptable salt thereof, can be used in the preparation of a formulation, such as a pharmaceutical formulation, by combining the compound or compounds, or a salt thereof, with a pharmaceutically acceptable carrier. Depending on the therapeutic form of the system (e.g., transdermal patch vs. oral tablet), the carrier may be in various forms. In addition, pharmaceutical formulations may contain preservatives, solubilizers, stabilizers, re-wetting agents, emulgators, sweeteners, dyes, adjusters, and salts for the adjustment of osmotic pressure, buffers, coating agents or antioxidants. Formulations comprising the compound may also contain other substances which have valuable therapeutic properties. Pharmaceutical formulations may be prepared by known pharmaceutical methods. Suitable formulations can be found, e.g., in ington's Pharmaceutical Sciences, Mack Publishing Company, Philadelphia, Pa.20th ed. (2000), which is incorporated herein by reference.

A compound detailed herein, or a pharmaceutically acceptable salt thereof, may be administered to individuals in a form of generally accepted oral compositions, such as tablets, coated tablets, and gel capsules in a hard or in soft shell, emulsions or suspensions. Examples of carriers, which may be used for the preparation of such compositions, are lactose, corn starch or its derivatives, talc, stearate or its salts, etc. Acceptable carriers for gel capsules with soft shell are, for instance, plant oils, wax, fats, semisolid and liquid poly-ols, and so on. In addition, pharmaceutical formulations may contain preservatives, solubilizers, stabilizers, re-wetting agents, emulgators, sweeteners, dyes, adjusters, and salts for the adjustment of osmotic pressure, buffers, coating agents or antioxidants.

Any of the compounds described herein can be formulated in a tablet in any dosage form described, for example, a compound as described herein or a salt thereof can be formulated as a 10 mg tablet.

Compositions comprising a compound provided herein are also described. In one variation, the composition comprises a compound, or a pharmaceutically acceptable salt thereof, and a pharmaceutically acceptable carrier or excipient. In another variation, a composition of substantially pure compound is provided. In some embodiments, the composition is for use as a human or veterinary medicament. In some embodiments, the composition is for use in a method described herein. In some embodiments, the composition is for use in the treatment of a disease or disorder described herein.

Compositions formulated for co-administration of a compound provided herein and one or more additional pharmaceutical agents are also described. The co-administration can be simultaneous or sequential in any order. A compound provided herein may be formulated for co-administration with the one or more additional pharmaceutical agents in the same dosage form (e.g., single tablet or single i.v.) or separate dosage forms (e.g., two separate tablets, two separate i.v., or one tablet and one i.v.). Furthermore, co-administration can be, for example, 1) concurrent delivery, through the same route of delivery (e.g., tablet or i.v.), 2) sequential delivery on the same day, through the same route or different routes of delivery, or 3) delivery on different days, through the same route or different routes of delivery.

### Methods of Use

Compounds and compositions detailed herein, such as a pharmaceutical composition containing a compound of any formula provided herein, or a pharmaceutically acceptable salt thereof, and a pharmaceutically acceptable carrier or excipient, may be used in methods of administration and treatment as provided herein. The compounds and compositions may also be used in in vitro methods, such as in vitro methods of administering a compound or composition to cells for screening purposes and/or for conducting quality control assays.

Provided herein is a method of treating a disease or disorder in an individual in need thereof comprising administering a compound described herein or any embodiment, variation, or aspect thereof, or a pharmaceutically acceptable salt thereof. In some embodiments, the compound, pharmaceutically acceptable salt thereof, or composition is administered to the individual according to a dosage and/or method of administration described herein.

In some embodiments, provided herein is a method of inhibiting FGFR4 in a cell or in an individual in need thereof comprising administering an effective amount of a compound or composition of the disclosure to the cell or individual. In some variations, the compounds provided herein are selective for inhibiting FGFR4 over FGFR1. As such, in some embodiments, provided herein is a method of selectively inhibiting FGFR4, as compared to FGFR1, in a cell or in an individual in need thereof comprising administering an effective amount of a compound or composition of the disclosure to the cell or individual.

In some embodiments, provided herein is a method for treating a condition mediated by FGFR4 activity comprising administering to an individual in need of treatment an effective amount of a compound of formula (I) or any related formula such as formula (I-a), (I-b), (I-c), (I-d), (I-e), (II), (II-a), (II-b), (II-c), (II-d), (II-e), (III), (III-a), (III-b), (III-c), (III-d), (III-e), (IV), (IV-a), (IV-b), (IV-c),(IV-d), (IV-e), (V), (VI), or (VI-a), or a pharmaceutically acceptable salt thereof. In some embodiments, the condition is cancer, such as a liver cancer.

In some embodiments, provided is a method for treating cancer, comprising administering to an individual in need thereof an effective amount of a compound of formula (I) or any related formula such as formula (I-a), (I-b), (I-c),(I-d), (I-e), (II), (II-a), (II-b), (II-c), (II-d), (II-e), (III), (III-a), (III-b), (III-c), (III-d), (III-e), (IV), (IV-a), (IV-b), (IV-c),(IV-d), (IV-e), (V), (VI), or (VI-a), or a pharmaceutically acceptable salt thereof. In some embodiments, the cancer is liver, colorectal, anal, breast, gastrointestinal, skin, stomach, esophageal, or pancreatic cancer. In some embodiments, the cancer is liver cancer. In some embodiments, the cancer originated from the liver or spread to the liver. In some embodiments, the cancer is hepatocellular carcinoma (HCC).

In one aspect, provided herein is a method of treating cancer, wherein modulation of FGFR4 activity prevents, inhibits, or ameliorates the pathology and/or symptomology of the cancer, in an individual, comprising administering to the individual a therapeutically effective amount of a compound or composition provided herein. In one embodiment, provided herein is a method of treating cancer, wherein modulation of FGFR4 activity prevents the pathology and/or symptomology of the cancer, in an individual, comprising administering to the individual a therapeutically effective amount of a compound or composition provided herein. In one embodiment, provided herein is a method of treating cancer, wherein modulation of FGFR4 activity inhibits the pathology and/or symptomology of the cancer, in an individual, comprising administering to the individual a therapeutically effective amount of a compound or composition provided herein. In one embodiment, provided herein is a method of treating a disease, wherein modulation of FGFR4 activity ameliorates the pathology and/or symptomology of the cancer, in an individual, comprising administering to the individual a therapeutically effective amount of a compound or composition provided herein.

In another aspect, provided herein is a method of delaying the onset and/or development of a cancer that is mediated by FGFR4 activity in an individual (such as a human) who is at risk for developing the cancer. It is appreciated that delayed development may encompass prevention in the event the individual does not develop the cancer.

In one aspect, provided herein is a method of delaying the onset and/or development of cancer in an individual in need thereof, comprising administering to the individual a therapeutically effective amount of a compound or composition provided herein. In some embodiments, the cancer is liver, colorectal, anal, breast, gastrointestinal, skin, stomach, esophageal, or pancreatic cancer. In some embodiments, the cancer originated from the liver or spread to the liver. In one aspect, provided herein is a method of delaying the onset and/or development of liver cancer in an individual in need thereof, comprising administering to the individual a therapeutically effective amount of a compound or composition provided herein. In one variation, provided herein is a method of delaying the onset and/or development of cancer that originated in the liver in an individual in need thereof, comprising administering to the individual a therapeutically effective amount of a compound or composition provided herein. In one variation, provided herein is a method of delaying the onset and/or development of cancer that spread to the liver in an individual in need thereof, comprising administering to the individual a therapeutically effective amount of a compound or composition provided herein. In one aspect, provided herein is a method of delaying the onset and/or development of hepatocellular carcinoma (HCC) in an individual in need thereof, comprising administering to the individual a therapeutically effective amount of a compound or composition provided herein.

In one aspect, provided herein is a compound of formula (I) or any variation thereof, or a pharmaceutically acceptable salt thereof, for use in therapy. In some embodiments, provided herein is a compound of formula (I) or any variation thereof, or a pharmaceutically acceptable salt thereof or pharmaceutical composition comprising such compound or a pharmaceutically acceptable salt thereof, for use in the treatment of cancer. In some embodiments, provided is a compound of formula (I) or any variation thereof, or a pharmaceutically acceptable salt thereof, or a pharmaceutical composition comprising such compound or a pharmaceutically acceptable salt thereof, for use in the treatment of liver, colorectal, anal, breast, gastrointestinal, skin, stomach, esophageal, or pancreatic cancer. In some embodiments, provided is a compound of formula (I) or any variation thereof, or a pharmaceutically acceptable salt thereof, or a pharmaceutical composition comprising such compound or a pharmaceutically acceptable salt thereof, for use in the treatment of liver cancer. In some embodiments, provided is a compound of formula (I) or any variation thereof, or a pharmaceutically acceptable salt thereof, or a pharmaceutical composition comprising such compound or a pharmaceutically acceptable salt thereof, for use in the treatment of a cancer which originates from the liver or spreads to the liver. In some embodiments, provided is a compound of formula (I) or any variation thereof, or a pharmaceutically acceptable salt thereof, or a pharmaceutical composition comprising such compound or a pharmaceutically acceptable salt thereof, for use in the treatment of hepatocellular carcinoma (HCC).

In another embodiment, provided herein is a compound of formula (I) or any variation thereof, or a pharmaceutically acceptable salt thereof, for use in the manufacture of a medicament for the treatment of cancer. In another embodiment, provided herein is a compound of formula (I) or any variation thereof, or a pharmaceutically acceptable salt thereof, for use in the manufacture of a medicament for the treatment of liver, colorectal, anal, breast, gastrointestinal, skin, stomach, esophageal, or pancreatic cancer. In some embodiments, the medicament is for the treatment of liver cancer. In some embodiments, the medicament is for the treatment of a cancer which originates from the liver or spreads to the liver. In some embodiments, the medicament is for the treatment of hepatocellular carcinoma (HCC).

In some embodiments, the individual is a mammal. In some embodiments, the individual is a primate, dog, cat, rabbit, or rodent. In some embodiments, the individual is a primate. In some embodiments, the individual is a human. In some embodiments, the human is at least about or is about any of 18, 21, 30, 50, 60, 65, 70, 75, 80, or 85 years old. In some embodiments, the human is a child. In some embodiments, the human is less than about or about any of 21, 18, 15, 10, 5, 4, 3, 2, or 1 years old.

In some embodiments, the method further comprises administering one or more additional pharmaceutical agents. In some embodiments, the method further comprises administering radiation. In some embodiments, the method further comprises administering one or more additional pharmaceutical agents and radiation.

In some embodiments, the method further comprises administering a platinum-based agent. In some embodiments, the method further comprises administering oxaliplatin or cisplatin. In some embodiments, the method further comprises administering a topoisomerase I inhibitor. In some embodiments, the method further comprises administering irinotecan. In some embodiments, the method further comprises administering mitomycin and/or methotrexate. In some embodiments, the method further comprises administering mitomycin. In some embodiments, the method further comprises administering methotrexate.

In some embodiments, the method further comprises administering a taxane. In some embodiments, the method further comprises administering a taxane and a platinum-based agent. In some embodiments, the method further comprises administering docetaxel or paclitaxel.

In some embodiments, the method further comprises administering one or more additional pharmaceutical agents which are useful for treating liver cancer, such as pharmaceutical agents disclosed in Villanueva, A. (2019) N. Engl. J. Med., 380:1450-62. In some embodiments, the method further comprises administering one or more additional pharmaceutical agents which are cabozantinib-S-malate, pembrolizumab, lenvatinib mesylate, sorafenib tosylate, nivolumab, ramucirumab, regorafenib, or combinations thereof. In some embodiments, the method further comprises administering cabozantinib-S-malate. In some embodiments, the method further comprises administering pembrolizumab. In some embodiments, the method further comprises administering lenvatinib mesylate. In some embodiments, the method further comprises administering sorafenib tosylate. In some embodiments, the method further comprises administering nivolumab. In some embodiments, the method further comprises administering regorafenib. In some embodiments, the method further comprises administering ramucirumab.

### Dosing and Method of Administration

The dose of a compound described herein, or a stereoisomer, tautomer, solvate, or salt thereof, administered to an individual (such as a human) may vary with the particular compound or salt thereof, the method of administration, and the particular cancer, such as type and stage of cancer, being treated. In some embodiments, the amount of the compound, or a stereoisomer, tautomer, solvate, or salt thereof, is a therapeutically effective amount.

The compounds provided herein or a salt thereof may be administered to an individual via various routes, including, e.g., intravenous, intramuscular, subcutaneous, oral, and transdermal.

The effective amount of the compound may in one aspect be a dose of between about 0.01 and about 100 mg/kg. Effective amounts or doses of the compounds of the present disclosure may be ascertained by routine methods, such as modeling, dose escalation, or clinical trials, taking into account routine factors, e.g., the mode or route of administration or drug delivery, the pharmacokinetics of the agent, the severity and course of the disease to be treated, the subject's health status, condition, and weight. An exemplary dose is in the range of about from about 0.7 mg to 7 g daily, or about 7 mg to 350 mg daily, or about 350 mg to 1.75 g daily, or about 1.75 to 7 g daily.

Any of the methods provided herein may in one aspect comprise administering to an individual a pharmaceutical composition that contains an effective amount of a compound provided herein, or a stereoisomer, tautomer, solvate, or salt thereof, and a pharmaceutically acceptable excipient.

A compound or composition provided herein may be administered to an individual in accordance with an effective dosing regimen for a desired period of time or duration, such as at least about one month, at least about 2 months, at least about 3 months, at least about 6 months, or at least about 12 months or longer, which in some variations may be for the duration of the individual's life. In one variation, the compound is administered on a daily or intermittent schedule. The compound can be administered to an individual continuously (for example, at least once daily) over a period of time. The dosing frequency can also be less than once daily, e.g., about a once weekly dosing. The dosing frequency can be more than once daily, e.g., twice or three times daily. The dosing frequency can also be intermittent, including a ‘drug holiday’ (e.g., once daily dosing for 7 days followed by no doses for 7 days, repeated for any 14 day time period, such as about 2 months, about 4 months, about 6 months or more). Any of the dosing frequencies can employ any of the compounds described herein together with any of the dosages described herein.

### Articles of Manufacture and Kits

The present disclosure further provides articles of manufacture comprising a compound described herein or a salt thereof, a composition described herein, or one or more unit dosages described herein in suitable packaging. In certain embodiments, the article of manufacture is for use in any of the methods described herein. Suitable packaging is known in the art and includes, for example, vials, vessels, ampules, bottles, jars, flexible packaging and the like. An article of manufacture may further be sterilized and/or sealed.

The present disclosure further provides kits for carrying out the methods of the present disclosure, which comprises one or more compounds described herein or a composition comprising a compound described herein. The kits may employ any of the compounds disclosed herein. In one variation, the kit employs a compound described herein or pharmaceutically acceptable salt thereof. The kits may be used for any one or more of the uses described herein, and, accordingly, may contain instructions for the treatment of any disease or described herein, for example for the treatment of cancer, including liver, colorectal, anal, breast, gastrointestinal, skin, stomach, esophageal, and pancreatic cancer. In some embodiments, the cancer originated from the liver or spread to the liver. In some embodiments, the cancer is hepatocellular carcinoma (HCC).

The kits optionally further comprise a container comprising one or more additional pharmaceutical agents and which kits further comprise instructions on or in the package insert for treating the subject with an effective amount of the one or more additional pharmaceutical agents. The one or more additional pharmaceutical agents may be cabozantinib-S-malate, pembrolizumab, lenvatinib mesylate, sorafenib tosylate, nivolumab, ramucirumab, regorafenib, or combinations thereof. The one or more additional pharmaceutical agents may be cabozantinib-S-malate. The one or more additional pharmaceutical agents may be pembrolizumab. The one or more additional pharmaceutical agents may be lenvatinib mesylate. The one or more additional pharmaceutical agents may be sorafenib tosylate. The one or more additional pharmaceutical agents may be nivolumab. I The one or more additional pharmaceutical agents may be regorafenib. In some embodiments, the method further comprises administering ramucirumab.

Kits generally comprise suitable packaging. The kits may comprise one or more containers comprising any compound described herein. Each component (if there is more than one component) can be packaged in separate containers or some components can be combined in one container where cross-reactivity and shelf life permit.

The kits may be in unit dosage forms, bulk packages (e.g., multi-dose packages) or sub-unit doses. For example, kits may be provided that contain sufficient dosages of a compound as disclosed herein and/or an additional pharmaceutically active compound useful for a disease detailed herein to provide effective treatment of an individual for an extended period, such as any of a week, 2 weeks, 3 weeks, 4 weeks, 6 weeks, 8 weeks, 3 months, 4 months, 5 months, 7 months, 8 months, 9 months, or more. Kits may also include multiple unit doses of the compounds and instructions for use and be packaged in quantities sufficient for storage and use in pharmacies (e.g., hospital pharmacies and compounding pharmacies).

The kits may optionally include a set of instructions, generally written instructions, although electronic storage media (e.g., magnetic diskette or optical disk) containing instructions are also acceptable, relating to the use of component(s) of the methods of the present disclosure. The instructions included with the kit generally include information as to the components and their administration to an individual.

### General Synthetic Methods

The compounds of the present disclosure may be prepared by a number of processes as generally described below and more specifically in the Examples hereinafter (such as the schemes provided in the Examples below). In the following process descriptions, the symbols when used in the formulae depicted are to be understood to represent those groups described above in relation to the formulae herein.

The intermediates described in the following preparations may contain a number of nitrogen, hydroxy, and acid protecting groups such as esters. The variable protecting group may be the same or different in each occurrence depending on the particular reaction conditions and the particular transformations to be performed. The protection and deprotection conditions are well known to the skilled artisan and are described in the literature. See. e.g., Greene and Wuts, Protective Groups in Organic Synthesis, (T. Greene and P. Wuts, eds., 2d ed. 1991).

Certain stereochemical centers have been left unspecified and certain substituents have been eliminated in the following schemes for the sake of clarity and are not intended to limit the teaching of the schemes in any way. Furthermore, individual isomers, enantiomers, and diastereomers may be separated or resolved by one of ordinary skill in the art at any convenient point in the synthesis of compounds of the invention, by methods such as selective crystallization techniques or chiral chromatography (See for example, J. Jacques, et al., “Enantiomers, Racemates, and Resolutions”, John Wiley and Sons, Inc., 1981, and E. L. Eliel and S. H. Wilen, “Stereochemistry of Organic Compounds”, Wiley-Interscience, 1994).

The compounds of the present invention, or salts thereof, may be prepared by a variety of procedures known in the art, some of which are illustrated in the Examples below. The specific synthetic steps for each of the routes described may be combined in different ways, to prepare compounds of the invention, or salts thereof. The products of each step can be recovered by conventional methods well known in the art, including extraction, evaporation, precipitation, chromatography, filtration, trituration, and crystallization. The reagents and starting materials are readily available to one of ordinary skill in the art. Others may be made by standard techniques of organic and heterocyclic chemistry which are analogous to the syntheses of known structurally-similar compounds and the procedures described in the Examples which follow including any novel procedures.

Compounds of formula (I) can be prepared according to Scheme 1, wherein the Ring A moiety, L, Y, n, V, W, R¹, R⁵, and R⁶ are as defined for formula (I), or any applicable variations detailed herein.

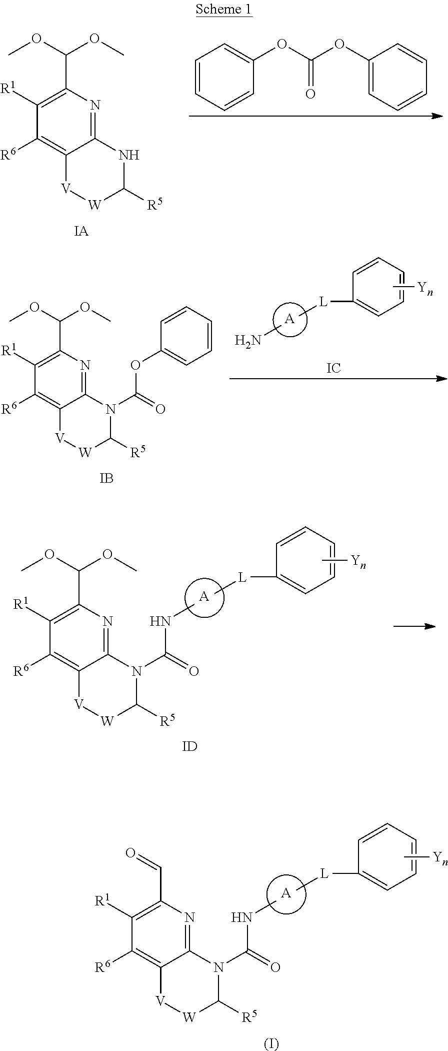

Acylation of amines of general formula IA with diphenyl carbonate yields carbamates of general formula IB, which can be reacted with amines of general formula IC to yield carbamides of general formula ID. Subsequent deprotection via removal of the acetal group gives the compound of formula (I).

Compounds of general formula IA can be prepared according to Scheme 2, wherein V, W, R¹, R⁵, and R⁶ are as defined for formula (I), or any applicable variations detailed herein, and X is a halogen (e.g., Cl, Br, or I).

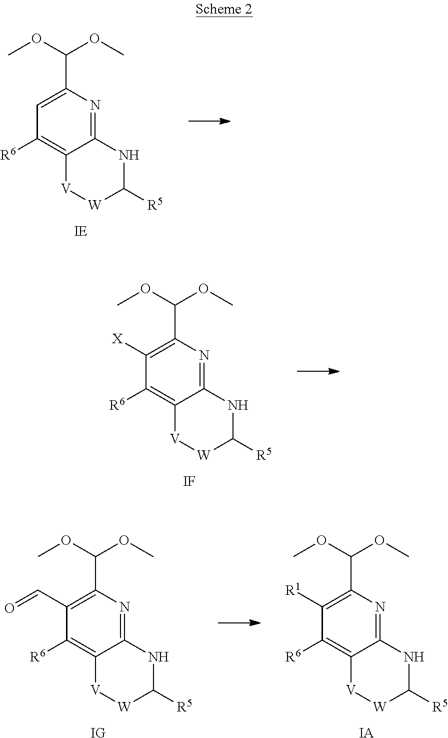

Halogenation of compounds of general formula IE yields compounds of general formula IF. Formylation of compounds of general formula IF gives compounds of general formula IG, which can further undergo reaction to give compounds of general formula IA.

Compounds of general formula IC-1 can be prepared according to Scheme 3, wherein the A Ring moiety, Y, and n are as defined for formula (I), or any applicable variations detailed herein; R is C₁-C₄ alkyl; X¹ is Cl, Br, I, OMs, OTs, or another suitable leaving group; and X² is Br or I.

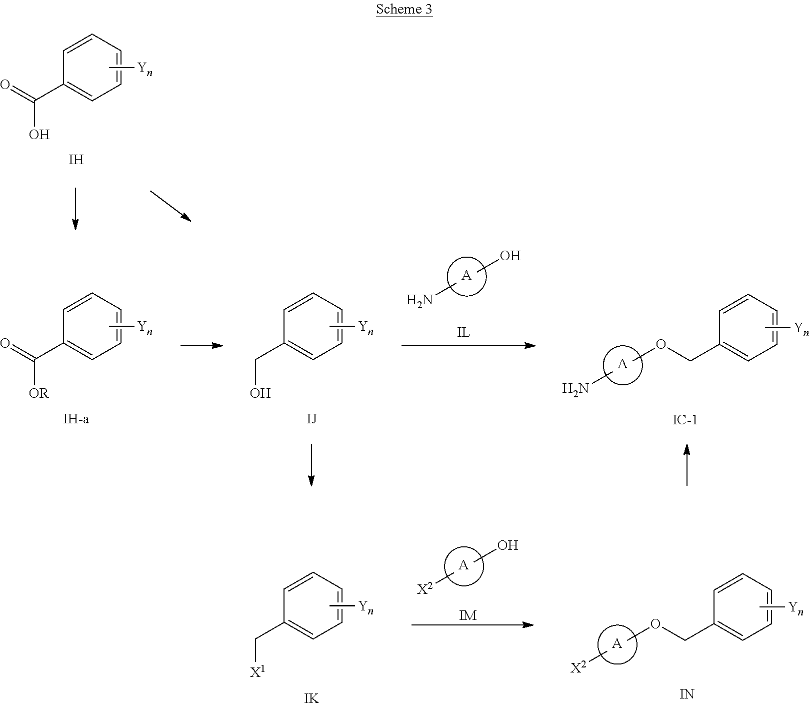

Esterification of carboxylic acids of general formula IH yields compounds of general formula IH-a, which can undergo reduction to yield the alcohols of general formula IJ. Alternatively, carboxylic acids of general formula IH can be directly reduced to alcohols of general formula IJ. Alcohols of general formula IJ can be converted to alkyl halides of general formula IK, which undergoes a nucleophilic substitution reaction with alcohols of general formula IM to yield ethers of general formula IN. Compounds of general formula IC-1 can be prepared from compounds of general formula IN. Alternatively, a dehydration reaction between alcohols of general formula IJ and alcohols of general formula IL yields compounds of general formula IC-1.

Compounds of general formulae IC-2, IC-3, or IC-4 can be prepared according to Scheme 4, wherein the Ring A moiety, Y, and n are as defined for formula (I), or any applicable variations detailed herein.

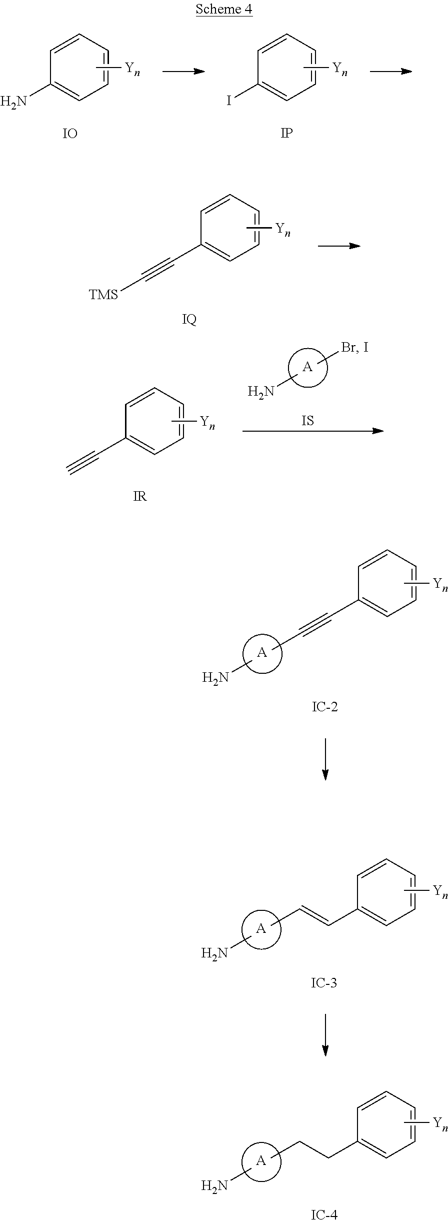

Aromatic substitution of anilines of general formula IO yields iodobenzenes of general formula IP, which can undergo alkynylation to yield compounds of general formula IQ. Subsequent deprotection of compounds of general formula IQ yields aryl acetylenes of general formula IR. Coupling of terminal alkynes of general formula IR with aryl halides or heteroaryl halides of general formula IS yields ethynes of general formula IC-2. Compounds of general formula IC-2 can undergo hydrogenation to yield ethenes of general formula IC-3, which can undergo further hydrogenation to yield ethanes of formula IC-4.

### EXAMPLES

It is understood that the present disclosure has been made only by way of example, and that numerous changes in the combination and arrangement of parts can be resorted to by those skilled in the art without departing from the spirit and scope of present disclosure.

The chemical reactions in the Examples described can be readily adapted to prepare a number of other compounds disclosed herein, and alternative methods for preparing the compounds of this disclosure are deemed to be within the scope of this disclosure. For example, the synthesis of non-exemplified compounds according to the present disclosure can be successfully performed by modifications apparent to those skilled in the art, e.g., by appropriately protecting interfering groups, by utilizing other suitable reagents known in the art other than those described, or by making routine modifications of reaction conditions, reagents, and starting materials. Alternatively, other reactions disclosed herein or known in the art will be recognized as having applicability for preparing other compounds of the present disclosure.

Abbreviations used in the Examples include the following: DCM: dichloromethane; DMF: dimethylformamide; DMSO: dimethyl sulfoxide; EtOAc: ethyl acetate; EtOH: ethanol or ethyl alcohol; ¹H NMR: proton nuclear magnetic resonance; LCMS: liquid chromatography-mass spectrometry; LiHMDS: lithium hexamethyldisilazide; MeOH: methanol or methyl alcohol; Py: pyridine; THF: tetrahydrofuran; and TLC: thin-layer chromatography.

### Example S1: Synthesis of N-(5-((2,6-difluoro-3,5-dimethoxybenzyl)oxy)pyrimidin-2-yl)-7-formyl-3,4-dihydro-1,8-naphthyridine-1(2H)-carboxamide

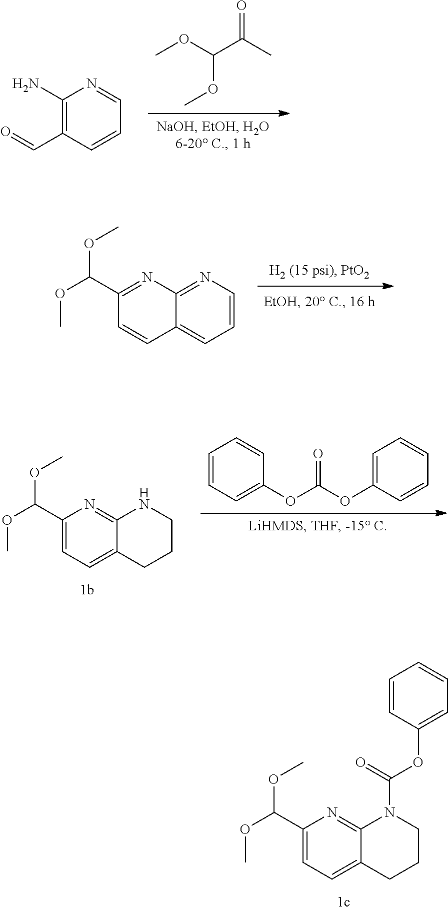

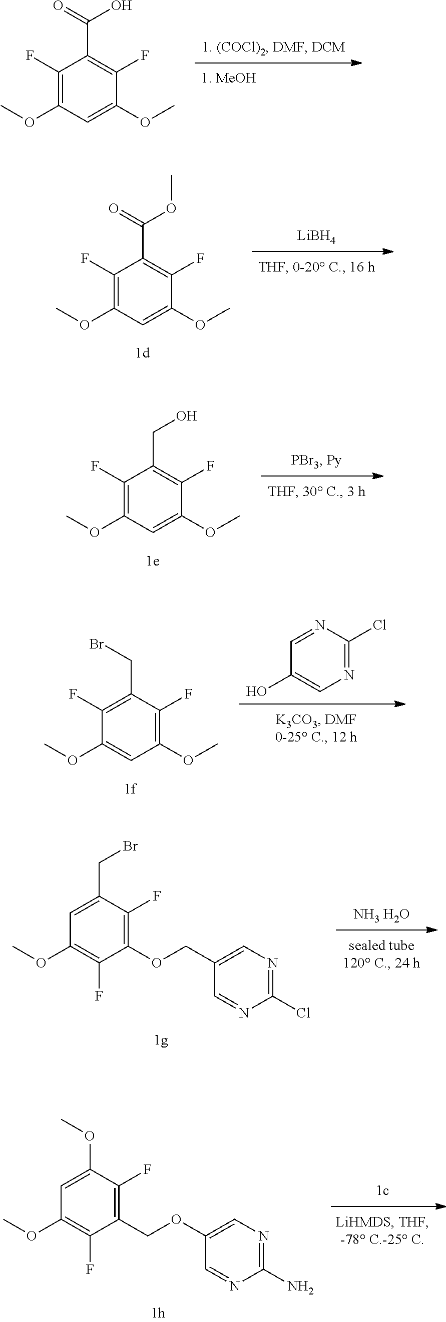

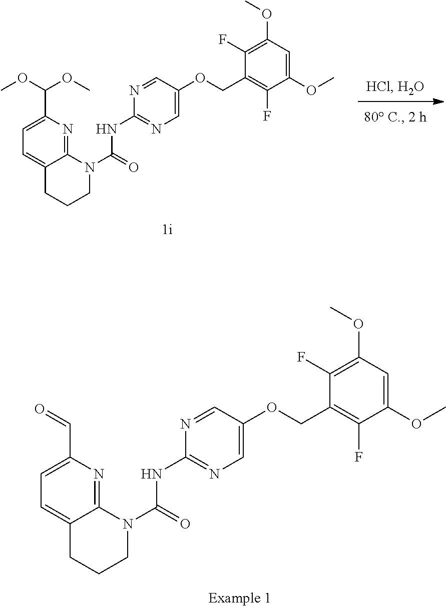

Synthesis of 2-(dimethoxymethyl)-1,8-naphthyridine (1a). To a mixture of 2-aminonicotinaldehyde (1 g, 8.19 mmol) and 1,1-dimethoxypropan-2-one (1.26 g, 10.64 mmol) in EtOH (15 mL) and H₂O (2.2 mL) was added a solution of NaOH (5 M, 2.13 mL) in one portion at 6° C. under N₂. The mixture was stirred at 20° C. for 1 hour. TLC (PE:EA=0:1, R_{f}=0.49) indicated reactant was consumed completely and one new spot formed. The reaction mixture was concentrated under reduced pressure to remove solvent. The residual was dissolved into EtOAc (50 mL) and washed with H₂O (20 mL*3) and brine (20 mL). The organic phase was concentrated under reduced pressure to give 1a. ¹H NMR (400 MHz, CDCl₃) δ 9.14 (dd, J=1.9, 4.3 Hz, 1H), 8.29-8.16 (m, 2H), 7.80 (d, J=8.4 Hz, 1H), 7.52 (dd, J=4.3, 8.0 Hz, 1H), 5.49 (s, 1H), 3.58-3.50 (m, 6H).

Synthesis of 7-(dimethoxymethyl)-1,2,3,4-tetrahydro-1,8-naphthyridine (1b). To a solution of 2-(dimethoxymethyl)-1,8-naphthyridine (1a) (1.4 g, 6.86 mmol) in EtOH (15 mL) was added PtO₂ (53.85 mg, 237.13 umol) under N₂. The suspension was degassed under vacuum and purged with H₂ several times. The mixture was stirred under H₂ (15 psi) at 20° C. for 16 hours. TLC (EtOAc, R_{f}=0.4) indicated la was consumed completely and one new spot formed. The mixture was filtered and the filtrate was concentrated under reduced pressure. The residue was purified by prep-TLC (SiO₂, EtOAc) to give 1b. ¹H NMR (400 MHz, CDCl₃) δ 7.17 (d, J=7.3 Hz, 1H), 6.71 (d, J=7.3 Hz, 1H), 5.11 (s, 1H), 3.44-3.39 (m, 2H), 3.38 (s, 6H), 2.73 (t, J=6.3 Hz, 2H), 1.98-1.85 (m, 2H).

Synthesis of phenyl 7-(dimethoxymethyl)-3,4-dihydro-1,8-naphthyridine-1(2H)-carboxylate (1c). To a mixture of 7-(dimethoxymethyl)-1,2,3,4-tetrahydro-1,8-naphthyridine (1b) (180 mg, 864.32 umol) and diphenyl carbonate (370.30 mg, 1.73 mmol) in THF (5 mL) was added LiHMDS (1 M, 1.21 mL) dropwise at −15° C. under N₂. The mixture was stirred at −15° C. for 30 min. TLC (EtOAc, R_{f}=0.8) indicated 1b was consumed completely and one new spot formed. The mixture was quenched with aq. NH₄Cl (10 mL) and extracted with EtOAc (15 mL*3). The combined organic phase was dried over Na₂SO₄ and concentrated. The residue was purified by column chromatography (SiO₂, petroleum ether : ethyl acetate from 10:1 to 5:1) to give 1c. ¹H NMR (400 MHz, DMSO-d6) δ 7.65 (d, J=7.7 Hz, 1H), 7.47-7.33 (m, 2H), 7.29-7.16 (m, 4H), 5.21-5.12 (m, 1H), 3.89-3.76 (m, 2H), 3.30-3.21 (m, 6H), 2.82 (t, J=6.5 Hz, 2H), 2.08-1.85 (m, 2H).

Synthesis of methyl 2,6-difluoro-3,5-dimethoxybenzoate (1d). To a solution of 2,6-difluoro-3,5-dimethoxybenzoic acid (400 mg, 1.83 mmol) in DCM (5 mL) was added DMF (13.40 mg, 183.36 umol) and (COCl)₂ (349.09 mg, 2.75 mmol) in one portion at 0° C. under N₂. The mixture was stirred at 25° C. for 1 hour. Next, MeOH (791.80 mg, 24.71 mmol) was added dropwise. TLC (petroleum ether:ethyl acetate=2:1, R_{f}=0.6) indicated one new spot formed. The reaction mixture was concentrated under reduced pressure to remove solvent. The residue was purified by column chromatography (SiO₂, petroleum ether:ethyl acetate from 1:0 to 10:1) to give 1d. ¹H NMR (400 MHz, CDCl₃) δ 6.74 (t, J=7.9 Hz, 1H), 3.97 (s, 3H), 3.90 (s, 6H).

Synthesis of (2,6-difluoro-3,5-dimethoxyphenyl)methanol (1e). To a solution of methyl 2,6-difluoro-3,5-dimethoxybenzoate (1d) (380 mg, 1.64 mmol) in THF (5 mL) was added LiBH₄ (106.96 mg, 4.91 mmol) in one portion at 0° C. under N2. The mixture was stirred at 20° C. for 16 hours. TLC (petroleum ether:ethyl acetate=2:1, R_{f}=0.4) indicated one new spot formed. The mixture was cooled to 0° C. and quenched with saturated NH₄Cl solution (1 mL). The mixture was concentrated under vacuum and dissolved into EtOAc (20 mL). The mixture was washed with H₂O (10 mL*2) and brine (10 mL), dried over Na₂SO₄ and concentrated. The residue was purified by column chromatography (SiO₂, petroleum ether: ethyl acetate from 10:1 to 4:1) to give 1e. ¹H NMR (400 MHz, CDCl₃) δ 6.63 (t, J=8.2 Hz, 1H), 4.81 (d, J=6.6 Hz, 2H), 3.89 (s, 6H).

Synthesis of 3-(bromomethyl)-2,4-difluoro-1,5-dimethoxybenzene (1f). To a mixture of (2,6-difluoro-3,5-dimethoxyphenyl)methanol (1e) (310 mg, 1.52 mmol) in THF (3 mL) was added pyridine (6.01 mg, 75.92 umol) and then PBr₃ (411.00 mg, 1.52 mmol) dropwise at 30° C. under N2. The mixture was stirred at 30° C. for 3 hours. TLC (petroleum ether: ethyl acetate=2:1, R_{f}=0.8) indicated one new spot was formed. The mixture was quenched with cold water and extracted with EtOAc (20 mL*3). The combined organic phase was washed with brine (20 mL) and dried over Na₂SO₄, then concentrated. The residue was purified by prep-TLC (SiO₂, petroleum ether:ethyl acetate=2:1) to give 1f. ¹H NMR (400 MHz, CDCl₃) δ 6.63 (t, J=8.2 Hz, 1H), 4.54 (t, J=1.4 Hz, 2H), 3.89 (s, 6H).

Synthesis of 2-chloro-5-((2,6-difluoro-3,5-dimethoxybenzyl)oxy)pyrimidine (1g). To a mixture of 2-chloropyrimidin-5-ol (73.31 mg, 561.66 umol) in DMF (2 mL) was added K₂CO₃ (194.06 mg, 1.40 mmol) in one portion at 0° C., then 3-(bromomethyl)-2,4-difluoro-1,5-dimethoxybenzene (1f) (150 mg, 561.66 umol) was added. The mixture was heated to 25° C. and stirred for 12 hours. TLC (petroleum ether:ethyl acetate=2:1, R_{f}=0.7) indicated that one new spot was formed. The mixture was poured into EtOAc (30 mL), washed with H₂O (10 mL*3) and brine (10 mL), then dried over Na₂SO₄ and concentrated. The residue was purified by prep-TLC (SiO₂, petroleum ether:ethyl acetate=3:1) to give 1g (150 mg, 473.65 umol, 84.33% yield) as a white solid. ¹H NMR (400 MHz, CDCl₃) δ 8.38 (s, 2H), 6.71 (t, J=8.2 Hz, 1H), 5.25 (t, J=1.4 Hz, 2H), 3.91 (s, 6H).

Synthesis of 5-((2,6-difluoro-3,5-dimethoxybenzyl)oxy)pyrimidin-2-amine (1h). A solution of 2-chloro-5-((2,6-difluoro-3,5-dimethoxybenzyl)oxy)pyrimidine (1g) (150 mg, 473.65 umol) in NH₃.H₂O (10 mL, 28%) was stirred at 120° C. for 24 hr in a sealed tube. TLC (petroleum ether:ethyl acetate=1:1, R_{f}=0.4) indicated that one new spot formed. The mixture was quenched with H₂O, and extracted with EtOAc (30 mL*4). The combined organic phase was washed with brine (20 mL), dried over Na₂SO₄ and concentrated. The residue was purified by prep-TLC (SiO₂, petroleum ether:ethyl acetate=1:1) to give 1h. ¹H NMR (400 MHz, CDCl₃) δ 8.11 (s, 2H), 6.68 (t, J=8.1 Hz, 1H), 5.12 (s, 1H), 5.24-4.97 (m, 1H), 4.82 (s, 2H), 3.99-3.79 (m, 6H).

Synthesis of N-(5-((2,6-difluoro-3,5-dimethoxybenzyl)oxy)pyrimidin-2-yl)-7-(dimethoxymethyl)-3,4-dihydro-1,8-naphthyridine-1(2H)-carboxamide (1i). To a solution of 5-((2,6-difluoro-3,5-dimethoxybenzyl)oxy)pyrimidin-2-amine (1h) (0.025 g, 84.10 umol) in THF (2 mL) cooled to −78° Cwas added LiHMDS (1 M, 168.20 uL) at −78° C., followed by addition of phenyl 7-(dimethoxymethyl)-3,4-dihydro-1,8-naphthyridine-1(2H)-carboxylate (1c) (30.38 mg, 92.51 umol) at −78° C. The mixture was stirred at 25° C. for 2 h. LCMS showed the reaction was completed. The residue was poured into water (2 mL). The aqueous phase was extracted with ethyl acetate (5 mL*2). The combined organic phase was washed with brine (2 mL*2), dried with anhydrous Na₂SO₄, filtered and concentrated in vacuum. The residue was purified by prep-TLC (petroleum ether:ethyl acetate=0:1, R_{f}=0.5) to give 1i. MS mass calculated for [M+H]⁺ (C₂₅H₂₇F₂N₅O₆) requires MS 532.2, MS found MS 532.1.

Synthesis of N-(5-((2,6-difluoro-3,5-dimethoxybenzyl)oxy)pyrimidin-2-yl)-7-formyl-3,4-dihydro-1,8-naphthyridine-1(2H)-carboxamide (Example 1). The solution of N-(5-((2,6-difluoro-3,5-dimethoxybenzyl)oxy)pyrimidin-2-yl)-7-(dimethoxymethyl)-3,4-dihydro-1,8-naphthyridine-1(2H)-carboxamide (1i) (30 mg, 56.44 umol) in HCl (1 mL) and H₂O (1 mL) was stirred at 80° C. for 2 h. LCMS showed the reaction was completed. The mixture was concentrated in vacuum to give Example 1. MS mass calculated for [M+H]⁺ (C₂₃H₂₁F₂N₅O₅) requires MS 486.4, MS found MS 486.1; ¹H NMR (400 MHz, DMSO-d₆) δ ppm 13.41 (s, 1 H) 9.92 (s, 1 H) 8.53 (s, 2 H) 7.91 (d, J=7.5 Hz, 1 H) 7.64 (d, J=7.5 Hz, 1 H) 7.09 (t, J=8.5 Hz, 1 H) 5.28 (s, 2 H) 3.95 (d, J=5.5 Hz, 2 H) 3.88 (s, 6 H) 2.94 (t, J=6.0 Hz, 2 H) 1.94 (s, 2 H).

### Example S2: Synthesis of N-(5-((2,6-difluoro-3,5-dimethoxybenzyl)oxy)pyridin-2-yl)-7-formyl-3,4-dihydro-1,8-naphthyridine-1(2H)-carboxamide

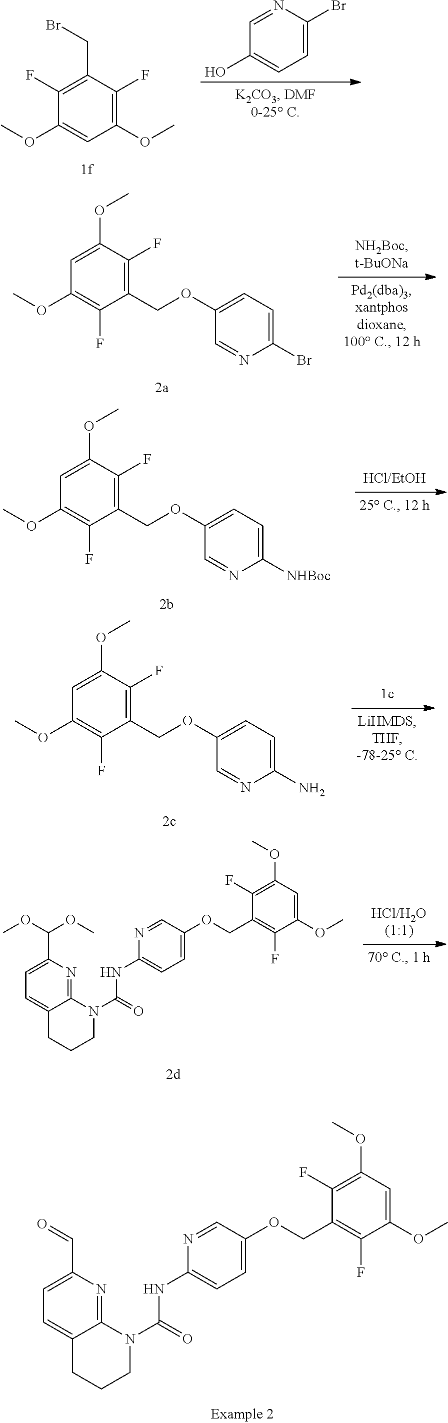

Synthesis of 2-bromo-5-((2,6-difluoro-3,5-dimethoxybenzyl)oxy)pyridine (2a). To a solution of 6-bromopyridin-3-ol (65.15 mg, 374.44 umol) in DMF (2 mL) was added K₂CO₃ (129.38 mg, 936.09 umol). The mixture was cooled to 0° C. and then 3-(bromomethyl)-2,4-difluoro-1,5-dimethoxybenzene (1f) (0.1 g, 374.44 umol) was added at 0° C. The mixture was then stirred at 25° C. for 12 h. LCMS showed the reaction was completed. The residue was poured into ice-water (5 mL) and stirred for 5 min. The mixture was filtered and the filter cake was concentrated in vacuum to give 2a. ¹H NMR (400MHz, CDCl₃) δ ppm 8.15 (d, J=3.1 Hz, 1 H) 7.39 (d, J=8.7 Hz, 1 H) 7.21 (dd, J=8.7, 3.2 Hz, 1 H) 6.69 (t, J=8.1 Hz, 1 H) 5.18 (t, J=1.4 Hz, 2 H) 3.87-3.94 (s, 6 H).

Synthesis of tert-butyl (5-((2,6-difluoro-3,5-dimethoxybenzyl)oxy)pyridin-2-yl)carbamate (2b). To a solution of 2-bromo-5-((2,6-difluoro-3,5-dimethoxybenzyl)oxy)pyridine (2a) (50 mg, 138.83 umol) and tert-butyl carbamate (16.26 mg, 138.83 umol) in dioxane (1 mL) was added t-BuONa (40.03 mg, 416.49 umol), Pd₂(dba)₃ (12.71 mg, 13.88 umol) and Xantphos (24.10 mg, 41.65 umol) under N₂. The mixture was stirred at 110° C. for 12 h. LCMS showed the reaction was completed. The residue was poured into water (2 mL). The aqueous phase was extracted with ethyl acetate (2 mL*2). The combined organic phase was washed with brine (1 mL*1), dried with anhydrous Na₂SO₄, filtered and concentrated in vacuum. The crude product was purified by prep-HPLC (column: Xtimate C18 150*25 mm*5 um; mobile phase: [water (10 mM NH₄HCO₃)-ACN]; B %: 25%-55%,10 min) to give 2b. ¹H NMR (400 MHz, CDCl₃) δ ppm 8.01 (d, J=2.8 Hz, 1 H) 7.87 (d, J=9.0 Hz, 1 H) 7.53(s, 1H) 7.35 (dd, J=9.1, 2.9 Hz, 1H) 6.67 (t, J=8.1 Hz, 1 H) 5.15 (s, 2 H) 3.84-3.95 (m, 6H) 1.53 (s, 9 H).

Synthesis of 5-((2,6-difluoro-3,5-dimethoxybenzyl)oxy)pyridin-2-amine (2c). The solution of tert-butyl (5-((2,6-difluoro-3,5-dimethoxybenzyl)oxy)pyridin-2-yl)carbamate (2b) (50 mg, 126.14 umol) in HCl/EtOAc (2 mL, 4M) was stirred at 25° C. for 12 h. LCMS showed the reaction was completed. The mixture was concentrated in vacuum to give 2c. MS mass calculated for [M+H]⁺ (C₁₄H₁₄F₂N₂O₃) requires MS 297.1, MS found MS 297.2.

Synthesis of N-(5-((2,6-difluoro-3,5-dimethoxybenzyl)oxy)pyridin-2-yl)-7-(dimethoxymethyl)-3,4-dihydro-1,8-naphthyridine-1(211)-carboxamide (2d). To a solution of 5-((2,6-difluoro-3,5-dimethoxybenzyl)oxy)pyridin-2-amine (2c) (20 mg, 60.11 umol) in THF (1 mL) was added LiHMDS (1 M, 120.22 uL) and phenyl 7-(dimethoxymethyl)-3,4-dihydro-1,8-naphthyridine-1(2H)-carboxylate (1c) (19.74 mg, 60.11 umol) under -78° C. The mixture was stirred at 25° C. for 2 h. LCMS showed the reaction was completed. The residue was poured into water (2 mL). The aqueous phase was extracted with ethyl acetate (5 mL*2). The combined organic phase was washed with brine (2 mL*2), dried with anhydrous Na₂SO₄, filtered and concentrated in vacuum. The residue was purified by prep-TLC (petroleum ether: ethyl acetate=0/1, R_{f}=0.5) to give 2d. MS mass calculated for [M+H]⁺ (C₂₆H₂₈F₂N₄O₆) requires MS 531.2, MS found MS 531.1.

Synthesis of N-(5-((2,6-difluoro-3,5-dimethoxybenzyl)oxy)pyridin-2-yl)-7-formyl-3,4-dihydro-1,8-naphthyridine-1(2H)-carboxamide (Example 2). A solution of N-(5-((2,6-difluoro-3,5-dimethoxybenzyl)oxy)pyridin-2-yl)-7-(dimethoxymethyl)-3,4-dihydro-1,8-naphthyridine-1(2H)-carboxamide (2d) (0.01 g, 18.85 umol) in H₂O (1 mL) and HCl (1 mL) was stirred at 70° C. for 1 h. LCMS showed the reaction was completed. The mixture was concentrated in vacuum. The crude product was purified by prep-HPLC (column: HUAPU C8 Extreme BDS 150*30*5u; mobile phase: [water (0.04%HC1)-ACN]; B%: 30%-60%, 11 min) to give Example 2. MS mass calculated for [M+H]⁺ (C₂₅H₂₇F₂N₅O₆) requires MS 485.3, MS found MS 485.2; ¹H NMR (400 MHz, DMSO-d6) δ ppm 9.97 (s, 1 H) 8.13 (s, 1 H) 8.03 (d, J=9.2 Hz, 1 H) 7.92 (d, J=7.8 Hz, 1 H) 7.66 (d, J=7.6 Hz, 1 H) 7.58 (dd, J=9.0, 2.8 Hz, 1 H) 7.09 (t, J=8.4 Hz, 1 H) 5.19 (s, 2 H) 3.98-4.03 (m, 2 H) 3.89 (s, 6 H) 2.97 (d, J=6.2 Hz, 3 H) 1.96 (s, 2 H).

Example S3: Synthesis of N-(5-((2,6-difluoro-3,5-dimethoxybenzyl)oxy)pyrimidin-2-yl)-7-formyl-6-((4-methyl-2-oxopiperazin-1-yl)methyl)-3,4-dihydro-1,8-naphthyridine-1(2H)-carboxamide

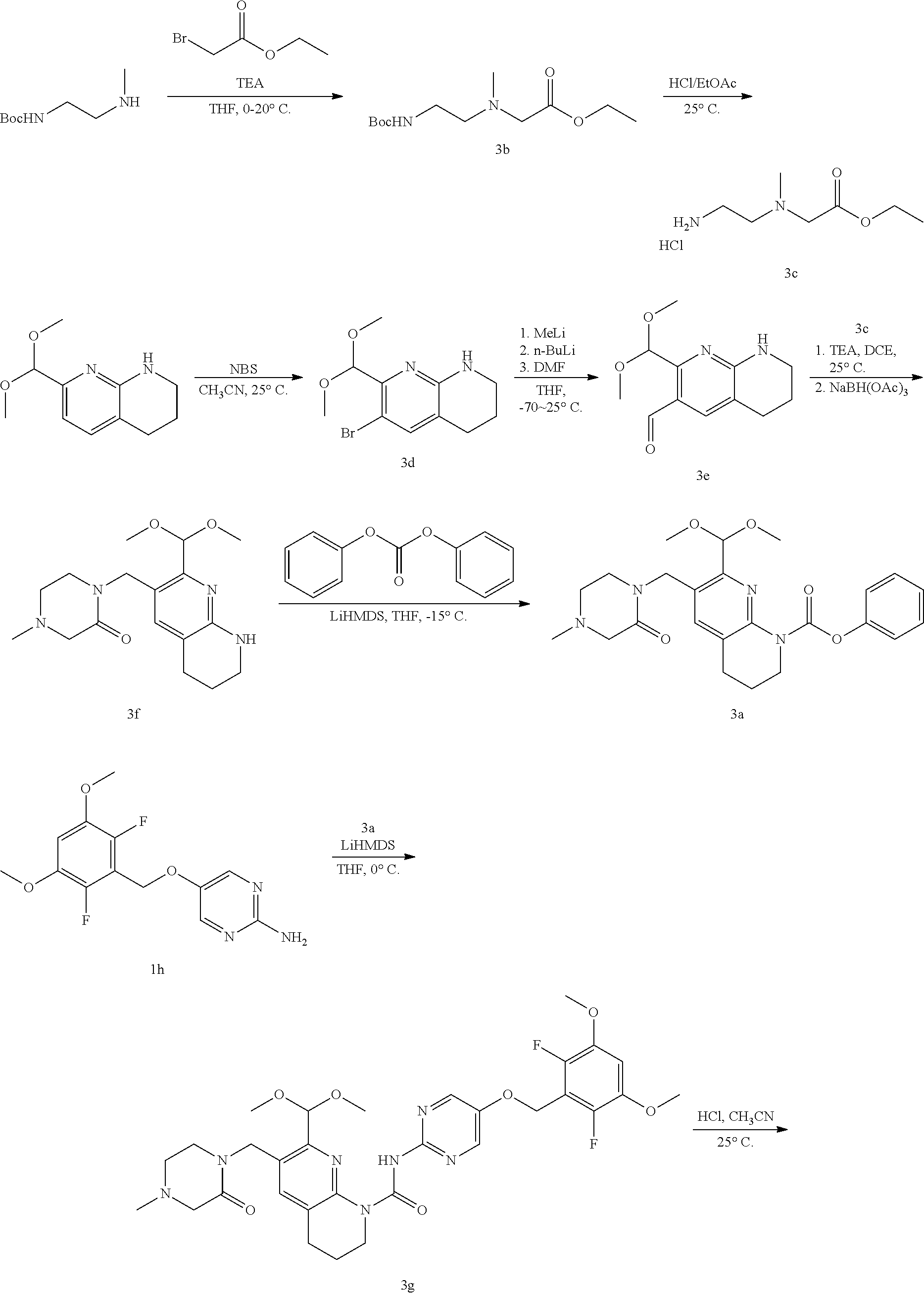

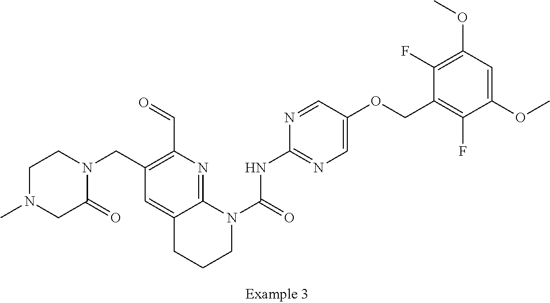

Synthesis of Ethyl 2-((2-((tert-butoxycarbonyl)amino)ethyl)(methyl)amino)acetate (3b). To a mixture of tert-butyl (2-(methylamino)ethyl)carbamate (3 g, 17.22 mmol) and TEA (5.23 g, 51.65 mmol, 7.19 mL) in THF (30 mL) was added ethyl 2-bromoacetate (2.88 g, 17.22 mmol, 1.90 mL) at 0° C. The reaction mixture was stirred at 20° C. for 18 hrs while White solid precipitated out. The reaction mixture was diluted with water (10 mL) and extracted with dichloromethane (15 mL*2). The combined organic phase was washed with saturated sodium bicarbonate solution (10 mL), dried over anhydrous Na₂SO₄, filtered and concentrated. The residue was purified by column chromatography (SiO₂, petroleum ether:ethyl acetate=10:1 to 1:1) to give 3b. ¹H NMR (400 MHz, CHLOROFORM-d) δ=5.18 (br s, 1H), 4.19 (q, J=7.3 Hz, 2H) , 3.27 (s, 2H), 3.21 (br d, J=5.9 Hz, 2H), 2.63 (t, J=5.9 Hz, 2H), 2.38 (s, 3H), 1.45 (s, 9H), 1.29 (t, J=7.1 Hz, 3H)

Synthesis of Ethyl 2-((2-aminoethyl)(methyl)amino)acetate hydrochloride (3c). A solution of Ethyl 2-((2-((tert-butoxycarbonyl) amino)ethyl)(methyl)amino)acetate (3b) (3.8 g, 14.60 mmol) in HCl/EtOAc (4 M, 20 mL) was stirred at 25° C. for 18 hrs. The reaction mixture was concentrated under reduced pressure to give 3c. ¹H NMR (400 MHz, DMSO-d₆) δ=8.44 (br s, 3H), 4.23 (q, J=6.8 Hz, 4H), 3.43 (br s, 2H), 3.25 (br s, 1H), 2.88 (br s, 3H), 1.26 (t, J=7.3 Hz, 3H)

Synthesis of 6-bromo-7-(dimethoxymethyl)-1,2,3,4-tetrahydro-1,8-naphthyridine (3d). To a solution of 7-(dimethoxymethyl)-1,2,3,4-tetrahydro-1,8-naphthyridine (2 g, 9.60 mmol) in CH₃CN (20 mL) was added NBS (1.71 g, 9.60 mmol) at 25° C. and stirred for 30 min. The reaction mixture was concentrated under reduced pressure. The residue was diluted with water (5 mL) and extracted with ethyl acetate (10 mL). The organic phase was washed with brine (5 mL), dried over Na₂SO₄, filtered, and concentrated under reduced pressure. The residue was purified by column chromatography (SiO₂, petroleum ether:ethyl acetate=10:1 to 1:1) to give 3d. ¹H NMR (400 MHz, CHLOROFORM-d) δ=5.56 (s, 1H), 5.11 (br s, 1H), 3.46 (s, 6H), 3.43-3.36 (m, 2H), 2.71 (br t, J=6.1 Hz, 2H), 1.88 (quin, J=5.9 Hz, 2H)

Synthesis of 2-(dimethoxymethyl)-5,6,7,8-tetrahydro-1,8-naphthyridine-3-carbaldehyde (3e). MeLi (1 M, 8.36 mL) was added drop-wise to a solution of 6-bromo-7-(dimethoxymethyl)-1,2,3,4-tetrahydro-1,8-naphthyridine (3d) (2 g, 6.96 mmol) in THF (20 mL) at −65˜−70° C. After the reaction solution was stirred at this temperature for 5 min, n-BuLi (2.5 M, 6.13 mL) was added dropwise, the resulting mixture was stirred for another 15 min, THF (15 mL) was added in. The reaction mixture was warmed up slowly to 25° C. and stirred for 30 min then DMF (2.04 g, 27.86 mmol, 2.14 mL) was added dropwise. After the addition was completed, the reaction mixture was cooled to 0° C. and quenched with saturated NH₄Cl aqueous solution (15 mL). The reaction mixture was stirred for 5 min, extracted with ethyl acetate (30 mL*2). The combined organic phase was washed with brine (10 mL), dried over anhydrous Na₂SO₄, filtered, and concentrated under reduced pressure. The residue was purified by column chromatography (SiO₂, petroleum ether:ethyl acetate=10:1 to 1:1), and then repurifed by column chromatography (SiO₂, dichloromethane:methanol=1:0 to 100:1) to give 3e. MS mass calculated for [M+H]⁺ (C₁₂H₁₆N₂O₃) requires m/z, 237.1, LCMS found m/z, 237.1; ¹H NMR (400 MHz, chloroform-d) δ=10.25 (s, 1H), 7.69 (s, 1H), 5.49 (s, 1H), 5.38 (s, 1H), 3.42 (br d, J=2.9 Hz, 2H), 3.39 (s, 6H), 2.69 (br t, J=6.1 Hz, 2H), 1.91-1.79 (m, 2H)

Synthesis of 1-((2-(dimethoxymethyl)-5,6,7,8-tetrahydro-1,8-naphthyridin-3-yl)methyl)-4-methylpiperazin-2-one (3f). 2-(dimethoxymethyl)-5,6,7,8-tetrahydro-1,8-naphthyridine-3-carbaldehyde (3e) (500 mg, 2.12 mmol), ethyl 2-((2-aminoethyl)(methyl)amino)acetate hydrochloride(3c) (541.08 mg, 2.75 mmol) and TEA (1.07 g, 10.58 mmol, 1.47 mL) was dissolved in 1,2-dichloroethane (2 mL) and the mixture was stirred for 1 hr. Then sodium triacetoxyborohydride (493.37 mg, 2.33 mmol) was added to the mixture and the mixture was stirred at 25° C. for 6 hrs. More sodium triacetoxyborohydride (493.37 mg, 2.33 mmol) was added and the mixture was stirred for another 48 hrs. The reaction mixture was diluted with water (10 mL) and extracted with dichloromethane (20 mL). The organic phase was washed with brine (5 mL), dried over Na₂SO₄, filtered, and concentrated under reduced pressure. The residue was purified by column chromatography (SiO₂, petroleum ether:ethyl acetate=1:1 to 0:1, then dichloromethane:methanol=10:1), repurified by Prep-TLC (dichloromethane:methanol=10:1, R_{f}=0.36) to give 3f. MS mass calculated for [M+H]⁺ (C₁₇H₂₆N₄O₃) requires m/z, 335.2, LCMS found m/z, 335.2; ¹H NMR (400 MHz, CHLOROFORM-d) δ=7.09 (s, 1H), 5.19 (s, 1H), 4.96 (br s, 1H), 4.71 (s, 2H), 3.41 (s, 6H), 3.40-3.36 (m, 2H), 3.21 (t, J=5.6 Hz, 2H), 3.18 (s, 2H), 2.69 (br t, J=6.1 Hz, 2H), 2.58 (t, J=5.6 Hz, 2H), 2.33 (s, 3H), 1.89 (td, J=6.0, 11.5 Hz, 2H)

Synthesis of Phenyl 7-(dimethoxymethyl)-6-((4-methyl-2-oxopiperazin-1-yl)methyl)-3,4-dihydro-1,8-naphthyridine-1(2H)-carboxylate (3a). To a solution of 1-((2-(dimethoxymethyl)-5,6,7,8-tetrahydro-1,8-naphthyridin-3-yl)methyl)-4-methylpiperazin-2-one (3f) (280 mg, 837.29 umol) and diphenyl carbonate (269.04 mg, 1.26 mmol) in THF (5 mL) was added LiHMDS (1 M, 1.67 mL) dropwise at −15° C. under N2 and stirred for 3 hrs. The reaction mixture was added to saturated NH₄Cl aqueous solution (10 mL) dropwise. The mixture was extracted with ethyl acetate (10 mL*3). The combined organic phase was washed with brine (10 mL), dried over Na₂SO₄, filtered, and concentrated under reduced pressure. The residue was purified by prep-HPLC (neutral condition, column: Welch Xtimate C18 250*50 mm*10 um; mobile phase: [water (10 mM NH₄HCO₃)-ACN]; B %: 25%-55%, 10 min) to give 3a. MS mass calculated for [M+H]⁺ (C₂₄H₃₀N₄O₅) requires m/z, 455.2, LCMS found m/z, 455.2; ¹H NMR (400 MHz, CHLOROFORM-d) δ=7.43 (s, 1H), 7.41-7.33 (m, 2H), 7.22 (m, 3H), 5.23 (s, 1H), 4.89 (s, 2H), 4.01-3.89 (m, 2H), 3.39 (s, 6H), 3.28 (br t, J=5.4 Hz, 2H), 3.22 (s, 2H), 2.82 (br t, J=6.6 Hz, 2H), 2.62 (br t, J=5.4 Hz, 2H), 2.36 (s, 3H), 2.09-1.97 (m, 2H)

Synthesis of N-(5-((2,6-difluoro-3,5-dimethoxybenzyl)oxy)pyrimidin-2-yl)-7-(dimethoxymethyl)-6-((4-methyl-2-oxopiperazin-1-yl)methyl)-3,4-dihydro-1,8-naphthyridine-1(2H)-carboxamide (3g). To a solution of 5-((2,6-difluoro-3,5-dimethoxybenzyl)oxy)pyrimidin-2-amine (1h) (43.16 mg, 145.21 umol) in THF (1 mL) was added LiHMDS (1 M, 290.42 uL) dropwise at 0° C. Then phenyl 7-(dimethoxymethyl)-6-((4-methyl-2-oxopiperazin-1-yl)methyl)-3,4-dihydro-1,8-naphthyridine-1(2H)-carboxylate (3a) (60 mg, 132.01 umol) dissolved in THF (1 mL) was added dropwise to the mixture at 0° C. and the mixture was stirred for 4 hrs. The reaction mixture was added to saturate NH₄Cl aqueous solution (5 mL) and the mixture was extracted with dichloromethane (10 mL*3). The combined organic phase was washed with brine (10 mL), dried over Na₂SO₄, filtered, and concentrated under reduced pressure. The residue was purified by prep-HPLC (neutral condition, column: Waters Xbridge BEH C18 100*30 mm*10 um; mobile phase: water (10 mM NH₄HCO₃)-ACN B%: 30%-60%, 10 min) to give 3g. MS mass calculated for [M+H]⁺ (C₃₁H₃₇F₂N₇O₇) requires m/z, 658.3, LCMS found m/z 658.2;

Synthesis of N-(5-((2,6-difluoro-3,5-dimethoxybenzyl)oxy)pyrimidin-2-yl)-7-formyl-6-((4-methyl-2-oxopiperazin-1-yl)methyl)-3,4-dihydro-1,8-naphthyridine-1(2H)-carboxamide (Example 3). To a solution of N-(5-((2,6-difluoro-3,5-dimethoxybenzyl)oxy)pyrimidin-2-yl)-7-(dimethoxymethyl)-6-((4-methyl-2-oxopiperazin-1-yl)methyl)-3,4-dihydro-1,8-naphthyridine-1(2H)-carboxamide (3g) (15 mg, 22.81 umol) in CH₃CN (2 mL) was added aqueous HCl (1 M, 0.5 mL) and the mixture was stirred at 25° C. for 2 hrs. The reaction mixture was concentrated under reduced pressure. The residue was purified by prep-HPLC (TFA condition; column: Welch Ultimate AQ-C18 150*30 mm*5 um; mobile phase: [water (0.1% TFA)-ACN]; B %: 10%-40%, 12 min) to give Example 3. MS mass calculated for [M+H]⁺ (C₂₉H₃₁F₂N₇O₆) requires m/z, 612.2, LCMS found m/z 612.3; ¹H NMR (400 MHz, CHLOROFORM-d) δ=13.71 (br s, 1H), 10.18 (s, 1H), 8.41 (s, 2H), 7.65 (s, 1H), 6.69 (br t, J=8.1 Hz, 1H), 5.22 (s, 2H), 5.13 (br s, 2H), 4.25-3.98 (m, 2H), 3.89 (s, 6H), 3.84 (br s, 2H), 3.71 (br s, 2H), 3.43 (br s, 2H), 2.94 (br t, J=6.1 Hz, 2H), 2.91-2.91 (m, 1H), 2.90 (s, 2H), 2.01 (br s, 2H)

### Example S4: Synthesis of N-(5-((2,6-difluoro-3,5-dimethoxybenzyl)oxy)pyrimidin-2-yl)-7-formyl-5-methyl-3,4-dihydro-1,8-naphthyridine-1(2H)-carboxamide

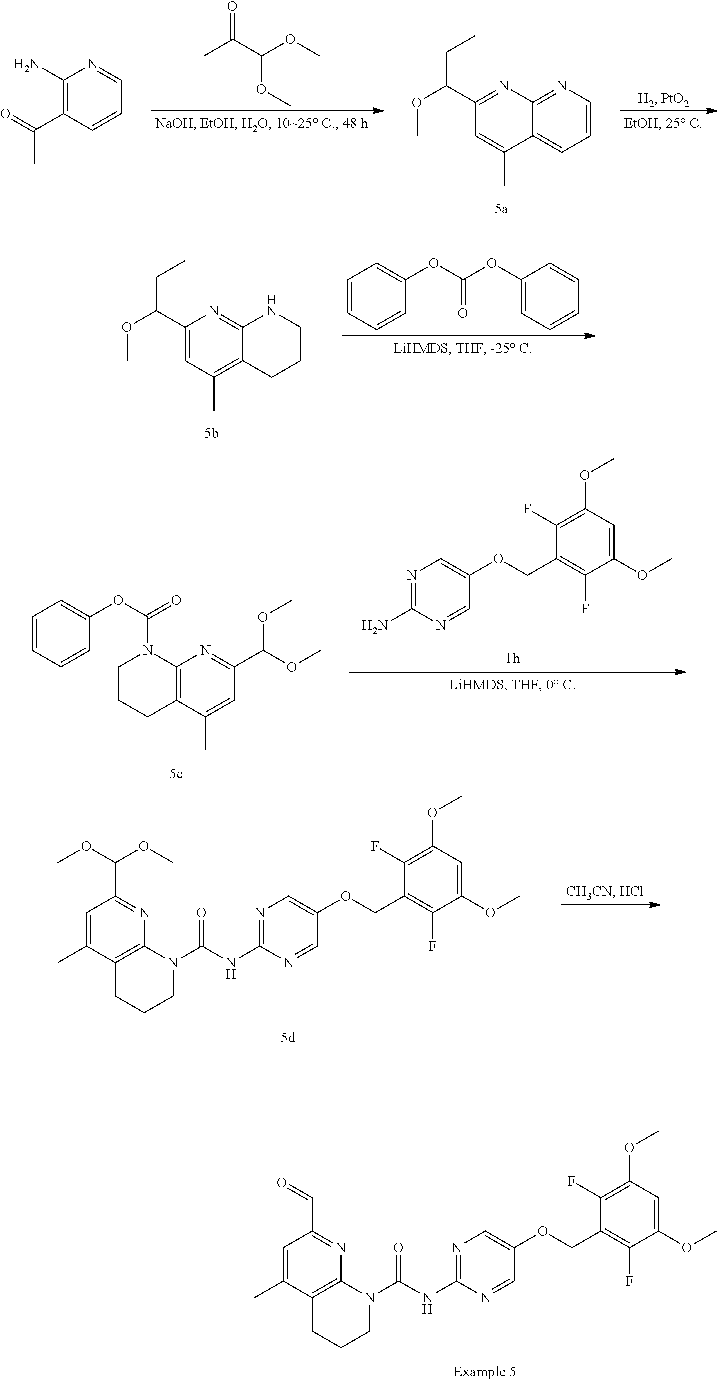

Synthesis of 2-(dimethoxymethyl)-4-methyl-1,8-naphthyridine (5a). To a solution of 1-(2-aminopyridin-3-yl) ethanone (1 g, 7.34 mmol), EtOH (20 mL), H₂O (4 mL) and 1,1-dimethoxypropan-2-one (2.17 g, 18.36 mmol) was added NaOH (587.58 mg, 14.69 mmol) in H₂O (4 mL) at a rate such that the internal temperature was lower than 10° C. The mixture was stirred at 25° C. for 48 hours and was concentrated under reduced pressure to remove EtOH. The residue was diluted with water (10 mL) and extracted with dichloromethane (15 mL*2). The combined organic phase was washed with brine (10 mL), dried over Na₂SO₄, filtered and concentrated under reduced pressure. The residue was purified by column chromatography (SiO₂, ethyl acetate:methanol=1:0 to 10:1) to give 5a. MS mass calculated for [M+H]⁺ (C₁₂H₁₄N₂O₂) requires m/z, 219.1, LCMS found m/z 219.1; ¹H NMR (400 MHz, CHLOROFORM-d) δ=9.28-9.11 (m, 1H), 8.40 (dd, J=2.0, 8.3 Hz, 1H), 7.63 (s, 1H), 7.58-7.48 (m, 1H), 5.43 (s, 1H), 3.54 (s, 6H), 2.76 (s, 3H)

Synthesis of 7-(dimethoxymethyl)-5-methyl-1,2,3,4-tetrahydro-1,8-naphthyridine (5b). To a solution of 2-(dimethoxymethyl)-4-methyl-1,8-naphthyridine (5a) (1.2 g, 5.50 mmol) in EtOH (20 mL) was added PtO₂ (12.49 mg, 54.98 umol) under N₂. The suspension was degassed under vacuum and purged with H₂ several times, then stirred under H₂ balloon (15 psi) at 25° C. for 18 hours. The reaction mixture was filtered through a Celite pad, the filter cake was rinsed with EtOH (10 mL*3). The combined filtrate was concentrated under reduced pressure to give 5b. MS mass calculated for [M+H]⁺ (C₁₂H₁₈N₂O₂) requires m/z, 223.1, LCMS found m/z 223.1; ¹H NMR (400 MHz, CHLOROFORM-d) δ=6.62 (s, 1H), 5.12 (s, 1H), 4.89 (br s, 1H), 3.63-3.50 (m, 2H), 3.49-3.32 (m, 6H), 2.64 (t, J=6.6 Hz, 2H), 2.16 (s, 3H), 1.99-1.88 (m, 2H)

Synthesis of Phenyl 7-(dimethoxymethyl)-5-methyl-3,4-dihydro-1,8-naphthyridine-1(211)-carboxylate (5c). To a mixture of 7-(dimethoxymethyl)-5-methyl-1,2,3,4-tetrahydro-1,8-naphthyridine (5b) (300 mg, 1.35 mmol) and diphenyl carbonate (289.11 mg, 1.35 mmol) in THF (5 mL) was added LiHMDS (1 M, 1.75 mL) dropwise at −25° C. under N₂. The mixture was stirred at −25° C. for 4 hrs and was warmed to 0° C. and quenched with saturated NH₄Cl aqueous solution (5 mL). The mixture was extracted with ethyl acetate (10 mL). The organic phase was washed with brine (10 mL), dried over Na₂SO₄, filtered, and concentrated under reduced pressure. The residue was purified by prep-HPLC (neutral condition; column: Waters Xbridge Prep OBD C18 150*40 mm*10 um; mobile phase: [water (10 mM NH₄HCO₃)-ACN]; B %: 25%-55%, 8 min) to give 5c. MS mass calculated for [M+H]⁺ (C₁₉H₂₂N₂O₄) requires m/z, 343.2, LCMS found m/z 343.2; ¹H NMR (400 MHz, CHLOROFORM-d) δ=7.40-7.33 (m, 2H), 7.27 (s, 1H), 7.24 (s, 1H), 7.22 (s, 1H), 7.20 (s, 1H), 5.25 (s, 1H), 4.01-3.87 (m, 2H), 3.39 (s, 6H), 2.77 (t, J=6.6 Hz, 2H), 2.30 (s, 3H), 2.16-2.02 (m, 2H)

Synthesis of N-(5-((2,6-difluoro-3,5-dimethoxybenzyl)oxy)pyrimidin-2-yl)-7-(dimethoxymethyl)-5-methyl-3,4-dihydro-1,8-naphthyridine-1(2H)-carboxamide (5d). To a solution of 5-((2,6-difluoro-3,5-dimethoxybenzyl)oxy)pyrimidin-2-amine (1h) (250 mg, 841.02 umol) in THF (2 mL) was added LiHMDS (1 M, 1.68 mL) at 0° C. Then phenyl 7-(dimethoxymethyl)-5-methyl-3,4-dihydro-1,8-naphthyridine-1(2H)-carboxylate (5c) (299.47 mg, 874.66 umol) in THF (4 mL) was added to the mixture at 0° C. and stirred for 2 hrs. The reaction mixture was cooled to 0° C. and quenched with saturated NH₄Cl aqueous solution (10 mL) and extracted with ethyl acetate (20 mL*2). The combined organic phase was washed with brine (10 mL), dried over Na₂SO₄, filtered, and concentrated under reduced pressure. The residue was purified by Prep-HPLC (column: Welch Xtimate C18 250*50 mm*10 um; mobile phase: [water (10 mM NH₄HCO₃)-ACN]; B %: 35%-75%, 10min) to give 5d. MS mass calculated for [M+H]⁺ (C₂₆H₂₉F₂N₅O₆) requires m/z, 546.2, LCMS found m/z 546.2; ¹H NMR (400 MHz, CHLOROFORM-d) δ=13.75 (s, 1H), 8.40 (s, 2H), 7.08 (s, 1H), 6.68 (t, J=8.1 Hz, 1H), 5.39 (s, 1H), 5.21 (s, 2H), 4.17-4.02 (m, 2H), 3.89 (s, 6H), 3.46 (s, 6H), 2.73 (t, J=6.4 Hz, 2H), 2.30 (s, 3H), 2.05-1.96 (m, 2H)

Synthesis of N-(5-((2,6-difluoro-3,5-dimethoxybenzyl)oxy)pyrimidin-2-yl)-7-formyl-5-methyl-3,4-dihydro-1,8-naphthyridine-1(2H)-carboxamide (Example 5). A solution of N-(5-((2,6-difluoro-3,5-dimethoxybenzyl)oxy)pyrimidin-2-yl)-7-(dimethoxymethyl)-5-methyl-3,4-dihydro-1,8-naphthyridine-1(2H)-carboxamide (5d) (20 mg, 36.66 umol) in CH₃CN (1 mL) was added aqueous HCl (0.2 mL, 1 M) and stirred for 2 hrs at 25° C. The reaction mixture was concentrated under reduced pressure to give Example 5. MS mass calculated for [M+H]⁺ (C₂₄H₂₃F₂N₅O₅) requires m/z, 500.2, LCMS found m/z 500.0; ¹H NMR (400 MHz, DMSO-d₆) δ=13.37 (s, 1H), 9.91 (s, 1H), 8.53 (s, 2H), 7.60 (s, 1H), 7.10 (br t, J=8.3 Hz, 1H), 5.28 (s, 2H), 3.98-3.94 (m, 2H), 3.89 (s, 6H), 2.84 (br t, J=6.4 Hz, 2H), 2.38 (s, 3H), 1.96 (br d, J=5.4 Hz, 2H)

### Example S5: Synthesis of N-(5-(2,6-difluoro-3,5-dimethoxyphenethyl)pyrimidin-2-yl)-7-formyl-3,4-dihydro-1,8-naphthyridine-1(2H)-carboxamide

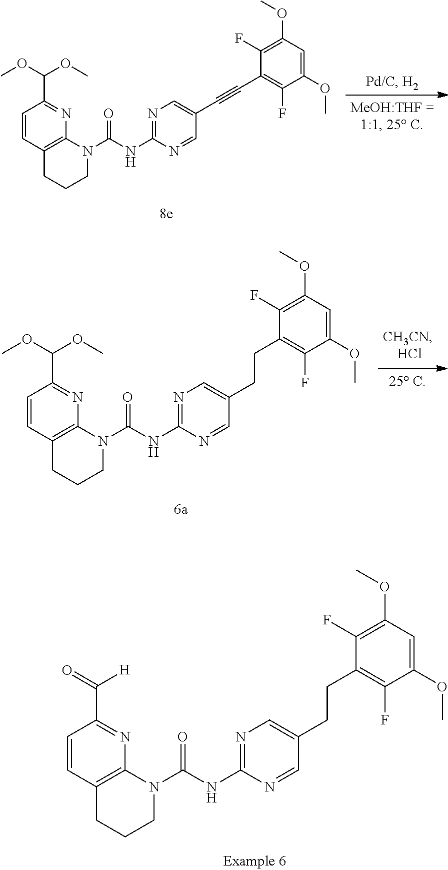

Synthesis of N-(5-(2,6-difluoro-3,5-dimethoxyphenethyl)pyrimidin-2-yl)-7-(dimethoxymethyl)-3,4-dihydro-1,8-naphthyridine-1(2H)-carboxamide (6a). To a solution of N-[5-[2-(2,6-difluoro-3,5-dimethoxy-phenyl)ethynyl]pyrimidin-2-yl]-7-(dimethoxymethyl)-3,4-dihydro-2H-1,8-naphthyridine-1-carboxamide (8e) (80 mg, 152.24 umol) in MeOH (2 mL) and THF (2 mL) was added Pd/C (30 mg, 152.24 umol, 10% purity) under H₂. The suspension was degassed under vacuum and purged with H₂ several times. The mixture was stirred under H₂ (40 psi) at 25° C. for 16 hrs and was filtered and the filter was concentrated to give 6a. MS mass calculated for [M+H]⁺ (C₂₆H₂₉F₂N₅O₅) requires m/z, 530.2, LCMS found m/z 530.3

Synthesis of N-(5-(2,6-difluoro-3,5-dimethoxyphenethyl)pyrimidin-2-yl)-7-formyl-3,4-dihydro-1,8-naphthyridine-1(2H)-carboxamide (Example 6). A mixture of N-[5-[2-(2,6-difluoro-3,5-dimethoxy-phenyl)ethyl]pyrimidin-2-yl]-7-(dimethoxymethyl)-3,4-dihydro-2H-1,8-naphthyridine-1-carboxamide (6a) (80 mg, 151.08 umol), aqueous HCl (1 M, 1.60 mL), CH₃CN (8 mL) was degassed and purged with N2 3 times and was stirred at 25° C. for 16 hrs under N₂ atmosphere. The reaction mixture was concentrated under reduced pressure to give a residue. The residue was purified by prep-HPLC (TFA condition: column: Nano-micro Kromasil C18 80*25 mm 3 um; mobile phase: [water (0.1% TFA)-ACN]; B %: 32%-52%, 7 min) to give Example 6. MS mass calculated for [M+H]⁺ (C₂₄H₂₃F₂N₅O₄) requires m/z, 484.2, LCMS found m/z 484.2; ¹H NMR (400 MHz, CHLOROFORM-d) δ=2.05 (br s, 2 H) 2.89 (br d, J=7.28 Hz, 2 H) 2.93-3.04 (m, 4 H) 3.85 (s, 6 H) 4.09-4.18 (m, 2 H) 6.52 (t, J=8.16 Hz, 1 H) 7.60-7.65 (m, 1 H) 7.67 (s, 1 H) 8.43 (s, 2 H) 10.11 (s, 1 H) 13.82 (br s, 1 H)

### Example S6: Synthesis of (E)-N-(5-(2,6-difluoro-3,5-dimethoxystyryl)pyrimidin-2-yl)-7-formyl-3,4-dihydro-1,8-naphthyridine-1(2H)-carboxamide

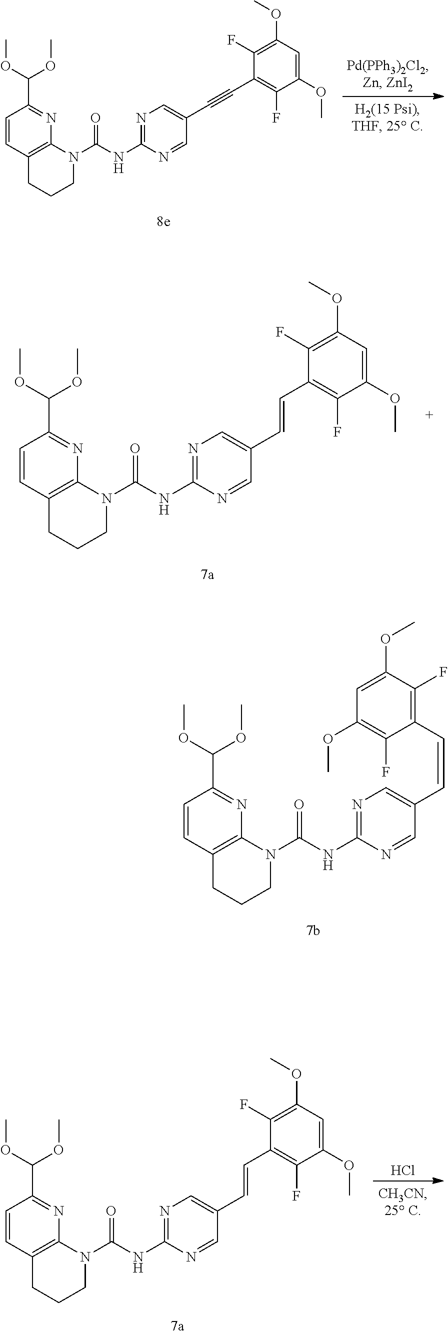

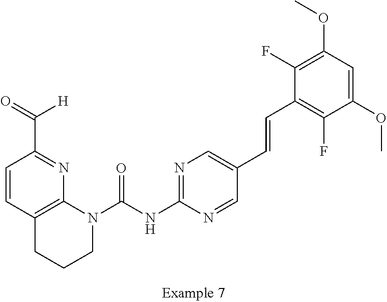 [SMILES: COc1cc(OC)c(F)c(/C=C/c2cnc(NC(=O)N3CCCc4ccc(C=O)nc43)nc2)c1F]

Synthesis of (E)-N-(5-(2,6-difluoro-3,5-dimethoxystyryl)pyrimidin-2-yl)-7-(dimethoxymethyl)-3,4-dihydro-1,8-naphthyridine-1(2H)-carboxamide (7a) & (Z)-N-(5-(2,6-difluoro-3,5-dimethoxystyryl)pyrimidin-2-yl)-7-(dimethoxymethyl)-3,4-dihydro-1,8-naphthyridine-1(2H)-carboxamide (7b). To a solution of N-[5-[2-(2,6-difluoro-3,5-dimethoxy-phenyl)ethynyl]pyrimidin-2-yl]-7-(dimethoxymethyl)-3,4-dihydro-2H-1,8-naphthyridine-1-carboxamide (8e) (100 mg, 190.29 umol) in THF (10 mL) was added diiodozinc (121.48 mg, 380.59 umol), Zn (37.33 mg, 570.88 umol), Pd(PPh₃)₂Cl₂ (26.71 mg, 38.06 umol) under N₂ at 25°. The mixture was degassed and purged with H₂ 3 times and was stirred at 25° C. for 16 hrs under H₂ (15 Psi). The reaction mixture was filtered and the filtrate was concentrated under reduced pressure to remove THF. The residue was purified by prep-HPLC (neutral condition: column: Waters Xbridge BEH C18 100*30 mm*10 um; mobile phase: [water (10 mM NH₄HCO₃)-ACN]; B %: 35%-65%, 10 min to afford 7a. MS mass calculated for [M+H]⁺ (C₂₆H₂₇F₂N₅O₅) requires m/z, 528.20, LCMS found m/z 528.2, ¹H NMR (400 MHz, CHLOROFORM-d) δ=8.71 (s, 2H), 7.46 (d, J=7.6 Hz, 1H), 7.31-7.22 (m, 1H), 7.11 (d, J=7.5 Hz, 1H), 7.08-7.01 (m, 1H), 6.52 (t, J=8.1 Hz, 1H), 5.38 (s, 1H), 4.11-4.00 (m, 2H), 3.83 (s, 6H), 3.42 (s, 6H), 2.84-2.73 (m, 2H), 1.98-1.81 (m, 2H); 7b. MS mass calculated for [M+H]⁺ (C₂₆H₂₇F₂N₅O₅) requires m/z, 528.2, LCMS found m/z 528.2, ¹H NMR (400 MHz, CHLOROFORM-d) δ=8.71 (s, 2H), 7.46 (d, J=7.6 Hz, 1H), 7.31-7.22 (m, 1H), 7.11 (d, J=7.5 Hz, 1H), 7.08-7.01 (m, 1H), 6.52 (t, J=8.1 Hz, 1H), 5.38 (s, 1H), 4.11-4.00 (m, 2H), 3.83 (s, 6H), 3.42 (s, 6H), 2.84-2.73 (m, 2H), 1.98-1.81 (m, 2H)

Synthesis of (E)-N-(5-(2,6-difluoro-3,5-dimethoxystyryl)pyrimidin-2-yl)-7-formyl-3,4-dihydro-1,8-naphthyridine-1(2H)-carboxamide (Example 7). To a solution of (E)-N-(5-(2,6-difluoro-3,5-dimethoxystyryl)pyrimidin-2-yl)-7-(dimethoxymethyl)-3,4-dihydro-1,8-naphthyridine-1(2H)-carboxamide (7a) (10 mg, 18.96 umol) in MeCN (2 mL) was added aqueous HCl (1 M, 94.78 uL), the mixture was degassed and purged with N₂ 3 times, and was stirred at 25° C. for 4 hrs under N₂ atmosphere. The reaction mixture was concentrated under reduced pressure. The residue was purified by prep-HPLC (TFA condition:column:Welch Ultimate AQ-C18 150*30 mm*5 um; mobile phase: [water (0.1% TFA)-ACN]; B %: 35%-65%, 10 min) to give Example 7. MS mass calculated for [M+H]⁺ (C₂₄H₂₁F₂N₅O₄) requires m/z, 482.2, LCMS found m/z 482.1; ¹H NMR (400 MHz, CHLOROFORM-d) δ=14.08 (br s, 1H), 10.13 (s, 1H), 8.83 (br s, 2H), 7.73-7.68 (m, 1H), 7.67-7.63 (m, 1H), 7.38-7.31 (m, 1H), 7.20-7.13 (m, 1H), 6.62 (t, J=7.9 Hz, 1H), 4.17 (br s, 2H), 3.91 (s, 6H), 2.97 (br t, J=6.2 Hz, 2H), 2.07-2.02 (m, 2H)

### Example S7: Synthesis of N-(5-((2,6-difluoro-3,5-dimethoxyphenyl)ethynyl)pyrimidin-2-yl)-7-formyl-3,4-dihydro-1,8-naphthyridine-1(2H)-carboxamide

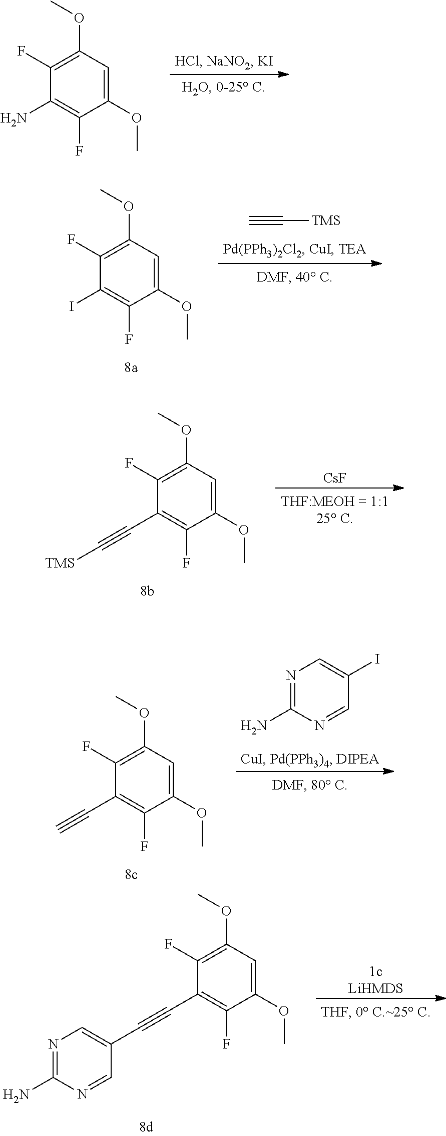

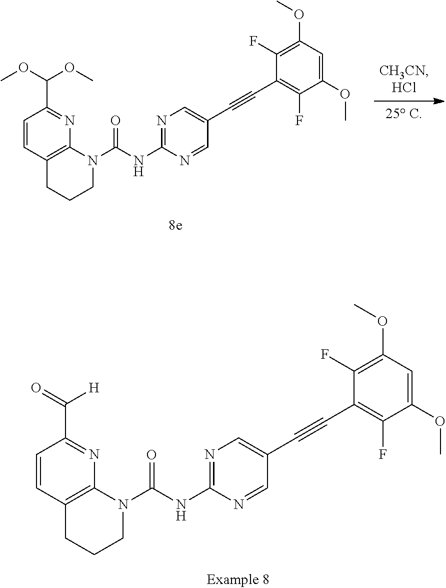

Synthesis of 2,4-difluoro-3-iodo-1,5-dimethoxybenzene (5a). To a solution of 2,6-difluoro-3,5-dimethoxy-aniline (3 g, 15.86 mmol) in aqueous HCl (6M, 27 mL) was added dropwise NaNO₂ (1.15 g, 16.65 mmol) in H₂O (6 mL) over 15 min at 0° C. After 15 min, the resulting slurry was added to a solution of KI (10.53 g, 63.44 mmol) in H₂O (15 mL) in small portions at 0° C. and the mixture was stirred at 25° C. for lhr. The reaction mixture was filtered and the solid was washed with H₂O (50 mL) and purified by column chromatography (SiO₂, petroleum ether:ethyl acetate=10:1 to 5:1) to give 8a. ¹H NMR (400 MHz, DMSO-d6) δ=3.89 (s, 6 H) 6.69 (t, J=7.94 Hz, 1H)

Synthesis of ((2,6-difluoro-3,5-dimethoxyphenyl)ethynyl)trimethylsilane (8b). A mixture of 2,4-difluoro-3-iodo-1,5-dimethoxy-benzene (8a) (3.3 g, 11.00 mmol), Et₃N (2.23 g, 22.00 mmol, 3.06 mL), CuI (418.93 mg, 2.20 mmol) and (trimethylsilyl)acetylene (21.61 g, 219.97 mmol, 30.47 mL), Pd(PPh₃)₂Cl₂ (385.99 mg, 549.93 umol) in DMF (20 mL) was degassed and purged with N₂ 3 times. The mixture was stirred at 40° C. for 16 hrs under N₂ atmosphere in a sealed tube. The reaction mixture was quenched by addition of H₂O (80 mL), and then extracted with ethyl acetate (50 mL*2). The combined organic layers were washed with brine (50 mL*2), dried over with Na₂SO₄, filtered and concentrated under reduced pressure to give a residue. The residue was purified by column chromatography (SiO₂, petroleum ether:ethyl acetate=10:1 to 5:1) to give 8b. ¹H NMR (400 MHz, CHLOROFORM-d) δ=0.29 (s, 9 H) 3.87 (s, 6 H) 6.62 (t, J=8.01 Hz, 1 H)

Synthesis of 3-ethynyl-2,4-difluoro-1,5-dimethoxybenzene (8c). A mixture of 2-(2,6-difluoro-3,5-dimethoxy-phenyl)ethynyl-trimethyl-silane (8b) (2.6 g, 9.62 mmol), CsF (1.46 g, 9.62 mmol, 354.59 uL) in MeOH (10 mL) and THF (10 mL) was degassed and purged with N2 3 times and the mixture was stirred at 25° C. for 1 hr under N₂ atmosphere. The reaction mixture was diluted with H₂O (100 mL) and extracted with ethyl acetate (100 mL*2). The combined organic layers were washed with brine (100 mL*2), dried over Na₂SO₄, filtered and concentrated under reduced pressure to give a residue. The residue was purified by prep-HPLC (column: Phenomenex luna C18 250*50 mm*10 um; mobile phase: [water (0.225% FA)-ACN]; B %: 40%-70%, 20 min) to give 8c. ¹H NMR (400 MHz, CHLOROFORM-d) δ=3.53 (s, 1 H) 3.89 (s, 6 H) 6.66 (br t, J=8.01 Hz, 1 H)

Synthesis of 5-((2,6-difluoro-3,5-dimethoxyphenyl)ethynyl)pyrimidin-2-amine (8d). A mixture of 3-ethynyl-2,4-difluoro-1,5-dimethoxy-benzene (8c) (500 mg, 2.52 mmol), 5-iodopyrimidin-2-amine (557.61 mg, 2.52 mmol), CuI (48.05 mg, 252.31 umol), Pd(PPh₃)₄ (291.56 mg, 252.31 umol) and DIPEA (326.10 mg, 2.52 mmol, 439.49 uL) in DMF (10 mL) was degassed and purged with N₂ 3 times and then the mixture was stirred at 80° C. for 2 hrs under N₂ atmosphere. The reaction mixture was quenched by addition of H₂O (20 mL), and then extracted with ethyl acetate (20 mL*5). The combined organic layers were washed with brine (20 mL*2), dried over with Na₂SO₄, filtered and concentrated under reduced pressure to give a residue. The residue was stirred in ethyl acetate (15 mL) at 25° C. for 20 min and filtered; filter cake was dried in vacuum to give 8d. MS mass calculated for [M+H]⁺ (C₁₄H₁₁F₂N₃O₂) requires m/z, 292.1, LCMS found m/z 292.1

Synthesis of N-(5-((2,6-difluoro-3,5-dimethoxyphenyl)ethynyl)pyrimidin-2-yl)-7-(dimethoxymethyl)-3,4-dihydro-1,8-naphthyridine-1(2H)-carboxamide (8e). To a solution of 5-((2,6-difluoro-3,5-dimethoxyphenyl)ethynyl)pyrimidin-2-amine (8d) (500 mg, 1.72 mmol) in THF (10 mL) was added dropwise LiHMDS (1 M, 5.15 mL) at 0° C. After addition, the mixture was stirred at this temperature for 20 min, and then phenyl 7-(dimethoxymethyl)-3,4-dihydro-2H-1,8-naphthyridine-1-carboxylate (1c)(1.13 g, 3.43 mmol) in THF (10 mL) was added dropwise at 0° C. The resulting mixture was warmed to 25° C. and stirred for 16 hrs. The reaction mixture was quenched by addition saturated NH₄Cl (50 mL), and then extracted with ethyl acetate (30 mL*2). The combined organic layers were washed with brine (50 mL*2), dried over with Na₂SO₄, filtered and concentrated under reduced pressure to give a residue. The residue was purified by column chromatography (SiO₂, petroleum ether:ethyl acetate=2:1 to 0:1) to give 8e. MS mass calculated for [M+H]⁺ (C₂₆H₂₅F₂N₅O₅) requires m/z, 526.2, LCMS found m/z 526.2; ¹H NMR (400 MHz, CHLOROFORM-d) δ=1.96-2.08 (m, 2 H) 2.87 (t, J=6.25 Hz, 2 H) 3.49 (s, 6 H) 3.91 (s, 6 H) 4.07-4.17 (m, 2 H) 5.44 (s, 1 H) 6.68 (t, J=8.00 Hz, 1 H) 7.20 (d, J=7.63 Hz, 1 H) 7.54 (d, J=7.63 Hz, 1 H) 8.80 (s, 2 H) 14.15 (s, 1 H)

Synthesis of N-(5-((2,6-difluoro-3,5-dimethoxyphenyl)ethynyl)pyrimidin-2-yl)-7-formyl-3,4-dihydro-1,8-naphthyridine-1(211)-carboxamide (Example 8). To a solution of N-[5-[2-(2,6-difluoro-3,5-dimethoxy-phenyl)ethynyl]pyrimidin-2-yl]-7-(dimethoxymethyl)-3,4-dihydro-2H-1,8-naphthyridine-1-carboxamide (8e) (50 mg, 95.15 umol) in CH₃CN (5 mL) was added aqueous HCl (1 M, 1 mL). The mixture was stirred at 25° C. for 16 hrs. The reaction mixture was concentrated under reduced pressure to give a residue. The residue was purified by prep-HPLC (TFA condition:column:Welch Ultimate AQ-C18 150*30 mm*5 um; mobile phase: [water (0.1% TFA)-ACN]; B %: 53%-83%, 12 min) to give Example 8. MS mass calculated for [M+H]⁺ (C₂₄H₁₉F₂N₅O₄) requires m/z, 480.1, LCMS found m/z 480.2; ¹H NMR (400 MHz, CHLOROFORM-d) δ=2.04-2.12 (m, 2 H) 2.97 (t, J=6.28 Hz, 2 H) 3.92 (s, 6 H) 4.15-4.21 (m, 2 H) 6.69 (t, J=8.05 Hz, 1 H) 7.62-7.66 (m, 1 H) 7.68-7.72 (m, 1 H) 8.81 (s, 2 H) 10.13 (s, 1 H) 14.08 (s, 1 H)

### Biological Examples

### Example B1. FGFR Inhibition Assays

The potency of test compounds in inhibiting FGFR1 and FGFR4 was determined. 20 μM of [KKKSPGEYVNIEFG] peptide substrate was added to the reaction buffer (20 mM 4-(2-hydroxyethyl)-1-piperazineethanesulfonic acid (HEPES) (pH 7.5), 10 mM MgCl₂, 1 mM egtazic acid (EGTA), 0.02% BRIJ®-35, 0.02 mg/ml bovine serum albumin (BSA), 0.1 mM Na₃VO₄, 2 mM dithiothreitol (DTT), and 1% DMSO) for FGFR1. 0.2 mg/mL of poly[Glu:Tyr] (4:1) peptide substrate was added to the reaction buffer (20 mM 4-(2-hydroxyethyl)-1-piperazineethanesulfonic acid (HEPES) (pH 7.5), 10 mM MgC12, 1 mM egtazic acid (EGTA), 0.02% BRIJ®-35, 0.02 mg/ml bovine serum albumin (BSA), 0.1 mM Na₃VO₄, 2 mM dithiothreitol (DTT), and 1% DMSO) for FGFR4. Next, FGFR1 or FGFR4 was added to the substrate solution and gently mixed.

Test compounds were dissolved in 100% dimethyl sulphoxide (DMSO) and then three-fold serially diluted in DMSO using an epMotion® 5070 robotic pipettor. The diluted test compounds, from 0.00001 M to 5.08053E-10 M, were added to corresponding substrate solutions, and the resulting substrate/compound solutions were incubated for 20 min at room temperature. Radioactive ATP (³³P-ATP) was added to each substrate/compound solution to initiate the kinase activity of FGFR1 or FGFR4 and were incubated for 2 hours at room temperature (10 μM final [ATP]). Reactions were spotted onto P81 ion exchange paper, washed to remove excess radiolabeled ATP and kinase activity determined by quantification of radioactive spots.

The half maximal inhibitory concentration (IC₅₀) values for FGFR1 and FGFR4 were determined for each compound assayed and are shown in Table 2. Example 1 and Example 2 inhibit FGFR4 at a dramatically lower concentration than the same compounds inhibit FGFR1.

TABLE 2

| | FGFR1 IC₅₀ | FGFR4 IC₅₀ |
|---|---|---|
| Example | (μM) | (μM) |
| 1 | 5.6 | 0.0008 |
| 2 | >10 | 0.0059 |
| 3 | 0.14 | <0.0005 |
| 5 | 0.13 | 0.0019 |
| 6 | 0.063 | 0.0010 |
| 7 | 1.0 | 0.0009 |
| 8 | 0.33 | 0.0025 |

All publications, including patents, patent applications, and scientific articles, mentioned in this specification are herein incorporated by reference in their entirety for all purposes to the same extent as if each individual publication, including patent, patent application, or scientific article, were specifically and individually indicated to be incorporated by reference.

Although the foregoing invention has been described in some detail by way of illustration and example for purposes of clarity of understanding, it is apparent to those skilled in the art that certain minor changes and modifications will be practiced in light of the above teaching. Therefore, the description and examples should not be construed as limiting the scope of the invention.

1. A compound of Formula (I) 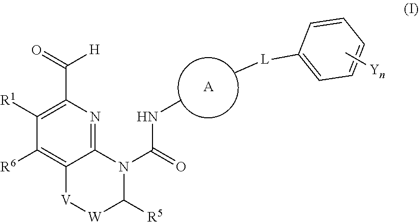
or a pharmaceutically acceptable salt thereof, wherein: 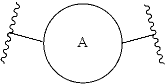
is 5- to 6-membered arylene or heteroarylene, each of which is optionally substituted by 1-4 halogen or C₁-C₆ alkyl groups;
L is —OCH₂—, —CH₂O—, —CH₂CH₂—, 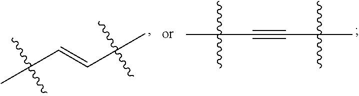
each Y is independently halogen or —O(C₁-C₆ alkyl) optionally substituted by 1-5 groups independently selected from the group consisting of halogen, hydroxyl, —CN, and —NH₂, provided that when 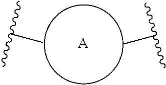
is 5-membered heteroarylene, then at least one Y, when present, is halogen;
n is 0-5;
V is CH₂, O, or CH(OH);
W is CH₂, CH₂CH₂, or a bond;
R¹ is H, halogen, hydroxyl, —CN, —NH₂, C₁-C₆ alkyl, C₁-C₆ haloalkyl, C₁-C₆ alkyl-OH, C₁-C₆ cycloalkyl, —CH₂NR²R³, —CH(CH₃)NR²R³, —O(C₁-C₆ alkyl), —CH₂CO₂H, —C(O)H, or 5- to 6-membered heterocyclyl or heteroaryl,
wherein the heterocyclyl or heteroaryl contains 1-3 heteroatoms selected from the group consisting of N, O, and S, and each of which heterocyclyl or heteroaryl is optionally substituted by 1-5 groups independently selected from the group consisting of C₁-C₆ alkyl, C₁-C₆ haloalkyl, halogen, hydroxyl, —CN, —NH₂, oxo, and 4- to 6-membered heterocyclyl containing 1-3 heteroatoms selected from the group consisting of N, O, and S;
R² is H, C₁-C₆ alkyl, C₁-C₆ haloalkyl, C₁-C₆ alkyl-OH, or (C₁-C₆ alkyl)₂N—(C₁-C₆ alkylene);
R³ is H, C₁-C₆ alkyl, —C(O)(C₁-C₆ alkyl), C₁-C₆ haloalkyl, C₁-C₆ alkyl-OH, —C(O)CH₂OH, —C(O)CH₂O(C₁-C₆ alkyl), —C(O)CH₂N(C₁-C₆ alkyl)₂, or —S(O)₂(C₁-C₆ alkyl);
or R² and R³ are taken together with the nitrogen atom to which they are attached to form a 5- to 6-membered heterocyclyl optionally containing one additional heteroatom or heteroatom-containing moiety selected from the group consisting of N, N-oxide, O, and S, wherein the heterocyclyl is optionally substituted by 1-5 R⁴ groups;
each R⁴ is independently:
halogen, —CN, C₁-C₆ alkyl, C₁-C₆ haloalkyl, C₁-C₆ alkyl-OH, —N(C₁-C₆ alkyl)₂, —C(O)(C₁-C₆ alkyl), or hydroxyl;
taken together with another R⁴ group and the carbon atom or atoms to which they are attached to form a spiro or fused 4- to 6-membered heterocyclyl containing 1-3 heteroatoms selected from the group consisting of N, O, and S; or
taken together with another R⁴ group attached to the same ring atom to form an oxo group;
R⁵ is H, C₁-C₆ alkyl, or C₁-C₆ cycloalkyl; and
R⁶ is H, halogen, C₁-C₆ alkyl, C₁-C₆ haloalkyl, or C₁-C₆ alkyl-OH.

2. The compound of claim 1, or a pharmaceutically acceptable salt thereof, wherein: 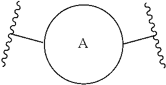
is phenylene or 5- to 6-membered heteroarylene containing 1-3 nitrogen atoms, each of which phenylene or heteroarylene is optionally substituted by 1-2 halogen or C₁-C₃ alkyl groups.

3. The compound of claim 2, or a pharmaceutically acceptable salt thereof, wherein: 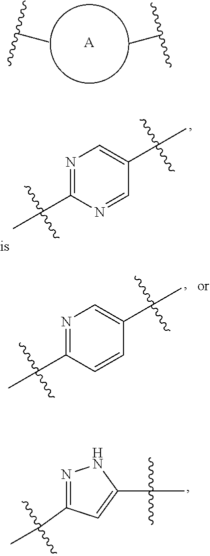
each of which is optionally substituted by 1-2 Cl or —CH₃ groups.

4. The compound of any one of claims 1-3, or a pharmaceutically acceptable salt thereof, wherein:
L is —OCH₂—.

5. The compound of any one of claims 1-3, or a pharmaceutically acceptable salt thereof, wherein:
L is —CH₂O—.

6. The compound of any one of claims 1-3, or a pharmaceutically acceptable salt thereof, wherein:
L is —CH₂CH₂—.

7. The compound of any one of claims 1-3, or a pharmaceutically acceptable salt thereof, wherein:
L is

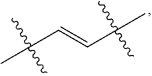 [SMILES: */C=C/*]

8. The compound of any one of claims 1-3, or a pharmaceutically acceptable salt thereof, wherein:
L is

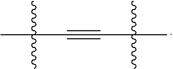 [SMILES: *C#C*]

9. The compound of any one of claims 1-8, or a pharmaceutically acceptable salt thereof, wherein:
each Y, where present, is independently halogen or —O(C₁-C₃ alkyl) optionally substituted by 1-3 groups independently selected from the group consisting of halogen, hydroxyl, —CN, and —NH₂.

10. The compound of claim 9, or a pharmaceutically acceptable salt thereof, wherein:
each Y is independently F, Cl, or —O(C₁-C₂ alkyl) optionally substituted by 1-2 groups independently selected from the group consisting of Cl, hydroxyl, —CN, and —NH₂.

11. The compound of claim 10, or a pharmaceutically acceptable salt thereof, wherein:
each Y is independently F, Cl, or —OCH₃.

12. The compound of any one of claims 1-11, or a pharmaceutically acceptable salt thereof, wherein:
n is 1-5.

13. The compound of claim 12, or a pharmaceutically acceptable salt thereof, wherein:
n is 4.

14. The compound of any one of claims 1-13, or a pharmaceutically acceptable salt thereof, wherein:
V is CH₂.

15. The compound of any one of claims 1-13, or a pharmaceutically acceptable salt thereof, wherein:
V is O or CH(OH).

16. The compound of any one of claims 1-15, or a pharmaceutically acceptable salt thereof, wherein:
W is CH₂.

17. The compound of any one of claims 1-15, or a pharmaceutically acceptable salt thereof, wherein:
W is CH₂CH₂ or a bond.

18. The compound of any one of claims 1-17, or a pharmaceutically acceptable salt thereof, wherein:
R¹ is H, halogen, hydroxyl, —CN, —NH₂, C₁-C₃ alkyl, C₁-C₃ haloalkyl, C₁-C₃ alkyl-OH, C₁-C₆ cycloalkyl, —CH₂NR²R³, —CH(CH₃)NR²R³, —O(C₁-C₃ alkyl), —CH₂CO₂H, —C(O)H, or 5- to 6-membered heterocyclyl or heteroaryl,
wherein the heterocyclyl or heteroaryl contains 1-3 heteroatoms selected from the group consisting of N, O, and S, and each of which heterocyclyl or heteroaryl is optionally substituted by 1-3 groups independently selected from the group consisting of C₁-C₃ alkyl, C₁-C₃ haloalkyl, halogen, hydroxyl, —CN, —NH₂, oxo, and 4- to 5-membered heterocyclyl containing 1-2 heteroatoms selected from the group consisting of N and O.

19. The compound of claim 18, or a pharmaceutically acceptable salt thereof, wherein:
R¹ is H, Cl, —CH₃, hydroxyl, —CN, —NH₂, —CF₃, —CH₂OH, cyclohexyl, —CH₂NR²R³, —CH(CH₃)NR²R³, —OCH₃, —CH₂CO₂H, —C(O)H, or 5- to 6-membered heterocyclyl or heteroaryl,
wherein the heterocyclyl or heteroaryl contains 1-2 heteroatoms selected from the group consisting of N, O, and S, and each of which heterocyclyl or heteroaryl is optionally substituted by 1-3 groups independently selected from the group consisting of —CH₃, —CF₃, Cl, F, hydroxyl, —CN, —NH₂, oxetanyl, and oxo.

20. The compound of claim 19, or a pharmaceutically acceptable salt thereof, wherein:
R¹ is H.

21. The compound of claim 19, or a pharmaceutically acceptable salt thereof, wherein:
R¹ is —CH₂NR²R³.

22. The compound of claim 19 or 21, or a pharmaceutically acceptable salt thereof, wherein:
R² is H, C₁-C₃ alkyl, C₁-C₃ haloalkyl, C₁-C₃ alkyl-OH, or (C₁-C₃ alkyl)₂N—(C₁-C₃ alkylene); and
R³ is H, C₁-C₃ alkyl, C₁-C₃ haloalkyl, C₁-C₃ alkyl-OH, —C(O)(C₁-C₃ alkyl), —C(O)CH₂OH, —C(O)CH₂O(C₁-C₃ alkyl), —C(O)CH₂N(C₁-C₃ alkyl)₂, or —S(O)₂(C₁-C₃ alkyl);
or R² and R³ are taken together with the nitrogen atom to which they are attached to form a 5- to 6-membered heterocyclyl optionally containing one additional heteroatom or heteroatom-containing moiety selected from the group consisting of N, N-oxide, O, and S, wherein the heterocyclyl is optionally substituted by 1-5 R⁴ groups.

23. The compound of claim 22, or a pharmaceutically acceptable salt thereof, wherein:
R² is H, —CH₃, —CF₃, —CH₂OH, or (CH₃)₂N—CH₂—; and
R³ is H, —CH₃, —CF₃, —CH₂OH, —C(O)(CH₃), —C(O)CH₂OH, —C(O)CH₂OCH₃, —C(O)CH₂N(CH₃)₂, or —S(O)₂CH₃;
or R² and R³ are taken together with the nitrogen atom to which they are attached to form a 6-membered heterocyclyl optionally containing one additional heteroatom selected from the group consisting of N and O, wherein the heterocyclyl is optionally substituted by 1-5 R⁴ groups.

24. The compound of any one of claims 21-23, or a pharmaceutically acceptable salt thereof, wherein:
R² and R³ are taken together with the nitrogen atom to which they are attached to form 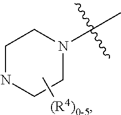
wherein the nitrogen atom at the 4-position is bound to H when not substituted by R⁴.

25. The compound of any one of claims 22-24, or a pharmaceutically acceptable salt thereof, wherein:
each R⁴, where present, is independently:
halogen, —CN, C₁-C₃ alkyl, C₁-C₃ haloalkyl, C₁-C₃ alkyl-OH, —N(C₁-C₃alkyl)₂, —C(O)(C₁-C₃ alkyl), or hydroxyl;
taken together with another R⁴ group and the carbon atom or atoms to which they are attached to form a spiro or fused 4- to 6-membered heterocyclyl containing 1-2 heteroatoms selected from the group consisting of N, O, and S; or
taken together with another R⁴ group attached to the same ring atom to form an oxo group.

26. The compound of claim 25, or a pharmaceutically acceptable salt thereof, wherein:
each R⁴ is independently:
Cl, F, —CN, —CH₃, —N(CH₃)₂, —C(O)CH₃, or hydroxyl;
taken together with another R⁴ group and the carbon atom or atoms to which they are attached to form a spiro or fused 4- to 6-membered heterocyclyl containing 1-2 heteroatoms selected from the group consisting of N, O, and S; or
taken together with another R⁴ group attached to the same ring atom to form an oxo group.

27. The compound of claim 26, or a pharmaceutically acceptable salt thereof, wherein:
each R⁴ is —CH₃.

28. The compound of claim 26, or a pharmaceutically acceptable salt thereof, wherein:
two R⁴ groups attached to the same ring atom are taken together to form an oxo group.

29. The compound of any one of claims 1-28, or a pharmaceutically acceptable salt thereof, wherein:
R⁵ is H, C₁-C₃ alkyl, or C₃-C₅ cycloalkyl.

30. The compound of claim 29, or a pharmaceutically acceptable salt thereof, wherein:
R⁵ is H, —CH₃, or cyclopropyl.

31. The compound of claim 30, or a pharmaceutically acceptable salt thereof, wherein:
R⁵ is H.

32. The compound of any one of claims 1-31, or a pharmaceutically acceptable salt thereof, wherein:
R⁶ is H, halogen, C₁-C₃ alkyl, C₁-C₃ haloalkyl, or C₁-C₃ alkyl-OH.

33. The compound of claim 32, or a pharmaceutically acceptable salt thereof, wherein:
R⁶ is H, Cl, —CH₃, —CF₃, or —CH₂OH.

34. The compound of claim 33, or a pharmaceutically acceptable salt thereof, wherein:
R⁶ is H or —CH₃.

35. A compound selected from the compounds in Table 1, or a pharmaceutically acceptable salt thereof.

36. A pharmaceutical composition comprising the compound of any one of claims 1-35, or a pharmaceutically acceptable salt thereof, and at least one pharmaceutically acceptable carrier, diluent, or excipient.

37. A method of treating cancer in a patient in need thereof, comprising administering to the patient a therapeutically effective amount of the compound of any one of claims 1-35, or a pharmaceutically acceptable salt thereof, or a therapeutically effective amount of the pharmaceutical composition of claim 36.

38. The method of claim 37, wherein the cancer is liver, colorectal, anal, breast, gastrointestinal, skin, stomach, esophageal, or pancreatic cancer.

39. The method of claim 38, wherein the cancer originates from the liver or spreads to the liver.

40. The method of any one of claims 37-39, wherein the cancer is hepatocellular carcinoma (HCC).

41. The method of any one of claims 37-40, further comprising administering one or more additional pharmaceutical agents.

42. The method of claim 41, wherein the one or more additional pharmaceutical agents is selected from the group consisting of cabozantinib-S-malate, pembrolizumab, lenvatinib mesylate, sorafenib tosylate, nivolumab, ramucirumab, and regorafenib.
